(19) United States
(12) Patent Application Publication
(10) Pub. No.: US 2009/0291971 A1
(43) Pub. Date: Nov. 26, 2009

(54) HETEROARYL ETHERS AND PROCESSES FOR THEIR PREPARATION

(75) Inventors: Tarek Suhayl Mansour, New City, NY (US); Sumrit Wacharasindhu, Bangkok (TH); Zhao-Kui Wan, Lexington, MA (US); Sujata Bardhan, New York, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/261,469

(22) Filed: Oct. 30, 2008

Related U.S. Application Data
(60) Provisional application No. 60/984,477, filed on Nov. 1, 2007.

(51) Int. Cl. A61K 31/519; C07D 239/72; A61K 31/517; A61K 31/505; A61P 29/00; A61P 35/00; C07D 239/24; C07D 495/02

(52) U.S. Cl. 514/260.1; 544/284; 514/266.23; 544/278; 544/316; 514/274; 544/298; 514/269; 514/266.21; 514/266.2; 514/266.24

(57) ABSTRACT

The present invention relates to processes for the preparation of heteroaryl ethers. In some embodiments, the processes relate to cross coupling reactions between triazol-1-yloxy and triazol-1-yl heterocycles with aryl boronic acids. In a further aspect, this invention also relates to compounds that are useful for the treatment of oncological diseases or disorders, and for the treatment of inflammation.

### CROSS REFERENCE TO RELATED APPLICATIONS

This application claims priority benefit of U.S. Provisional Application Ser. No. 60/984,477, filed Nov. 1, 2007, the entire content of which is incorporated herein by reference.

### FIELD OF THE INVENTION

In one aspect, this invention relates to processes for the preparation of heteroaryl ethers. In some embodiments, the processes relate to cross coupling reactions between triazol-1-yloxy and triazol-1-yl heterocycles with aryl boronic acids. In a further aspect, this invention also relates to compounds that are useful for the treatment of oncological diseases or disorders, and for the treatment of inflammation.

### BACKGROUND OF THE INVENTION

Transition metal-catalyzed cross coupling reactions have become important methods for nucleophilic aromatic substitution reactions. These reactions involve the use of various metals such as copper, nickel, or palladium catalysts with an aromatic substrates carrying a leaving group such as halogen, triflate, tosylate, thioether paired with organostannanes, organoboronic acids or silanols to form C—C, C—N, C—O and C—S bonds. These reactions are commonly referred to as Stille, Suzuki-Miyaura Hiyama, Sonagashira, Kumada, Buchwald-Hartwig cross coupling reactions depending on the substrates.

The Pd-catalyzed formation of C—O bonds (O-Arylation) was first reported in 2001 by the reaction of primary alcohols with aryl chlorides and bromides to give aryl ethers. Tertiary alcohols, phenols and silanols undergo this reaction to form aryl ethers in an intermolecular fashion. To overcome problems of aryl homo coupling observed mainly with primary and secondary alcohols, bulky biaryl phosphine ligands on the palladium catalyst have been used. Heteroaromatics such as pyrimidines and hydroxypyridines and hydroxyquinolines undergo a Cu-mediated O-arylation with aryl bismuth reagents or aryl halides in low chemoselectivity.

Intramolecular O-arylation reactions of phenols and phenylboronic acids mediated by copper acetate have also been reported. In all these intermolecular or intramolecular reactions, the oxygen moiety is derived from the reactant alcohols, phenols or silanols.

Cyclic amides react with amines in the presence of 1-H-benzotriazol-1-yloxy-tris(dimethylamino) phosphonium hexafluorophosphate (BOP) or other phosphonium salts such as PyBOP and PyAOP to form cyclic amidines and cyclic guanidines. These reactions involve the formation of phosphonium salts and/or the benzotriazol-1-yloxy adducts (HOBt adducts). The HOBt adducts can react with nucleophiles such as amines, thiols, and phenols to effect an overall nucleophilic substitution reaction (S_{N}Ar) resulting in the formation of heteroaryl amines, thioethers and ethers. Given the importance of these compounds as, for example, therapeutics and intermediates toward the production of therapeutics, it can be seen that improved synthetic methodologies for their preparation are of great benefit. The present invention is directed to these, as well as other, other important ends.

### SUMMARY OF THE INVENTION

In some embodiments, the invention provides synthetic processes comprising reacting a compound of Formula II:

Ht-(O)ₖ-L II

or a salt thereof, wherein:

k is 0 or 1;

L is a group having the Formula:

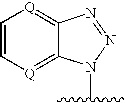

wherein one Q is CH, and one Q is CH or N;

Ht is a heterocycle of Formula a, b, c or d:

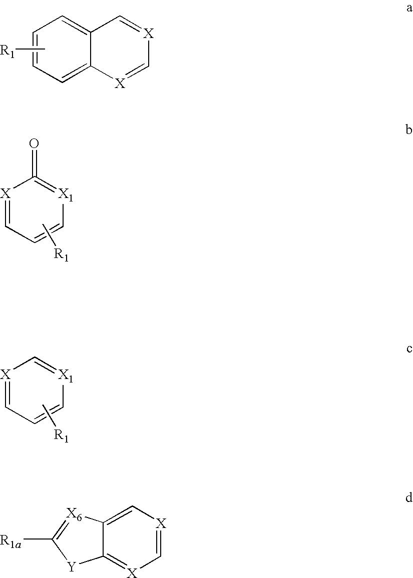

R₁, R₁ₐ and R_{1b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

each X is independently N or CH;

X₆ is CR_{1b} or N;

X₁ is NH or CH₂; and

Y is S or O;

with a compound of Formula Ar—B(OH)₂; wherein Ar has one of the Formulas e-j:

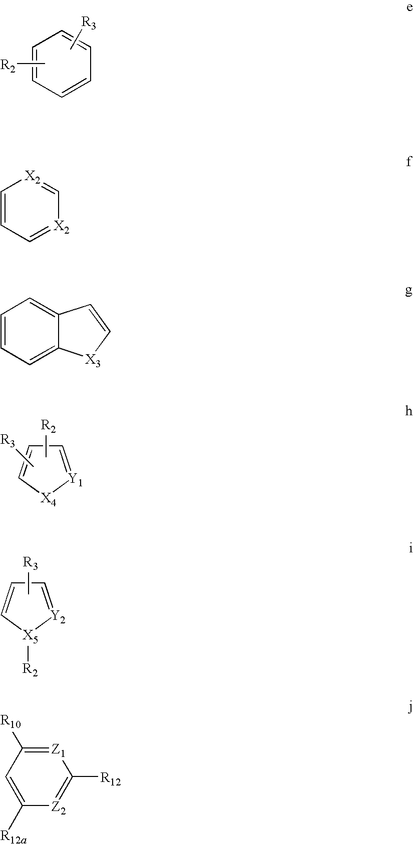

wherein:

each X₂ is independently N or CH;

X₃ is NH or CH₂;

X₄ is NH, S or O;

X₅ is N or CH;

Y₁ is N or CH;

Y₂ is N or CH;

R₂ and R₃ are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

Z₁ is CR₁₁ₐ or N;

Z₂ is CR_{11b} or N; and

R₁₀, R₁₁ₐ, R_{11b}, R₁₂ and R₁₂ₐ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;

wherein the reaction is performed in the presence of a base;

and optionally in the presence of water;

and optionally in the presence of one or more of:
(i) oxygen;
(ii) a Pd(0) catalyst; and
(iii) H₂O₂;
   for a time and under conditions effective to form a compound of Formula Ht-O—Ar.

In some embodiments, the base includes or consists of a metal carbonate or phosphate, for example a Group I or Group II metal carbonate, metal bicarbonate or phosphate, such as Cs₂CO₃, Na₂CO₃, NaHCO₃, K₂CO₃, KHCO₃ Na₃PO₄, K₃PO₄, K₂HPO₄ or Cs₃PO₄, with Cs₂CO₃, being preferred. In some embodiments, the reaction is performed in the presence of oxygen. In some embodiments, the reaction is performed in the presence of a catalyst, preferably a palladium (0) catalyst. In some preferred embodiments, the reaction is performed in the presence of oxygen and a palladium (0) catalyst. In some embodiments, the reaction is performed in the presence of H₂O₂.

In some embodiments, the palladium (0) catalyst includes one or more of bis[1,2-bis(diphenylphosphino)ethane]palladium(0), bis(dibenzylideneacetone) palladium(0), 1,3-Bis(2,6-diisopropylphenyl)imidazol-2-ylidene(1,4-naphthoquinone)palladium(0) dimer, bis(3,5,3′,5′-dimethoxydibenzylideneacetone) palladium(0), bis(tri-tert-butylphosphine)palladium(0), 1,3-bis(2,4,6-trimethylphenyl) imidazol-2-ylidene (1,4-naphthoquinone)palladium(0) dimer, tetrakis(methyldiphenyl phosphine)palladium(0), tetrakis(triphenylphosphine)palladium(0), tris(dibenzylidene acetone)dipalladium(0), tris(dibenzylideneacetone)dipalladium(0)-chloroform adduct, and tris(3,3′,3″-phosphinidynetris(benzenesulfonato)palladium(0) nonasodium salt nonahydrate, with tetrakis(triphenylphosphine)palladium(0); Pd(PPh₃)₄ being preferred. In some embodiments, the compound of Formula II is prepared by reacting a compound of Formula Ht-OH with a coupling reagent in the presence of a base, and under an inert atmosphere. In some such embodiments, the coupling reagent includes or consists of a phosphonium salt having a cation of Formula:

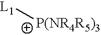

wherein:

L₁ is a moiety of Formula:

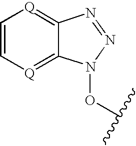

one Q is CH, and one Q is CH or N;

each R₄ and each R₅ is independently C₁₋₆ alkyl;

and wherein any R₄ and R₅ attached to the same nitrogen atom can together form a moiety of formula —(CH₂)_{q}— where q is 2, 3, 4, 5 or 6.

In some such embodiments, Q is CH, for example BOP. In some further such embodiments, the reacting of the compound of Formula Ht-OH with said coupling reagent is performed in the presence of benzotriazole.

In some further embodiments, Q is N, for example PyAOP. In some such embodiments, the reacting of the compound of Formula Ht-OH with said coupling reagent is performed in the presence of 3H-[1,2,3]triazolo[4,5-b]pyridine.

In some embodiments wherein k is 1, the compound of Formula II is prepared by reacting a compound of Formula Ht-OH with a coupling reagent in the presence of a base, and in the presence of oxygen, for example under an air atmosphere or one containing a greater amount of air, such as a pure oxygen atmosphere. In some such embodiments, the coupling reagent includes or consists of a phosphonium salt having a cation of Formula:

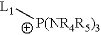

wherein:

L₁ is a moiety of Formula:

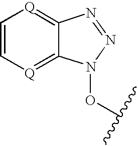

one Q is CH, and one Q is CH or N;

each R₄ and each R₅ is independently C₁₋₆ alkyl;

and wherein any R₄ and R₅ attached to the same nitrogen atom can together form a moiety of formula —(CH₂)_{q}— where q is 2, 3, 4, 5 or 6. In some such embodiments, Q is CH, for example BOP. In other such embodiments, Q is N, for example PyAOP.

In some further embodiments, the invention provides synthetic processes comprising:

(a) reacting a compound of Formula Ht-OH with a coupling reagent of Formula

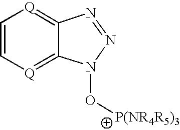

wherein:

one Q is CH, and one Q is CH or N;

each R₄ and R₅ is independently C₁₋₆ alkyl;

and wherein any R₄ and R₅ attached to the same nitrogen atom can together form a moiety of formula —(CH₂)_{q}— where q is 2, 3, 4, 5 or 6;

in the presence of a base, to form a compound of Formula II:

Ht-(O)ₖ-L II

or a salt thereof, wherein:

k is 0 or 1;

Ht is a heterocycle of Formula a, b, c or d:

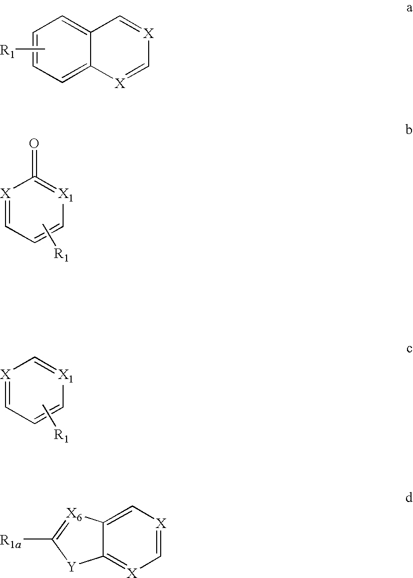

R₁, R₁ₐ and R_{1b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

each X is independently N or CH;

X₆ is CR_{1b} or N;

X₁ is NH or CH₂;

Y is S or O; and

L is a group having the Formula:

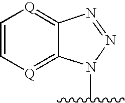

wherein one Q is CH and one Q is CH or N; and

(b) reacting said compound of Formula II with a compound of Formula Ar—B(OH)₂;

wherein Ar has one of the Formulas e-j:

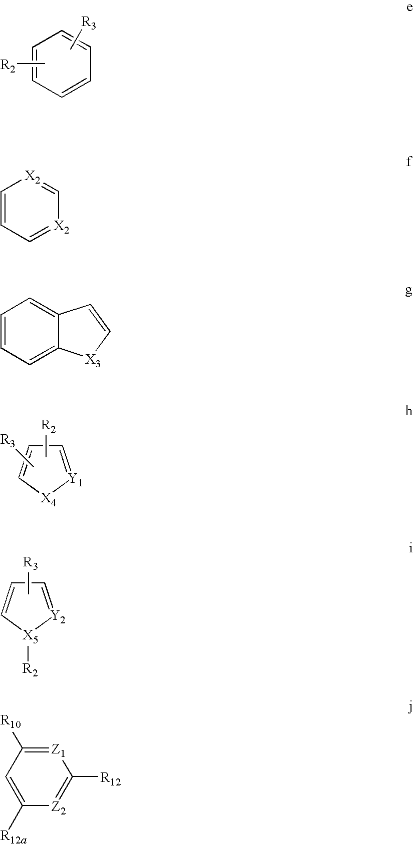

wherein:

each X₂ is independently N or CH;

X₃ is NH or CH₂;

X₄ is NH, S or O;

X₅ is N or CH;

Y₁ is N or CH;

Y₂ is N or CH;

R₂ and R₃ are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

Z₁ is CR₁₁ₐ or N;

Z₂ is CR_{11b} or N; and

R₁₀, R₁₁ₐ, R_{11b}, R₁₂ and R₁₂ₐ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;

in the presence of a metal carbonate base, a palladium (0) catalyst and oxygen, for a time and under conditions effective to form a compound of Formula Ht-O—Ar.

In some embodiments, the base is a Group I or Group II metal carbonate or bicarbonate or phosphate, such as Cs₂CO₃, Na₂CO₃, NaHCO₃, K₂CO₃, KHCO₃ Na₃PO₄, K₃PO₄, K₂HPO₄ or Cs₃PO₄, with Cs₂CO₃, being preferred.

In some embodiments, Ht has the Formula a, wherein each X is N. In some further embodiments, Ht has the Formula d, wherein each X is N and Y is S. In some further embodiments, Ht has the Formula c, wherein each X is N. In some further embodiments, Ht has the Formula b, wherein X is N, and X₁ is NH.

In some further embodiments, Ar has the Formula e. In some further embodiments, Ar has the Formula f, wherein one X₂ is N and the other X₂ is CH. In some further embodiments, Ar has the Formula f, wherein each X₂ is N. In some further embodiments, Ar has the Formula g, wherein X₃ is NH. In some further embodiments, Ar has the Formula h, wherein X₄ is 0, and Y₁ is N. In some further embodiments, Ar has the Formula i, and in some further embodiments, Ar has the Formula j.

In some preferred embodiments, Ht has the Formula a wherein each X is N, and Ar has the Formula j.

In a further aspect, the invention provides compounds of Formula III:

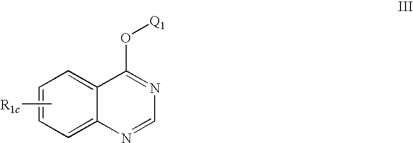

wherein:

R_{1c} is H, C₁₋₁₄ alkyl, C₁₋₁₄ alkoxy, halogen, C₇₋₂₀ arylalkyl, cyano, nitro, C₂₋₁₄ carboalkoxy, C₂₋₁₄ alkanoyl or trihaloalkyl;

Q₁ is selected from formulas j, k, m, n and o:

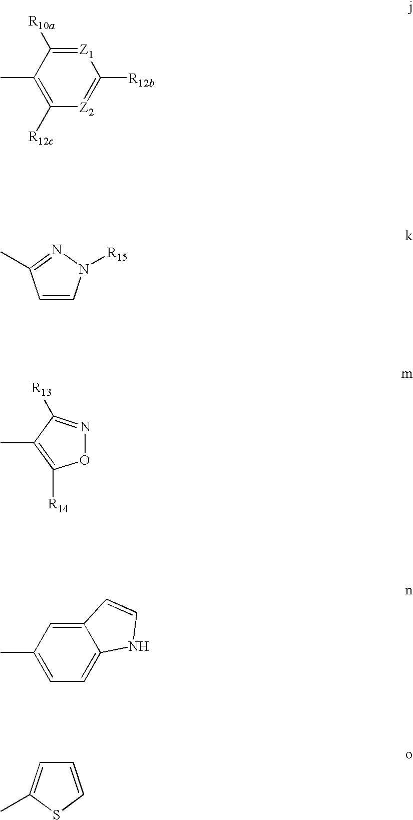

### Z₁ is CR₁₁ₐ or N;

### Z₂ is CR_{11b} or N;

R₁₀ₐ, R₁₁ₐ, R_{11b}, R_{12b} and R_{12c} are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;
R₁₃ and R₁₄ are each independently selected from the group consisting of H, C₁₋₁₄ alkyl and C₇₋₂₄ arylalkyl; and
R₁₅ is H, C₁₋₁₄ alkyl, C₇₋₂₄ arylalkyl, or C₁₋₆ trihaloalkyl;
provided that:

(i) when R₁₀ₐ and R_{12c} are each H, and Z₁ and Z₂ are each CH, then R_{12b} is not NO₂; and

(ii) when R_{12b} is H, and Z₁ and Z₂ are each CH, then neither R₁₀ₐ nor R_{12c} is NO₂.

In some embodiments, Q₁ has the Formula j, Z₁ is CR₁₁ₐ and Z₂ is CR_{11b}. In some such embodiments, Z₁ and Z₂ are each CH. In some further such embodiments, Z₁ and Z₂ are each CH and R_{12b} is H.

In some embodiments, Q₁ has the Formula j, Z₁ is CR₁₁ₐ, Z₂ is CR_{11b}, and R₁₀ₐ and R_{12b} are each H.

In some embodiments, Q₁ has the Formula j, Z₁ and Z₂ are each CH; and R₁₀ₐ and R_{12c} are each H.

In some embodiments, Q₁ has the Formula j, Z₁ is CR₁₁ₐ and Z₂ is CR_{11b}, and R_{12b} is selected from the group consisting of CF₃, —C(═O)CH₃, —C(═O)CH₂CH₃, —OCH₃, —OCH₂CH₃, —CH₃, —CH₂CH₃, CN, halogen and C₇₋₂₄ arylalkyl. In some such embodiments, Z₁ and Z₂ are each H.

In some embodiments, Q₁ has the Formula j, Z₁ and Z₂ are each CH, R₁₀ₐ and R_{12c} are each H, and R_{12b} is selected from the group consisting of CF₃, —C(═O)CH₃, —C(═O)CH₂CH₃, —OCH₃, —OCH₂CH₃, —CH₃, —CH₂CH₃, CN, halogen and C₇₋₂₄ arylalkyl.

In some embodiments, Q₁ has the Formula j, Z₁ is CR₁₁ₐ, and Z₂ is N. In some such embodiments, R_{12b} is halogen. In further such embodiments, R_{12c} is alkoxy.

In some embodiments, the invention provides compounds having the Formula IV:

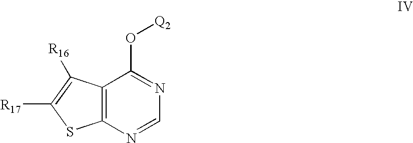

wherein:

R₁₆ and R₁₇ are each independently H, C₁₋₁₄ alkyl, carboxy, C₂₋₁₄ carboalkoxy, C(═O)CH₃, —C(═O)CH₂CH₃, C₂₋₁₄ alkanoyl or C₇₋₂₄ arylalkyl; and

Q₂ has the Formula:

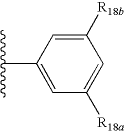

R₁₈ₐ and R_{18b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl, C₁₋₆ trihaloalkyl, N(R₅₀)(R₅₁), —NH₂, —NH(C₁-C₆ alkyl), —N(C₁-C₆ alkyl)(C₁-C₆ alkyl), —N(C₁-C₃ alkyl)C(O)(C₁-C₆ alkyl), —NHC(O)(C₁-C₆ alkyl), —NHC(O)H, —C(O)NH₂, —C(O)NH(C₁-C₆ alkyl), —C(O)N(C₁-C₆ alkyl)(C₁-C₆ alkyl), —CN, —OH, —C(O)OC₁-C₆ alkyl, —C(O)C₁-C₆ alkyl, C₆-C₁₄ aryl and C₄-C₁₀ heteroaryl;

R₅₀ and R₅₁ are each independently H, C₁₋₁₄ alkyl, C₂₋₁₄ alkenyl, C₂₋₁₄ alkynyl, C₇₋₂₄ arylalkyl, C₂₋₁₄ alkanoyl, C₁₋₆ trihaloalkyl, —S(O)ᵥ—C₁₋₁₄ alkyl; —S(O)ᵥ—C₆₋₁₄ aryl, —S(O)ᵥ—C₇₋₂₄ arylalkyl or —S(O)ᵥ—C₇₋₂₄ alkylaryl; where v is 0, 1 or 2;

or R₅₀ and R₅₁ together can form a moiety of formula —(CH₂)ᵣ— where r is 2, 3, 4, 5 or 6;

or Q₂ has the Formula m:

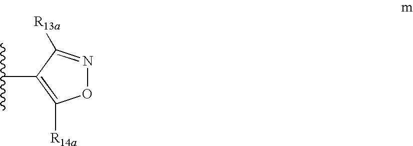

where R₁₃ₐ and R₁₄ₐ are each independently selected from the group consisting of H, C₁₋₁₄ alkyl and C₇₋₂₄ arylalkyl.

In some embodiments, Q₂ has the Formula:

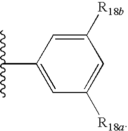

In some such embodiments, R₁₈ₐ and R_{18b} are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl.

In some embodiments, Q₂ has the Formula m:

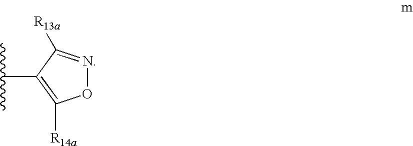

In some such embodiments, R₁₃ₐ and R₁₄ₐ are each C₁₋₁₄ alkyl.

In some embodiments, the invention further provides compounds of Formula V:

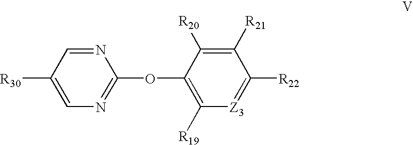

wherein:

### Z₃ is N or CR_{11c};

R_{11c} is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₁₉ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₀ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₁ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₂ is H F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl; and
R₃₀ is halogen;
provided that:

(i) when Z₃ is CR_{11c}, then at least one of R₁₉, R₂₀, R₂₁ and R₂₂ is other than H; and

(ii) when Z₃ is N, the R_{11c}, R₁₉, R₂₀, R₂₁ and R₂₂ are each independently selected from H, C₂₋₁₄ alkenyl, —S—C₁₋₁₄ alkyl and C₁₋₁₄ alkoxy.

In some embodiments, Z₃ is N. In some such embodiments, R₃₀ is bromine. In some such embodiments, R₁₉ is C₁₋₁₄ alkoxy. In some such embodiments, R₂₀, R₂₁ and R₂₂ are each H.

In some embodiments, Z₃ is CR_{11c}. In some such embodiments, R_{11c}, R₁₉, R₂₀, R₂₁ and R₂₂ are each H.

In some embodiments, the invention further provides compounds of Formula VI:

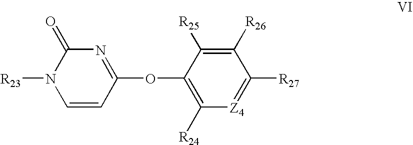

wherein:

### (A) Z₄ is N;

R₂₃ is C₁₋₁₄ alkyl or C₇₋₂₄ arylalkyl; and

R₂₄, R₂₅, R₂₆ and R₂₇ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl; or

### (B) Z₄ is CR_{11d};

R_{11d} is H or C₁₋₁₄ alkyl;

R₂₃ is C₁₋₁₄ alkyl; and

R₂₄, R₂₅, R₂₆ and R₂₇ are each independently selected from the group consisting of H, C₁₋₁₄ alkoxy, C₇₋₂₄ arylalkyl, cyano, C₂₋₁₄ carboalkoxy, C₂₋₁₄ alkanoyl and C₁₋₆ trihaloalkyl.

In some embodiments, Z₄ is N. In some such embodiments, R₂₄ is C₁₋₁₄ alkoxy. In further such embodiments, R₂₄, R₂₅, R₂₆ and R₂₇ are each independently selected from H, C₁₋₁₄ alkoxy, cyano and C₁₋₆ trihaloalkyl.

In some embodiments wherein Z₄ is N, R₂₄, R₂₅, R₂₆ and R₂₇ are each independently selected from H, OCH₃, cyano and CF₃.

In some embodiments, Z₄ is CR_{11d}. In some such embodiments, R₂₄ is C₁₋₁₄ alkoxy. In some such embodiments, R₂₃ is methyl; and R₂₄ is methoxy.

The invention further provides methods for treating an oncological disease or disorder, comprising administering to a patient in need thereof a therapeutically effective amount of a compound of the invention. The invention further provides methods for treating inflammation, comprising administering to a patient in need thereof a therapeutically effective amount of a compound of the invention.

### DESCRIPTION OF THE INVENTION

In one aspect, this invention relates to the novel reactions of benzotriazol-1-yloxy and pyridotriazol-1-yloxy adducts derived from mono and bicyclic heterocycles with aryl and heteroaryl boronic acids. In some embodiments, the reactions are performed in the presence of oxygen, or in the presence of hydrogen peroxide (H₂O₂). In some embodiments, the reactions are mediated by palladium (0) catalyst to afford heteroaryl ethers in excellent yields and high O versus N chemoselectivity. In the absence of oxygen, benzotriazol-yl or pyridotriazol-1-yl heterocyclic adducts are also formed. Thus, in a further aspect, this invention also relates to the palladium-catalyzed cross coupling reaction of benzotriaol-1-yl and pyridotriazol-1-yl heterocyclic adducts with aryl and heteroaryl boronic acids and oxygen to afford heteroaryl ethers in high O versus N chemoselectivity.

In accordance with some embodiments of the present invention, heteroaryl ethers can be prepared from the reaction of triazol-1-yloxy and triazol-1-yl heterocycles with aryl boronic acids in the presence of a base. In some embodiments, the reaction is performed in the presence of oxygen, or H₂O₂, or in the presence of a palladium(0) catalyst, or, in the presence of both oxygen and a palladium(0) catalyst. Accordingly, in some embodiments, the invention provides synthetic processes comprising reacting a compound of Formula II:

Ht-(O)ₖ-L II

or a salt thereof, wherein:

k is 0 or 1;

L is a group having the Formula:

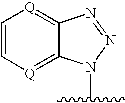

wherein one Q is CH, and one Q is CH or N;

Ht is a heterocycle of Formula a, b, c or d:

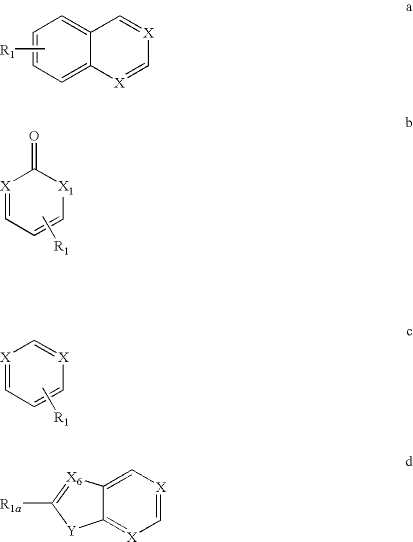

R₁, R₁ₐ and R_{1b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

each X is independently N or CH;

X₆ is CR_{11b} or N;

X₁ is NH or CH₂; and

Y is S or O;

with a compound of Formula Ar—B(OH)₂; wherein Ar has one of the Formulas e-j:

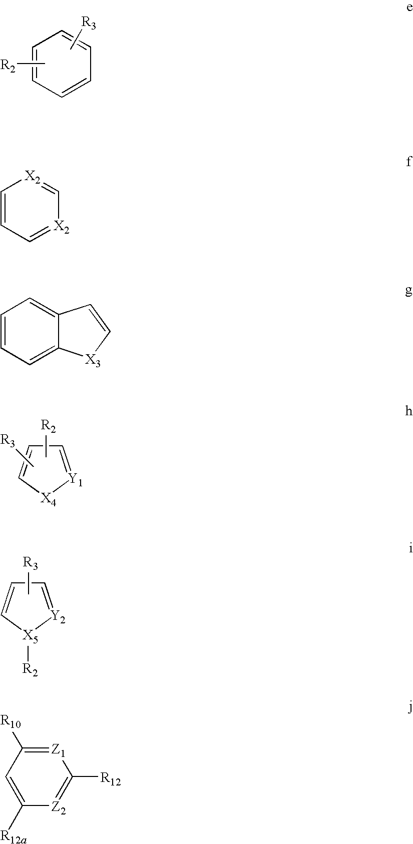

wherein:

each X₂ is independently N or CH;

X₃ is NH or CH₂;

X₄ is NH, S or O;

X₅ is N or CH;

Y₁ is N or CH;

Y₂ is N or CH;

R₂ and R₃ are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

Z₁ is CR₁₁ₐ or N;

Z₂ is CR_{11b} or N; and

R₁₀, R₁₁ₐ, R_{11b}, R₁₂ and R₁₂ₐ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;

wherein the reaction is performed in the presence of a base;

and optionally in the presence of water;

and optionally in the presence of one or more of:
(i) oxygen;
(ii) a Pd(0) catalyst; and
(iii) H₂O₂;
   for a time and under conditions effective to form a compound of Formula Ht-O—Ar.

The compound of Formula Ht-(O)ₖ-L can be prepared, for example, by reaction of the corresponding alcohol, Ht-OH, with a coupling reagent containing a triazolyl phosphonium ion. In accordance with certain embodiments of the invention, the coupling reagent can include a benzotriazolyl or pyridyltriazolyl phosphonium ion, as discussed further below. A summary of the reactions is shown in Schemes 1 and 2, below:

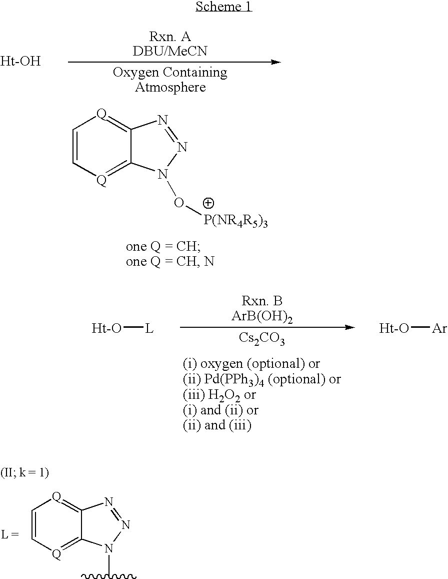

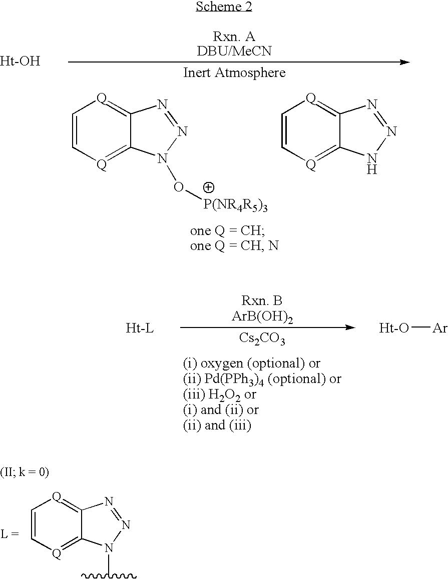

As can be seen in Scheme 1, in the presence of oxygen, the product of Reaction A is an intermediate triazolyl ether Ht-O-L (i.e., a compound of Formula II where k=1). In an inert atmosphere (Scheme 2), Reaction A produces both the intermediate triazolyl ether Ht-O-L, and a compound of Formula Ht-L (i.e., a compound of Formula II where k=0). In the context of the present invention, a reaction is performed “in the presence of oxygen” when it is performed under an atmosphere that contains oxygen or a source of oxygen capable of participating in the reaction to produce the indicated species. In embodiments where compounds of Formula II where k=1 are desired, the reaction is performed in the presence of oxygen, for example in the presence of air, or an atmosphere containing a higher percentage by weight of oxygen than air, up to and including a pure oxygen atmosphere. As used herein, the term “inert atmosphere” denotes an atmosphere that is devoid of oxygen, or that does not participate in the indicated reaction. Examples of inert atmospheres include noble gases such as helium, argon, neon and krypton, and nitrogen gas. Additional examples will be apparent to those of skill in the art.

As further shown in Reaction B of Schemes 1 and 2, either intermediate compound Ht-O-L or Ht-L will provide the desired heteroaryl ether Ht-O—Ar upon reaction with an arylboronic acid of Formula ArB(OH)₂ in the presence of a base. A variety of bases can be employed in the reaction of the compound of Formula Ht-(O)ₖ-L with the arylboronic acid. In some embodiments, the base includes or consists of a metal carbonate or bicarbonate or phosphate, for example a Group I or Group II metal carbonate or phosphate. Examples of such Group I or Group II metal carbonates and phosphates include but are not limited to Cs₂CO₃, Na₂CO₃, NaHCO₃, K₂CO₃, KHCO₃ Na₃PO₄, K₃PO₄, K₂HPO₄ and Cs₃PO₄. In some preferred embodiments, the base includes or consists of Cs₂CO₃.

In some embodiments, the reaction is performed in the presence of oxygen, for example in the presence of air, or an atmosphere containing a higher percentage by weight of oxygen than air, up to and including a pure oxygen atmosphere.

While not wishing to be bound by a particular theory, it is believed that under the reaction conditions described herein, H₂O₂ reacts with base to produce hydroperoxide ion, which performs the role of oxygen in the reaction. Accordingly, in some preferred embodiments, the reactions are performed in the presence of H₂O₂, either with or without Pd catalyst and oxygen. Typically, the H₂O₂ is employed in an amount of about 0.4% to about 0.8%, for example at about 0.8%.

In some preferred embodiments, a palladium (0) catalyst, for example Pd(PPh₃)₄, is also present in the reaction mixture. It has been discovered that the inclusion of such a catalyst significantly increases the yield of the reaction.

In some more preferred embodiments, the reaction is performed in the presence of both oxygen and a palladium (0) catalyst.

In some embodiments, the reaction of the compound of Formula Ht-(O)ₖ-L with the arylboronic acid is performed in the presence of a catalyst, which is preferably a palladium (0) catalyst. A variety of palladium (0) catalysts will find use in the present invention. These include, without limitation, one or more of bis[1,2-bis(diphenylphosphino)ethane]palladium(0), bis(dibenzylideneacetone) palladium(0), 1,3-Bis(2,6-diisopropylphenyl)imidazol-2-ylidene(1,4-naphthoquinone)palladium(0) dimer, bis(3,5,3′,5′-dimethoxydibenzylideneacetone) palladium(0), bis(tri-tert-butylphosphine)palladium(0), 1,3-bis(2,4,6-trimethylphenyl)imidazol-2-ylidene (1,4-naphthoquinone)palladium(0) dimer, tetrakis(methyldiphenyl phosphine)palladium(0), tetrakis(triphenylphosphine)palladium(0), tris(dibenzylidene acetone)dipalladium(0), tris(dibenzylideneacetone)dipalladium(0)-chloroform adduct, and tris(3,3′,3″-phosphinidynetris(benzenesulfonato)palladium(0) nonasodium salt nonahydrate. In some embodiments, tetrakis(triphenylphosphine)palladium(0); Pd(PPh₃)₄, is preferred.

Typically, the reaction of the compound of Formula Ht-(O)ₖ-L with the arylboronic acid is performed in a solvent system. The solvent system contains one or more organic solvents. A wide variety of organic solvents can be employed for the solvent systems, including polar organic solvents, preferably polar aprotic organic solvents—i.e., organic solvents that are not readily deprotonated in the presence of a strongly basic reactant. Suitable aprotic solvents can include, by way of example and without limitation, ethers, halogenated hydrocarbons (e.g., chlorinated hydrocarbons such as methylene chloride and chloroform), N,N-dimethylformamide (DMF), N,N-dimethylacetamide (DMAC), 1,3-dimethyl-3,4,5,6-tetrahydro-2(1H)-pyrimidinone (DMPU), 1,3-dimethyl-2-imidazolidinone (DMI), N-methyl-2-pyrrolidinone (NMP, or N-methyl-2-pyrrolidone), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide. Also included within the term aprotic solvent are esters, hydrocarbons, alkylnitriles, and many ether solvents including: dimethoxymethane, 1,2-dimethoxyethane, tetrahydrofuran, 1,3-dioxane, 1,4-dioxane, furan, diethyl ether, tetrahydropyran, diisopropyl ether, dibutyl ether, ethylene glycol dimethyl ether, ethylene glycol diethyl ether, diethylene glycol dimethyl ether, diethylene glycol diethyl ether, triethylene glycol dimethyl ether, anisole, and t-butyl methyl ether. In some embodiments, the reaction is performed in a solvent system that includes or consists of an ether or di-ether, for example 1,2-dimethoxyethane or tetrahydrofuran (THF). In some embodiments, the reaction is performed in a solvent system that includes a small amount of water, for example up to about 12% v/v. While not wishing to be bound by any particular theory, it is believed that inclusion of a small amount of water can be beneficial, possibly by promoting hydrolysis of base. However, the presence of greater amounts of water in the solvent system can adversely affect the yield.

Typically, the compound of Formula Ht-(O)ₖ-L is placed in solution with the arylboronic acid and the base (for example Cs₂CO₃), and the palladium (0) catalyst is added to the solution. However, that order of the addition of reagents is not critical.

The aryl boronic acid is typically employed at a molar ratio of from about 1.2 to about 10, preferably from about 1.5 to about 8, more preferably from about 2 to about 4, more preferably from about 2.2 to about 3, relative to the compound of Formula Ht-(O)ₖ-L. Typically, the base (e.g., Cs₂CO₃), is employed at a molar ratio of from about 1 to about 8, preferably from about 2 to about 6, more preferably from about 2 to about 5, more preferably from about 3 to about 5, relative to the compound of Formula Ht-(O)ₖ-L. In some embodiments, the base is employed at a molar ratio of about 4, relative to the compound of Formula Ht-(O)ₖ-L.

Preferably, the palladium (0) catalyst is employed in an amount that is sufficient to catalyze the reaction in a reasonable amount of time. In some embodiments, the palladium (0) catalyst is employed at a molar ratio of up to about 0.2%, or up to about 0.15%, or from about 0.01 to about 0.20%, or from about 0.05 to about 0.15%, or from about 0.10 to about 0.15%, relative to the compound of Formula Ht-(O)ₖ-L.

Typically, the reaction is performed at a convenient temperature, for example less than about 100° C., for example from about 0° C. to about 60° C., preferably from about room temperature (i.e. about 25° C.) to about 50° C., more preferably about 45° C. The reaction of the compound of Formula Ht-(O)ₖ-L and the arylboronic acid is typically complete after a time of from a few minutes to several days. The progress of the reaction can be monitored by any convenient method, including for example gas and liquid chromatographic techniques.

When the reaction is complete, the compound or the salt thereof can be isolated form the reaction mixture by standard work-up procedures, for example by evaporating the residue, by precipitation (followed by filtration). The compound or salt thus obtained can further be purified by any standard technique, for example by recrystallization.

As discussed above, and as shown in Reaction A of Schemes 1 and 2, the compound of Formula Ht-(O)ₖ-L can be prepared by reaction of an alcohol of Formula Ht-OH with a coupling reagent containing a phosphonium salt having a cation of Formula:

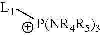

wherein:

L₁ is a moiety of Formula:

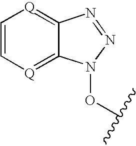

one Q is CH, and one Q is CH or N;

each R₄ and each R₅ is independently C₁₋₆ alkyl;

and wherein any R₄ and R₅ attached to the same nitrogen atom can together form a moiety of formula —(CH₂)_{q}— where q is 2, 3, 4, 5 or 6. The anion of the coupling reagent can be any of a wide variety of anions, for example halides and phosphates, for example hexafluorophosphate and halides such as bromide and chloride. One example of a coupling reagent wherein Q is CH is BOP (benzotriazol-1-yloxy)tris(dimethylamino) phosphonium hexafluorophosphate). One example of a coupling reagent wherein Q is N is PyAOP (7-azabenzotriazol-1-yloxy)tripyrrolidinophosphonium hexafluoro-phosphate).

The coupling reagent is typically employed in molar excess relative to the compound of Formula Ht-OH of about 5% or more, for example from about 10% to about 50%, or from about 10% to about 40%, or from about 20% to about 40% molar excess.

The reaction of the compound Formula Ht-OH and the coupling reagent is typically performed in the presence of a base. Any of the variety of non-nucleophilic bases that are known to be useful in coupling reaction that produce ethers are amenable to the present invention. One example of such a base is 8-diazabicyclo[5.4.0]undec-7-ene (DBU). Other suitable bases include diisopropylethylamine (DIPEA), triethylamine (TEA), and Group I and Group II metal carbonates and phosphates. The base is typically employed in molar excess relative to both the compound of Formula Ht-OH and the coupling reagent, preferably at about 5%-25% molar excess relative to the coupling reagent.

Typically, the reaction of the compound of Formula Ht-OH with the coupling reagent is performed in a solvent system. The solvent system contains one or more organic solvents. A wide variety of suitable organic solvents can be employed for the solvent systems, including polar organic solvents, preferably polar aprotic organic solvents as described above for the reaction of the compound of Formula Ht-(O)ₖ-L with the arylboronic acid. One particularly preferred solvent is acetonitrile.

As discussed above, and as shown in Schemes 1 and 2, compounds of Formula Ht-(O)ₖ-L where k is 0 can be prepared by performing the reaction under an inert atmosphere (Scheme 2, Reaction A). In such cases, it is beneficial to include a triazole compound in the reaction mixture. Typically, the triazole compound is the triazolyl moiety of the coupling reagent. For example, for reactions where BOP is used as the coupling reagent, benzotriazole is typically used as the triazole compound, and where PyAOP is used as the coupling reagent, 3H-[1,2,3]triazolo[4,5-b]pyridine is typically used as the triazole compound. Typically, the triazole compound is employed in the reaction mixture at a molar ratio relative to the compound of Formula Ht-OH of from about 2 to about 10, or from about 2 to about 5; or from about 2 to about 4; or about 3.

Typically, the compound of Formula Ht-OH is placed in solution, for example in a solvent system, with the coupling reagent, and the base (for example DBU) is added to the solution. However, that order of the addition of reagents is not critical.

Typically, the reaction is performed at a temperature of from about 0° C. to about 60° C., and conveniently at about room temperature (i.e. about 25° C.). The reaction is typically complete after a time of from a few minutes to a few hours, typically one hour, although for reactions performed in an inert atmosphere that result in compounds wherein k is 0, the reaction times can be as long as a few days, more typically from about 20 to about 40 hours, for example about 30 hours. The progress of the reaction can be monitored by any convenient method, including for example gas and liquid chromatographic techniques.

When the reaction of the compound of Formula Ht-OH with the coupling reagent is complete, the compound or the salt thereof can be isolated form the reaction mixture by standard work-up procedures, for example by evaporating the residue, by precipitation (followed by filtration). The compound or salt thus obtained can further be purified by any standard technique, for example by recrystallization.

As shown in Scheme 1, Reaction A, compounds of Formula Ht-(O)ₖ-L where k is 1 can be prepared by performing the reaction of the compound of Formula Ht-OH with the coupling reagent and base in the presence of an oxygen, for example air, or an atmosphere having an oxygen content by weight that is greater than that of air.

As shown in Scheme 2, Reaction A, compounds of Formula Ht-(O)ₖ-L where k is 0 can be prepared by performing the reaction of the compound of Formula Ht-OH with the coupling reagent and base (and preferably the triazole compound) in an inert atmosphere, for example under a nitrogen atmosphere.

In some further embodiments, the invention provides synthetic processes including the steps of:

(a) reacting a compound of Formula Ht-OH with a coupling reagent of Formula:

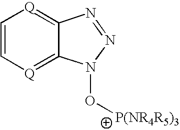

wherein:

one Q is CH, and one Q is CH or N;

each R₄ and R₅ is independently C₁₋₆ alkyl;

and wherein any R₄ and R₅ attached to the same nitrogen atom can together form a moiety of formula —(CH₂)_{q}— where q is 2, 3, 4, 5 or 6;

in the presence of a base, for example DBU, to form a compound of Formula II:

Ht-(O)ₖ-L II

or a salt thereof, wherein:

k is 0 or 1;

Ht is a heterocycle of Formula a, b, c or d:

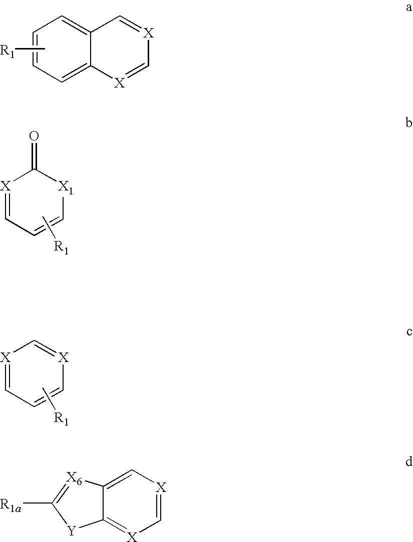

R₁, R₁ₐ and R_{1b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

each X is independently N or CH;

X₆ is CR_{1b} or N;

X₁ is NH or CH₂;

Y is S or O;

L is a group having the Formula:

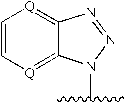

wherein one Q is CH, and one Q is CH or N; and

(b) reacting the compound of Formula II with a compound of Formula Ar—B(OH)₂;

wherein Ar has one of the Formulas e-j:

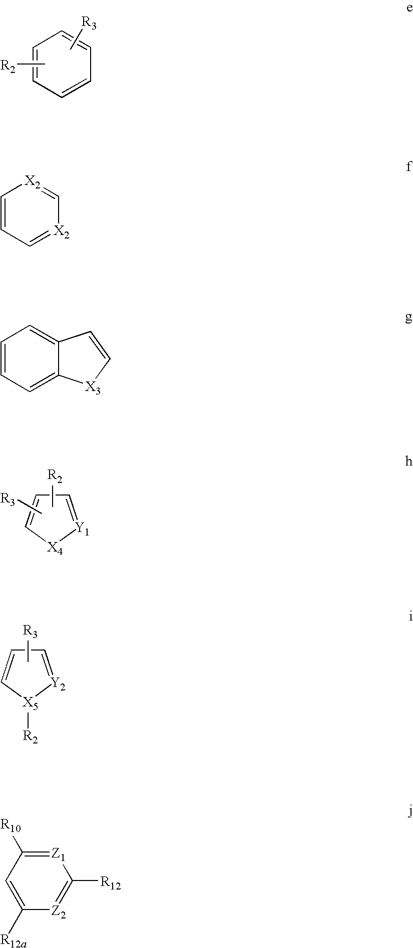

wherein:

each X₂ is independently N or CH;

X₃ is NH or CH₂;

X₄ is NH, S or O;

X₅ is N or CH;

Y₁ is N or CH;

Y₂ is N or CH;

R₂ and R₃ are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;

Z₁ is CR₁₁ₐ or N;

Z₂ is CR_{11b} or N; and

R₁₀, R₁₁ₐ, R_{11b}, R₁₂ and R₁₂ₐ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;

wherein the reaction is performed in the presence of a base;

and optionally in the presence of water;

and optionally in the presence of one or more of:
(i) oxygen;
(ii) a Pd(0) catalyst; and
(iii) H₂O₂;
   for a time and under conditions effective to form a compound of Formula Ht-O—Ar.

In some embodiments, Ht has the Formula a, wherein each X is N. In some further embodiments, Ht has the Formula d, wherein each X is N and Y is S. In some further embodiments, Ht has the Formula c, wherein each X is N. In some further embodiments, Ht has the Formula b, wherein X is N, and X₁ is NH.

In some further embodiments, Ar has the Formula e. In some further embodiments, Ar has the Formula f, wherein one X₂ is N and the other X₂ is CH. In some further embodiments, Ar has the Formula f, wherein each X₂ is N. In some further embodiments, Ar has the Formula g, wherein X₃ is NH. In some further embodiments, Ar has the Formula h, wherein X₄ is 0, and Y₁ is N. In some further embodiments, Ar has the Formula i, and in some further embodiments, Ar has the Formula j.

In some preferred embodiments, Ht has the Formula a wherein each X is N, and Ar has the Formula j.

In a further aspect, the invention provides compounds of Formula III:

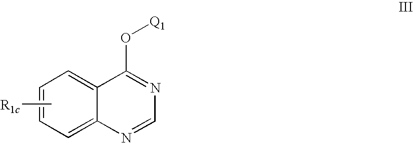

wherein:

R_{1c} is H, C₁₋₁₄ alkyl, C₁₋₁₄ alkoxy, halogen, C₇₋₂₀ arylalkyl, cyano, nitro, C₂₋₁₄ carboalkoxy, C₂₋₁₄ alkanoyl or trihaloalkyl;

Q₁ is selected from formulas j, k, m, n and o:

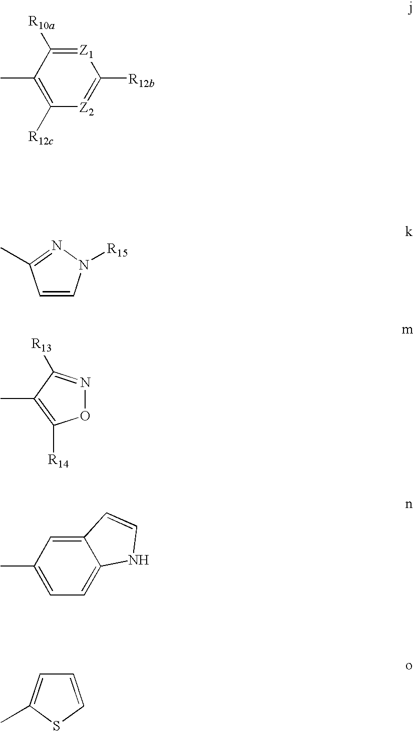

### Z₁ is CR₁₁ₐ or N;

### Z₂ is CR_{11b} or N;

R₁₀ₐ, R₁₁ₐ, R_{11b}, R_{12b} and R_{12c} are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;
R₁₃ and R₁₄ are each independently selected from the group consisting of H, C₁₋₁₄ alkyl and C₇₋₂₄ arylalkyl; and
R₁₅ is H, C₁₋₁₄ alkyl, C₇₋₂₄ arylalkyl, or C₁₋₆ trihaloalkyl;
provided that:

(i) when R₁₀ₐ and R_{12c} are each H, and Z₁ and Z₂ are each CH, then R_{12b} is not NO₂; and

(ii) when R_{12b} is H, and Z₁ and Z₂ are each CH, then neither R₁₀ₐ nor R_{12c} is NO₂.

In some embodiments, Q₁ has the Formula j, Z₁ is CR₁₁ₐ and Z₂ is CR_{11b}. In some such embodiments, Z₁ and Z₂ are each CH. In some further such embodiments, Z₁ and Z₂ are each CH and R_{12b} is H.

In some embodiments, Q₁ has the Formula j, Z₁ is CR₁₁ₐ, Z₂ is CR_{11b}, and R₁₀ₐ and R_{12b} are each H.

In some embodiments, Q₁ has the Formula j, Z₁ and Z₂ are each CH; and R₁₀ₐ and R_{12c} are each H.

In some embodiments, Q₁ has the Formula j, Z₁ is CR₁₁ₐ and Z₂ is CR_{11b}, and R_{12b} is selected from the group consisting of CF₃, —C(═O)CH₃, —C(═O)CH₂CH₃, —OCH₃, —OCH₂CH₃, —CH₃, —CH₂CH₃, CN, halogen and C₇₋₂₄ arylalkyl. In some such embodiments, Z₁ and Z₂ are each H.

In some embodiments, Q₁ has the Formula j, Z₁ and Z₂ are each CH, R₁₀ₐ and R_{12c} are each H, and R_{12b} is selected from the group consisting of CF₃, —C(═O)CH₃, —C(═O)CH₂CH₃, —OCH₃, —OCH₂CH₃, —CH₃, —CH₂CH₃, CN, halogen and C₇₋₂₄ arylalkyl.

In some embodiments, Q₁ has the Formula j, Z₁ is CR₁₁ₐ, and Z₂ is N. In some such embodiments, R_{12b} is halogen. In further such embodiments, R_{12c} is alkoxy.

In some embodiments, the invention provides compounds having the Formula IV:

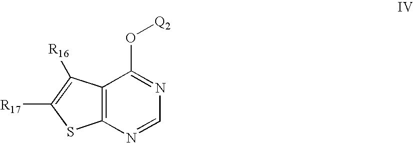

wherein:

R₁₆ and R₁₇ are each independently H, C₁₋₁₄ alkyl, carboxy, C₂₋₁₄ carboalkoxy, C(═O)CH₃, —C(═O)CH₂CH₃, C₂₋₁₄ alkanoyl or C₇₋₂₄ arylalkyl; and

Q₂ has the Formula:

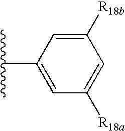

R₁₈ₐ and R_{18b} are each independently H, C₁₋₁₄ alkyl, C₁₋₁₄ alkoxy, halogen, C₇₋₂₄ arylalkyl, cyano, nitro, C₂₋₁₄-carboalkoxy, C₂₋₁₄ alkanoyl, C₁₋₆ trihaloalkyl, N(R₅₀)(R₅₁), —NH₂, —NH(C₁-C₆ alkyl), —N(C₁-C₆ alkyl)(C₁-C₆ alkyl), —N(C₁-C₃ alkyl)C(O)(C₁-C₆ alkyl), —NHC(O)(C₁-C₆ alkyl), —NHC(O)H, —C(O)NH₂, —C(O)NH(C₁-C₆ alkyl), —C(O)N(C₁-C₆ alkyl)(C₁-C₆ alkyl), —CN, —OH, —C(O)OC₁-C₆ alkyl, —C(O)C₁-C₆ alkyl, C₆-C₁₄ aryl or C₄-C₁₀ heteroaryl;

R₅₀ and R₅₁ are each independently H, C₁₋₁₄ alkyl, C₂₋₁₄ alkenyl, C₂₋₁₄ alkynyl, C₇₋₂₄ arylalkyl, C₂₋₁₄ alkanoyl, C₁₋₆ trihaloalkyl, —S(O)ᵥ—C₁₋₁₄ alkyl; —S(O)ᵥ—C₆₋₁₄ aryl, —S(O)ᵥ—C₇₋₂₄ arylalkyl or —S(O)ᵥ—C₇₋₂₄ alkylaryl; where v is 0, 1 or 2;

or R₅₀ and R₅₁ together can form a moiety of formula —(CH₂)ᵣ— where r is 2, 3, 4, 5 or 6;

or Q₂ has the Formula m:

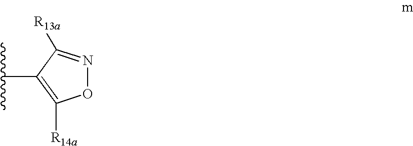

where R₁₃ₐ and R₁₄ₐ are each independently selected from the group consisting of H, C₁₋₁₄ alkyl and C₇₋₂₄ arylalkyl.

In some embodiments, Q₂ has the Formula:

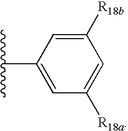

In some such embodiments, R₁₈ₐ and R_{18b} are each independently selected from the group consisting of H, C₁₋₁₄ alkyl, C₁₋₁₄ alkoxy, halogen, C₇₋₂₄ arylalkyl, cyano, nitro, C₂₋₁₄-carboalkoxy, C₂₋₁₄ alkanoyl and C₁₋₆ trihaloalkyl.

In some embodiments, Q₂ has the Formula m:

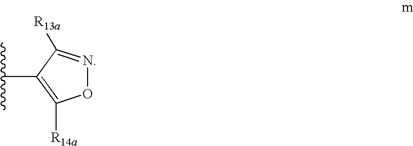

In some such embodiments, R₁₃ₐ and R₁₄ₐ are each C₁₋₁₄ alkyl.

In some embodiments, the invention further provides compounds of Formula V:

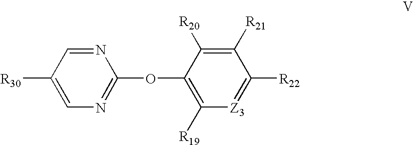

wherein:

### Z₃ is N or CR_{11c};

R_{11c} is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₁₉ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₀ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₁ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₂ is H F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl; and
R₃₀ is halogen;
provided that:

(i) when Z₃ is CR_{11c}, then at least one of R₁₉, R₂₀, R₂₁ and R₂₂ is other than H; and

(ii) when Z₃ is N, the R_{11c}, R₁₉, R₂₀, R₂₁ and R₂₂ are each independently selected from H, C₂₋₁₄ alkenyl, —S—C₁₋₁₄ alkyl and C₁₋₁₄ alkoxy.

In some embodiments, Z₃ is N. In some such embodiments, R₃₀ is bromine. In some such embodiments, R₁₉ is C₁₋₁₄ alkoxy. In some such embodiments, R₂₀, R₂₁ and R₂₂ are each H.

In some embodiments, Z₃ is CR_{11c}. In some such embodiments, R_{11c}, R₁₉, R₂₀, R₂₁ and R₂₂ are each H.

In some embodiments, the invention further provides compounds of Formula VI:

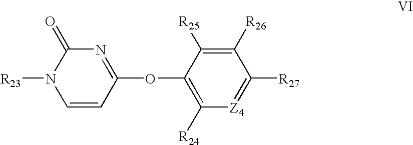

wherein:

### (A) Z₄ is N;

R₂₃ is C₁₋₁₄ alkyl or C₇₋₂₄ arylalkyl; and

R₂₄, R₂₅, R₂₆ and R₂₇ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;

or

### (B) Z₄ is CR_{11d};

R_{11d} is H or C₁₋₁₄ alkyl;

R₂₃ is C₁₋₁₄ alkyl; and

R₂₄, R₂₅, R₂₆ and R₂₇ are each independently selected from the group consisting of H, C₁₋₁₄ alkoxy, C₇₋₂₄ arylalkyl, cyano, C₂₋₁₄ carboalkoxy, C₂₋₁₄ alkanoyl and C₁₋₆ trihaloalkyl.

In some embodiments, Z₄ is N. In some such embodiments, R₂₄ is C₁₋₁₄ alkoxy. In further such embodiments, R₂₄, R₂₅, R₂₆ and R₂₇ are each independently selected from H, C₁₋₁₄ alkoxy, cyano and C₁₋₆ trihaloalkyl.

In some embodiments wherein Z₄ is N, R₂₄, R₂₅, R₂₆ and R₂₇ are each independently selected from H, OCH₃, cyano and CF₃.

In some embodiments, Z₄ is CR_{11d}. In some such embodiments, R₂₄ is C₁₋₁₄ alkoxy. In some such embodiments, R₂₃ is methyl; and R₂₄ is methoxy.

In some embodiments, the invention provides compounds of Formula XXX:

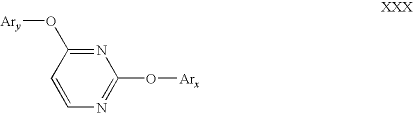

wherein:

Arₓ is phenyl or pyridyl, each of which is optionally substituted with up to 3 substituents selected from F, Cl, Br, I, C₁₋₁₄ alkyl, C₁₋₁₄ alkoxy, C₁₋₁₄ alkanoyl, NO₂, and C₂₋₁₄ carboalkoxy; and

Ar_{y} is phenyl or pyridyl, each of which is optionally substituted with up to 3 substituents selected from F, Cl, Br, I, C₁₋₁₄ alkyl, C₁₋₁₄ alkoxy, C₁₋₁₄ alkanoyl, NO₂, and C₂₋₁₄ carboalkoxy;

or Ar_{y} is:

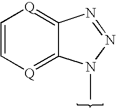

where one Q is CH, and one Q is CH or N.

In some further embodiments, the invention provides compounds of Formula XXXI:

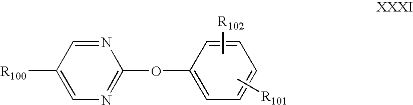

wherein:

R₁₀₀ is phenyl, optionally substituted with 1 or 2 substituents independently selected from F, Cl, Br, I, C₁₋₁₄ alkyl, C₁₋₁₄ alkoxy, C₁₋₁₄ alkanoyl, NO₂, and C₂₋₁₄ carboalkoxy; and

R₁₀₁ and R₁₀₂ are each independently selected from F, Cl, Br, I, C₁₋₁₄ alkyl, C₁₋₁₄ alkoxy, C₁₋₁₄ alkanoyl, NO₂, and C₂₋₁₄ carboalkoxy.

It has been discovered in accordance with some embodiments of the invention that asymmetric 2,4-diaryl or heteroaryl pyrimidinyl ethers can be prepared by utilizing the selectivity of the coupling reactions described herein toward 2,4-di-OPT pyrimidine. As shown in Tables 5 (examples 114-125) and 6 (examples 126-129), infra, and their accompanying synthetic schemes, reaction of 2,4-di-OPT pyrimidine with arylboronic acid results in monosubstitution at the 2-position of the pyrimidine, whether coupling is performed with H₂O₂ or with O₂/Pd catalyst, while Table 7 (examples 130-134) shows that coupling performed without O₂, H₂O₂ or Pd catalyst is non-selective, producing di-ethers. Accordingly, in a further embodiment of the present invention, methods are provided for the preparation of asymmetric 2,4-diaryl, or 2-4-heteroaryl, or 2,4-mixed aryl-heteroaryl pyrimidinyl ethers, wherein 2,4-di-OPT pyrimidine is employed in place of the compound of Formula II in the methods of the invention to provide 2-(heretoaryl or aryl)ether-4-OPT compounds as shown in Tables 5 (examples 114-125) and 6 (examples 126-129), infra, and the products then reacted as shown in Examples 138-138 (Table 8) and its accompanying synthetic scheme, to provide asymmetric pyrimidine-2,4-di-(aryl or heteroaryl)ethers.

The invention further provides methods for treating an oncological disease or disorder, comprising administering to a patient in need thereof a therapeutically effective amount of a compound of the invention. The invention further provides methods for treating inflammation, comprising administering to a patient in need thereof a therapeutically effective amount of a compound of the invention.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used in this application, the term “optionally substituted,” as used herein, means that substitution is optional and therefore it is possible for the designated atom or moiety to be unsubstituted. In the event a substitution is desired then such substitution means that the indicated number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then up to 3 hydrogens on the carbon atom can be replaced.

The carbon number, as used in the definitions herein, refers to carbon backbone and carbon branching, but does not include carbon atoms of substituents, such as alkoxy substitutions and the like.

As used herein, the term “alkyl” is meant to refer to a monovalent or divalent saturated hydrocarbon group which is straight-chained or branched. Examples of alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. An alkyl group can contain from 1 to about 20, from 2 to about 20, from 1 to about 14, from 1 to about 10, from 1 to about 8, from 1 to about 6, from 1 to about 4, or from 1 to about 3 carbon atoms, or if a specified number of carbon atoms is provided then that specific number would be intended. For example “C₁₋₆ alkyl” denotes alkyl having 1, 2, 3, 4, 5 or 6 carbon atoms. As used herein, the term “lower alkyl” is intended to mean alkyl groups having up to six carbon atoms.

As used herein, “alkenyl” refers to an alkyl group having one or more carbon-carbon double bonds. Nonlimiting examples of alkenyl groups include ethenyl, propenyl, and the like.

As used herein, “alkynyl” refers to an alkyl group having one or more carbon-carbon triple bonds. Nonlimiting examples of alkynyl groups include ethynyl, propynyl, and the like.

As used herein, “aromatic” refers to having the characters such as 4n+2 delocalized electrons in a ring structure and planar configuration of the ring.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be a polycyclic moiety in which at least one carbon is common to any two adjoining rings therein (for example, the rings are “fused rings”), for example naphthyl. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls or cycloalkynyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, “cycloalkyl” refers to non-aromatic cyclic hydrocarbons including cyclized alkyl, alkenyl, and alkynyl groups, having the specified number of carbon atoms (wherein the ring comprises 3 to 20 ring-forming carbon atoms). Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like, or any subset thereof. Also included in the definition of cycloalkyl are moieties that have one or more aromatic rings fused (i.e., having a bond in common with) to the cycloalkyl ring, for example, benzo derivatives of cyclopentane (i.e., indanyl), cyclopentene, cyclohexane, and the like. The term “cycloalkyl” further includes saturated ring groups, having the specified number of carbon atoms. These may include fused or bridged polycyclic systems. Suitable cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, the term “heterocyclyl” or “heterocyclic” or “heterocycle” refers to ring-containing monovalent and divalent structures having one or more heteroatoms, independently selected from N, O and S, as part of the ring structure and comprising from 3 to 20 atoms in the rings, or 3- to 7-membered rings. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H, 6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiirane, triazinyl, 1,2,3-triazolyl, 1,2,4-triazolyl, 1,2,5-triazolyl, 1,3,4-triazolyl, and xanthenyl, or any subset thereof.

As used herein, “heteroaryl” refers to an aromatic heterocycle (wherein the ring comprises up to about 20 ring-forming atoms) having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like, or any subset thereof. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “heterocycloalkyl” refers to non-aromatic heterocycles (wherein the ring comprises about 3 to about 20 ring-forming atoms) including cyclized alkyl, alkenyl, and alkynyl groups where one or more of the ring-forming carbon atoms is replaced by a heteroatom such as an O, N, or S atom. Heterocycloalkyl groups can be mono or polycyclic (e.g., fused-, bridged- and spiro-systems). Suitable “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy, or any subset thereof. Similarly, “alkylthio” or “thioalkoxy” represent an alkyl group as defined above with the indicated number of carbon atoms attached through a sulphur bridge.

As used herein, “halo” or “halogen” includes fluoro, chloro, bromo, and iodo, or any subset thereof.

As used herein, “haloalkyl” refers to an alkyl group having one or more halogen substituents. Example haloalkyl groups include CF₃, C₂F₅, CH₂CF₃, CHF₂, CCl₃, CHCl₂, C₂Cl₅, and the like, or any subset thereof. The term “perhaloalkyl” is intended to denote an alkyl group in which all of the hydrogen atoms are replaced with halogen atoms. One example of perhaloalkyl is CH₃ or CF₃. The term “perfluoroalkyl” is intended to denote an alkyl group in which all of the hydrogen atoms are replaced with fluorine atoms. One example of perhaloalkyl is CF₃ (i.e., trifluoromethyl).

As used herein, the term “haloalkoxy” refers to an —O-haloalkyl group. An example haloalkoxy group is OCF₃.

As used herein, the term “arylalkyl” refers to an alkyl group that has an appended aryl group. Examples of arylalkyl groups include benzyl, 2-phenylethyl, naphthylmethyl and 2-naphthylethyl groups.

As used herein, the term “alkylaryl” refers to an aryl group that has an appended alkyl group. Examples of alkylaryl groups include 2-methylphenyl, 3-butylphenyl, 4-methylnaphthyl and 2-ethylnaphthyl groups.

As used herein, the term “carboalkoxy” refers to a group of formula —C(═O)—O-alkyl.

As used herein, the term “alkanoyl” refers to a group of formula —C(═O)-alkyl.

As used herein, the term “reacting” refers to the bringing together of designated chemical reactants such that a chemical transformation takes place generating a compound different from any initially introduced into the system. Reacting can take place in the presence or absence of solvent.

The compounds of the present invention can contain an asymmetric atom, and some of the compounds can contain one or more asymmetric atoms or centers, which can thus give rise to optical isomers (enantiomers) and diastereomers. The present invention includes such optical isomers (enantiomers) and diastereomers (geometric isomers), as well as, the racemic and resolved, enantiomerically pure R and S stereoisomers, as well as, other mixtures of the R and S stereoisomers and pharmaceutically acceptable salts thereof. Optical isomers can be obtained in pure form by standard procedures known to those skilled in the art, and include, but are not limited to, diastereomeric salt formation, kinetic resolution, and asymmetric synthesis. It is also understood that this invention encompasses all possible regioisomers, and mixtures thereof, which can be obtained in pure form by standard separation procedures known to those skilled in the art, and include, but are not limited to, column chromatography, thin-layer chromatography, and high-performance liquid chromatography.

Compounds of the invention can also include all isotopes of atoms occurring in the intermediates or final compounds. Isotopes include those atoms having the same atomic number but different mass numbers. For example, isotopes of hydrogen include tritium and deuterium.

Compounds of the invention can also include tautomeric forms, such as keto-enol tautomers. Tautomeric forms can be in equilibrium or sterically locked into one form by appropriate substitution.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography (TLC).

Upon carrying out preparation of compounds according to the processes described herein, the usual isolation and purification operations such as concentration, precipitation, filtration, extraction, solid-phase extraction, recrystallization, chromatography, and the like may be used to isolate the desired products.

The compounds and compositions of the present invention are useful for the prevention or treatment of oncological diseases or disorders, including benign and malignant tumors/neoplasia including cancers such as colorectal cancer, brain cancer, bone cancer, epithelial cell-derived neoplasia (epithelial carcinoma) such as basal cell carcinoma, adenocarcinoma, gastrointestinal cancer, including lip cancer, mouth cancer, esophogeal cancer, small bowel cancer and stomach cancer, colon cancer, liver cancer, bladder cancer, pancreatic cancer, ovarian cancer, cervical cancer, lung cancer, breast cancer, and skin cancers, such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreas cancer, ovarian cancer, prostatic cancer, cervical cancer, lung cancer, breast cancer, and skin cancer, such as squamous cell and basal cell cancers. Further neoplasia disorders include acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choroid plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial. squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

The compounds of the invention are further useful for treating inflammation and inflammatory diseases, including inflammation in such diseases as arthritis, colitis, encephalitis, hepatitis, pancreatitis, vascular diseases, migraine headaches, periarteritis nodosa, thyroiditis, aplastic anemia, Hodgkin's disease, scleredoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like. Also included are treatments of ophthalmic diseases, such as retinitis, conjunctivitis, retinopathies, uveitis, ocular photophobia, and of acute injury to the eye tissue. The compounds of this invention will be useful in the treatment of post-operative inflammation including that following ophthalmic surgery such as cataract surgery or refractive surgery. Also included are treatments of pulmonary and upper respiratory tract inflammation, such as that associated with viral infections and cystic fibrosis, and in bone resorption such as that accompanying osteoporosis.

In accordance with the invention, methods are provided for treating inflammation, or inflammatory disorders, or inflammatory diseases, or oncological diseases and/or disorders, including those specifically listed above, comprising the administration to a mammal in need thereof a compound of the invention, or a pharmaceutically acceptable salt thereof. Each of such methods of this invention comprises administering to a mammal in need of such treatment a pharmaceutically or therapeutically effective amount of a compound of this invention. In the instances of combination therapies described herein, it will be understood the administration further includes a pharmaceutically or therapeutically effective amount of the second pharmaceutical agent in question. The second or additional pharmacological agents described herein may be administered in the doses and regimens known in the art.

The compounds of this invention may also be used in comparable veterinary methods of treatment, particularly for the veterinary treatment, inhibition or alleviation of inflammation and pain. These methods will be understood to be of particular interest for companion mammals, such as dogs and cats, and for use in farm mammals, such as cattle, horses, mules, donkeys, goats, hogs, sheep, etc. These methods may be used to treat the types of inflammation and pain experienced in veterinary medicine including, but not limited to, pain and inflammation associated with arthritis, joint imperfections, developmental joint defects, such as hip dysplasia, tendonitis, suspensary ligament inflammation, laminitis, curb and bursitis, or pain or inflammation associated with surgery, accident, trauma or disease, such as Lyme Disease. These compounds may also be used in the treatment of inflammation of the air passages, such as in conditions of asthma, laryngitis, tracheitis, bronchitis, rhinitis and pharyngitis

Methods of treating the diseases and syndromes listed herein are understood to involve administering to an individual in need of such treatment a therapeutically effective amount of the salt form or solid dispersion of the invention, or composition containing the same. As used herein, the term “treating” in reference to a disease is meant to refer to preventing, inhibiting and/or ameliorating the disease.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds (including the salts) and the compositions of the present invention may be given at an oral dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the active compounds (including the salts) and the compositions of the present invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds (including the salts) and the compositions of the present invention directly to the airways in the form of an aerosol.

The compounds (including the salts) and the compositions of the present invention may also be administered parenterally or intraperitoneally. Solutions or suspensions of these active compounds (including the salts) and the compositions of the present invention can be prepared in water optionally mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Preparation of Representative Compounds of the Invention

Examples 1-10 show preparation of representative compounds by the procedure of Scheme 3 below.

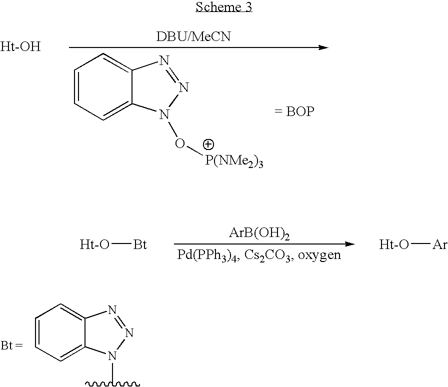

### Example 1

### 4-(1H-Benzo[d][1,2,3]triazol-1-yloxy)quinazoline

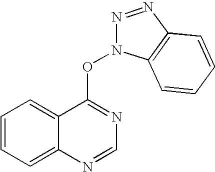

To a solution of 4-hydroxyquinazoline (730 mg, 5 mmol) and BOP (2.6 g, 6 mmol) in MeCN (40 mL) was added DBU (1.13 mL, 7.5 mmol) at room temperature. The resultant mixture was stirred for 1 hour at room temperature. The solvent was removed under vacuum, the crude mixture was purified by a flash chromatography on SiO₂ column eluted with EtOAc/hexane (1:1) to give the desired product 1.08 g (84%).

¹H NMR (DMSO-d6, 400 MHz): δ (ppm) 8.76 (s, 1H), 8.60 (d, J=4.0 Hz, 1H), 8.25-8.16 (m, 3H), 8.00 (t, J=8.0 Hz, 1H), 7.89 (d, J=8.0 Hz, 1H), 7.67 (t, J=8.0 Hz, 1H), 7.58 (t, J=8.0 Hz, 1H). ¹³C NMR (DMSO-d6, 400 MHz): 165.7, 153.2, 151.9, 143.1, 136.1, 129.6, 128.8, 128.1, 125.7, 123.0, 120.3, 113.1, 110.0. HRMS (ES-MS) [(M+H)⁺]: for C₁₄H₉N₅O 264.0879, found 264.0879.

### Example 2

### 4-(1H-Benzo[d][1,2,3]triazol-1-yloxy)thieno[2,3-d]pyrimidine

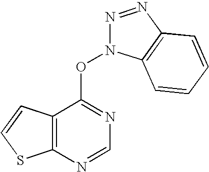

This compound was synthesized according to Example 1 from thieno[2,3-d]pyrimidin-4(3H)-one (152 mg, 1.00 mmol), BOP (520 mg, 1.20 mmol) and DBU (0.19 mL, 1.30 mmol) in MeCN (10 mL). Purified by flash chromatography as a white solid (250 mg, 93%).

¹H NMR (DMSO-d6, 400 MHz): δ (ppm) 8.64 (s, 1H), 8.23-8.22 (m, 2H), 7.91 (d, J=6.0 Hz, 1H), 7.86 (d, J=8.4 Hz, 1H), 7.66-7.56 (m, 2H). ¹³C NMR (DMSO-d6, 400 MHz): 170.6, 162.5, 152.6, 143.1, 130.3, 129.7, 128.8, 125.7, 120.2, 117.9, 116.3, 109.9. HRMS (ES-MS) [(M+H)⁺]: for C₁₂H₇N₅OS 270.0441, found 270.0441.

### Example 3

### Methyl 4-(1H-Benzo[d][1,2,3]triazol-1-yloxy)-5-methylthieno[2,3-d]pyrimidine-6-carboxylate

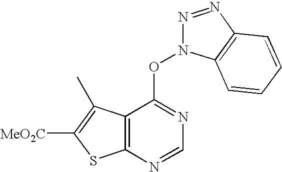

This compound was synthesized according to Example 1 from methyl 5-methyl-4-oxo-3,4-dihydrothieno[2,3-d]pyrimidine-6-carboxylate (224 mg, 1.00 mmol), BOP (520 mg, 1.20 mmol) and DBU (0.19 mL, 1.30 mmol) in MeCN (10 mL). Purified by flash chromatography as a yellow solid (260 mg, 76%).

¹H NMR (CDCl₃, 400 MHz): δ (ppm) ¹H NMR (DMSO-d6, 400 MHz): δ (ppm) 8.71 (s, 1H) 8.21 (dd, J=0.8, 7.6 Hz, 1H), 7.92-7.90 (m, 1H), 7.67-7.56 (m, 2H), 3.96 (s, 3H), 3.10 (s, 3H). ¹³C NMR (DMSO-d6, 400 MHz): 169.0, 164.7, 162.3, 155.0, 143.0, 138.7, 129.6, 128.7, 126.5, 125.8, 120.2, 118.3, 110.1, 53.2, 16.0.

HRMS (ES-MS) [(M+H)⁺]: for C₁₅H₁₁N₅O₃S 345.0655, found 345.0658.

### Example 4

### 1-(4-Methyl pyrimidin-2-yloxy)-1H-benzo[d][1,2,3]triazole

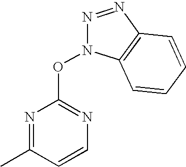

This compound was synthesized according to Example 1 from 4-methylpyrimidin-2(1H)-one (146 mg, 1.00 mmol), BOP (520 mg, 1.20 mmol) and DBU (0.45 mL, 3.00 mmol) in MeCN (10 mL). Purified by flash chromatography as a white solid (160 mg, 70%).

¹H NMR (DMSO-d6, 400 MHz): δ (ppm) 8.53 (dd, J=4.8 Hz, 1H) 8.17 (dd, J=8.4 Hz, 1H), 7.75 (dd, J=8.4 Hz, 1H), 7.64-7.53 (m, 2H), 7.42 (dd, J=5.2 Hz, 1H), 3.3 (s, 3H). ¹³C NMR (DMSO-d6, 400 MHz): 171.9, 164.1, 159.8, 142.7, 129.0, 128.0, 125.0, 119.7, 119.2, 109.2, 23.4.

HRMS (ES-MS) [(M+H)⁺]: for C₁₁H₉N₅OS 228.0879, found 228.0877.

### Example 5

### 1-(6-Chloropyrimidin-4-yloxy)-1H-benzo[d][1,2,3]tri azole

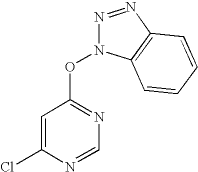

This compound was synthesized according to Example 1 from 6-chloropyrimidin-4(3H)-one (131 mg, 1.00 mmol), BOP (520 mg, 1.20 mmol) and DBU (0.19 mL, 1.30 mmol) in MeCN (10 mL). Purified by flash chromatography as a white solid (125 mg, 51%)

¹H NMR (DMSO-d6, 400 MHz): δ (ppm) 8.7 (s, 1H), 8.19 (d, J=8.4 Hz, 1H), 8.05-8.04 (m, 1H), 7.82 (d, 1H), 7.69-7.66 (m, 1H), 7.57-7.55 (m, 1H). ¹³C NMR (DMSO-d6, 400 MHz): 169.8, 162.3, 158.9, 129.8, 143.0, 128.4, 125.8, 120.3, 109.7, 107.0.

HRMS (ES-MS) [(M+H)⁺]: for C₁₀H₆ClN₆O 248.0333, found 248.0333.

### Example 6

### 4,6-Bis(1H-Benzo[d][1,2,3]triazol-1-yloxy)pyrimidine

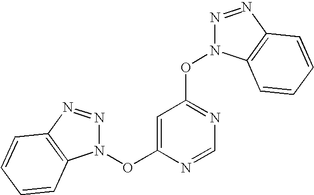

This compound was synthesized according to Example 1 from 6-chloropyrimidin-4(3H)-one (131 mg, 1.00 mmol), BOP (520 mg, 1.20 mmol) and DBU (0.19 mL, 1.30 mmol) in MeCN (10 mL). Purified by flash chromatography as a white solid (110 mg, 31%).

¹H NMR (DMSO-d6, 400 MHz): δ (ppm) 8.45 (d, J=0.8 Hz, 1H), 8.20 (d, J=8.4 Hz, 2H), 7.87-7.86 (m, 2H), 7.84 (s, 1H), 7.71-7.67 (m, 2H), 7.58-7.56 (m, 2H). ¹³C NMR (DMSO-d6, 400 MHz): 171.3, 158.7, 143.0, 129.8, 128.5, 125.8, 120.3, 109.7, 90.7. HRMS (ES-MS) [(M+H)⁺]: for C₁₆H₁₀N₈O₂ 347.0999, found 347.1005.

### Example 7

### 4-Phenoxy-quinazoline

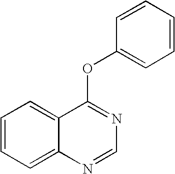

To a solution of 4-(1H-benzo[d][1,2,3]triazol-1-yloxy)quinazoline (Example 1) (50 mg, 0.19 mmol), phenyl boronic acid (54 mg, 0.42 mmol) and Cs₂CO₃ (0.76 mmol, 247 mg) in DME (2 mL) was added Pd(PPh₃)₄ (32 mg). Then the reaction mixture was stirred under oxygen atmosphere for 10 h at 45° C. Then solvent was removed by rotavap and residue was purified by silica gel chromatography (20% EtOAc/Hex) to give desired product (31 mg, 74%).

¹H NMR (DMSO-d6, 400 MHz): δ (ppm) 8.85 (s, 1H), 8.26 (d, J=8.2 Hz, 1H), 8.05-8.00 (m, 2H), 7.80 (t, J=6.3 Hz, 1H), 7.68-7.66 (m, 2H), 7.56-7.54 (m, 3H). ¹³C NMR (DMSO-d6, 400 MHz): 170.6, 153.8, 148.1, 136.1, 134.9, 130.2, 129.9, 128.8, 128.7, 126.8, 123.9, 122.7.

HRMS (ES-MS) [(M+H)⁺]: for C₁₄H₁₀N₂O₁ 223.0861, found 223.0862.

### Example 8

### 4-Phenoxy-quinazoline

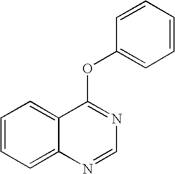

To a solution of 4-(1H-benzo[d][1,2,3]triazol-1-yloxy)quinazoline (50 mg, 0.19 mmol), phenyl boronic acid (54 mg, 0.42 mmol) and Cs₂CO₃ (0.76 mmol, 247 mg) in DME (2 mL) was added Pd(PPh₃)₄ (32 mg). Then the reaction mixture was stirred under air for 10 h at 60° C. under air atmosphere. Then solvent was removed by rotavap and residue was purified by silica gel chromatography (20% EtOAc/Hex) to give desired product (25 mg, 60%).

¹H NMR (DMSO-d6, 400 MHz): δ (ppm) 8.85 (s, 1H), 8.26 (d, J=8.2 Hz, 1H), 8.05-8.00 (m, 2H), 7.80 (t, J=6.3 Hz, 1H), 7.68-7.66 (m, 2H), 7.56-7.54 (m, 3H). ¹³C NMR (DMSO-d6, 400 MHz): 170.6, 153.8, 148.1, 136.1, 134.9, 130.2, 129.9, 128.8, 128.7, 126.8, 123.9, 122.7.

HRMS (ES-MS) [(M+H)⁺]: for C₁₄H₁₀N₂O₁ 223.0861, found 223.0862.

### Example 9

### 4-(4-(Trifluoromethyl)phenoxy)quinazoline

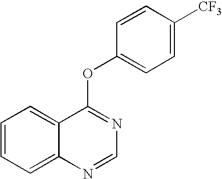

This compound was synthesized according to Example 8 from 4-(1H-benzo[d][1,2,3]triazol-1-yloxy)quinazoline (50 mg, 0.19 mmol), phenyl boronic acid (54 mg, 0.42 mmol), Pd(PPh₃)₄ (32 mg) and Cs₂CO₃ (0.76 mmol, 247 mg) in DME (2 mL) The reaction mixture was then stirred under air for 10 h at 60° C. under air atmosphere and purified by flash chromatography as a white solid (37 mg, 60%).

¹H NMR (DMSO-d6, 400 MHz): δ (ppm) 8.76 (s, 1H), 8.40 (dd, J=0.4, 8.4 Hz, 1H), 8.10-8.02 (m, 2H), 7.90 (d, J=8.8 Hz, 2H), 7.84-7.80 (m, 1H), 7.64 (d, J=8.8 Hz, 2H). ¹³C NMR (DMSO-d6, 400 MHz): 166.3, 155.2, 153.9, 151.5, 135.1, 128.5, 127.9, 127.5, 127.4, 123.7, 123.5, 115.8.

HRMS (ES-MS) [(M+H)⁺]: for C₁₅H₉N₂OF₃ 231.0739, found 291.0745.

### Example 10

### 1-(4-(Quinazolin-4-yloxy)phenyl)ethanone

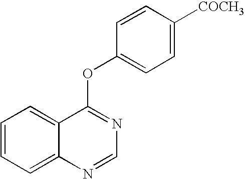

This compound was synthesized according to Example 8 from 4-(1H-benzo[d][1,2,3]triazol-1-yloxy)quinazoline (50 mg, 0.38 mmol)), phenyl boronic acid (54 mg, 0.42 mmol), Pd(PPh₃)₄ (32 mg) and Cs₂CO₃ (0.76 mmol, 247 mg) in DME (2 mL). The reaction mixture was then stirred under air for 10 h at 60° C. under air atmosphere and then purified by flash chromatography as a white solid (28 mg, 56

¹H NMR (CDCl₃, 400 MHz) δ (ppm) 8.78 (s, 1H), 8.38 (d, J=8.0 Hz, 1H), 8.11 (d, J=8.8 Hz, 2H), 8.51-8.31 (m, 1H), 7.95 (t, J=5.0 Hz, 1H), 7.70 (t, J=5.0 Hz, 1H), 7.38 (d, J=8.8 Hz, 2H), 2.64 (s, 3H). ¹³C NMR (CDCl₃, 100 MHz): δ (ppm) 197.1, 166.8, 156.5, 154.3, 152.1, 132.2, 134.7, 130.6, 128.3, 128.2, 123.8, 122.74, 116.6, 27.0.

HRMS (ES-MS) [(M+H)⁺]: for C₁₆H₂N₂O₂ 265.0971, found 265.0968.

Examples 11-62 show preparation of representative compounds by the procedure of Scheme 4 below.

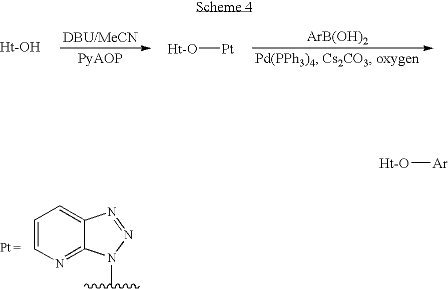

### Example 11

### 4-(3H-[1,2,3]Triazolo[4,5-b]pyridin-3-yloxy)quinazoline

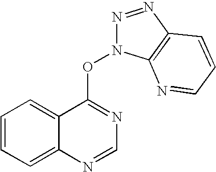

To a solution of 4-hydroxyquinazoline (2.94 g, 20 mmol) and PyAOP (11.4 g, 22 mmol) in MeCN (100 mL) was added DBU (5.2 mL, 30 mmol) at room temperature. The resultant mixture was stirred for 1 hour at room temperature. The solvent was removed under vacuum, the crude mixture was purified by a flash chromatography on SiO₂ column eluted with EtOAc/hexane (4:1) to give the desired product 2.87 g (80%).

¹H NMR (CDCl₃, 400 MHz): δ (ppm) 8.73 (dd, J=1.2, 4.4 Hz, 1H), 8.64 (s, 1H), 8.53 (dd, J=1.2, 8.0 Hz, 2H), 8.14-8.11 (m, 1H), 8.07-8.03 (m, 1H), 7.84-7.08 (m, 1H), 7.50 (dd, J=4.4, 8 Hz, 1H). ¹³C NMR (CDCl₃, 100 MHz): δ (ppm) 156.8, 152.9, 152.2, 151.8, 142.1, 135.1, 135.1, 129.7, 128.7, 128.2, 122.6, 121.0, 113.6.

HRMS (ES-MS) [(M+H)⁺]: for C₁₃H₈N₆O 265.0832, found 265.0836.

### Example 12

### 4-(3H-[1,2,3]Triazolo[4,5-b]pyridin-3-yloxy)thieno[2,3-d]pyrimidine

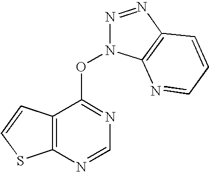

This compound was synthesized according to Example 11 from thieno[2,3-d]pyrimidin-4(3H)-one (608 mg, 4.00 mmol), PyAOP (2.28 g, 4.40 mmol) and DBU (0.7 mL, 4.80 mmol) in MeCN (30 mL) and was purified by flash chromatography as a white solid (980 mg, 88%).

¹H NMR (CDCl₃, 400 MHz): δ (ppm) 8.73 (dd, J=1.2, 4.4 Hz, 1H), 8.61 (s, 1H), 8.51 (dd, J=1.2, 8.0 Hz, 1H), 8.14 (s, 1H), 7.67 (s, 1H), 7.50 (dd, J=4.8, 8.4 Hz, 1H). ¹³C NMR (CDCl₃, 100 MHz): δ (ppm) 164.6, 163.5, 153.5, 151.9, 140.8, 136.9, 134.9, 129.7, 124.6, 121.0, 114.6.

MS (ES-MS) [(M+H)⁺]: for C₁₁H₁₆N₆ONS 271.2, found 271.2.

### Example 13

### Methyl 4-(3H-[1,2,3]triazolo[4,5-b]pyridin-3-yloxy)-5-methylthieno[2,3-d]pyrimidine-6-carboxylate

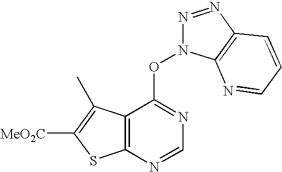

This compound was synthesized according to Example 11 from methyl 5-methyl-4-oxo-3,4-dihydrothieno[2,3-d]pyrimidine-6-carboxylate (950 mg, 4.00 mmol), PyAOP (2.28 g, 4.40 mmol) and DBU (0.7 mL, 4.80 mmol) in MeCN (30 mL) and was purified by flash chromatography as a yellow solid (1.25 g, 91%).

¹H NMR (CDCl₃, 400 MHz): δ (ppm) 8.72 (dd, J=0.80, 4.0 Hz, 1H), 8.52 (dd, J=1.2, 8.0 Hz. 1H), 8.49 (s, 1H), 7.50 (dd, J=4.0, 8.1 Hz, 1H), 4.00 (s, 3H), 3.19 (s, 3H). ¹³C NMR (CDCl₃, 100 MHz): δ (ppm) 170.0, 164.6, 162.5, 154.1, 151.9, 140.8, 138.3, 135.0, 129.7, 127.5, 121.0, 118.1, 52.7, 15.9.

MS (ES-MS) [(M+H)⁺]: for C₁₄H₁₀N₆O₃NS 343.2, found 343.2.

### Example 14

### 3-(5-Bromopyrimidin-2-yloxy)-3H-[1,2,3]triazolo[4,5-b]pyridine

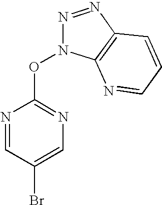

This compound was synthesized according to Example 11 from 5-bromopyrimidin-2(1H)-one (173 mg, 1.00 mmol), PyAOP (520 mg, 1.10 mmol) and DBU (0.17 mL, 1.20 mmol) in MeCN (10 mL) and was purified by flash chromatography as a white solid (210 mg, 75%).

¹H NMR (DMSO-d6, 400 MHz): δ (ppm) 8.96 (s, 2H), 8.81 (dd, J=1.2, 4.4 Hz, 1H), 8.75 (dd, J=0.80, 8.0 Hz, 1H), 7.66 (dd, J=4.8, 8.4 Hz, 1H). ¹³C NMR (DMSO-d6, 400 MHz): 162.9, 161.3, 152.5, 139.4, 134.4, 129.7, 121.6, 116.6.

HRMS (ES-MS) [(M+H)⁺]: for C₉H₅N₆OBr 292.9781, found 292.9783.

### Example 15

### 4-(3H-[1,2,3]Triazolo[4,5-b]pyridin-3-yloxy)-1-methylpyrimidin-2(1H)-one

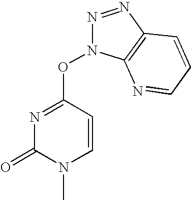

This compound was synthesized according to Example 11 from 1-methylpyrimidine-2,4(1H,3H)-dione (126 mg, 1.00 mmol), PyAOP (572 mg, 1.10 mmol) and DBU (0.17 mL, 1.20 mmol) in MeCN (10 mL) and was purified by flash chromatography as a white solid (220 mg, 90%).

¹H NMR (DMSO-d6, 400 MHz): δ (ppm) 8.83 (dd, J=1.2, 4.8 Hz, 1H), 8.75 (dd, J=1.6, 8.4 Hz, 1H), 8.47 (d, J=6.8 Hz, 1H), 7.66 (dd, J=4.4, 8.4 Hz, 1H), 6.71 (d, J=7.2 Hz, 1H). ¹³C NMR (DMSO-d6, 400 MHz): 169.9, 154.6, 152.9, 140.1, 134.6, 130.1, 122.0, 89.9, 38.1.

HRMS (ES-MS) [(M+H)⁺]: for C₁₀H₈N₆O₂ 245.0781, found 245.0785.

### Example 16

### 3-(6-Chloropyrimidin-4-yloxy)-3H-[1,2,3]triazolo[4,5-b]pyridine

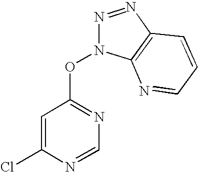

This compound was synthesized according to Example 11 from 6-chloropyrimidin-4(3H)-one (131 mg, 1.00 mmol), PyAOP (572 mg, 1.10 mmol) and DIPEA (0.19 mL, 1.23 mmol) in MeCN (10 mL) and was purified by flash chromatography as a white solid (78 mg, 60%).

¹H NMR (CDCl₃, 400 MHz): δ (ppm) 8.72 (dd, J=1.6, 4.8 Hz, 1H), 8.50-8.48 (m, 2H), 7.49 (dd, J=4.4, 8.0 Hz, 1H), 7.35 (d, J=1.2 Hz, 1H). ¹³C NMR (CDCl₃, 100 MHz): δ (ppm) 169.9, 163.1, 158.3, 152.0, 141.1, 134.9, 129.7, 121.1, 106.7.

HRMS (ES-MS) [(M+H)⁺]: for C₁₀H₆ClN₅O 249.0286, found 249.0282.

### Example 17

### 4,6-Bis(3H-[1,2,3]triazolo[4,5-b]pyridin-3-yloxy)pyrimidine

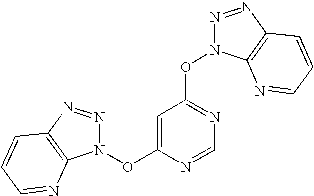

This compound was synthesized according to Example 11 from 6-chloropyrimidin-4(3H)-one (131 mg, 1.00 mmol), PyAOP (572 mg, 1.10 mmol) and DIPEA (0.19 mL, 1.23 mmol) in MeCN (10 mL) and was purified by flash chromatography as a white solid (89 mg, 26%).

¹H NMR (CDCl₃, 400 MHz): δ (ppm) 8.73 (dd, J=1.2, 4.4 Hz, 2H), 8.48 (dd, J=1.2, 8.4 Hz, 2H), 8.17 (d, J=0.8 Hz, 1H), 7.48 (dd, J=4.0, 8.0 Hz, 1H), 7.15 (d, J=0.8 Hz, 1H). ¹³C NMR (CDCl₃, 100 MHz): δ (ppm) 171.2, 158.1, 152.0, 134.9, 129.7, 121.1, 89.5.

HRMS (ES-MS) [(M+H)⁺]: for C₁₄H₁₈N₁₀O₂ 349.2, found 349.2.

### Example 18

### 4-Phenoxy-quinazoline (According to Scheme 4)

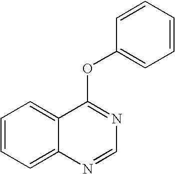

To a solution of 4-(3H-[1,2,3]triazolo[4,5-b]pyridin-3-yloxy)quinazoline (Example 11) (50 mg, 0.19 mmol), phenyl boronic acid (54 mg, 0.42 mmol) and Cs₂CO₃ (0.76 mmol, 247 mg) in DME (2 mL) was added Pd(PPh₃)₄ (32 mg). Then the reaction mixture was stirred under oxygen atmosphere for 10 h at 45° C. Then solvent was removed by rotavap and residue was purified by silica gel chromatography (20% EtOAc/Hex) to give desired product (34 mg, 80%).

¹H NMR (DMSO-d6, 400 MHz): δ (ppm) 8.85 (s, 1H), 8.26 (d, J=8.2 Hz, 1H), 8.05-8.00 (m, 2H), 7.80 (t, J=6.3 Hz, 1H), 7.68-7.66 (m, 2H), 7.56-7.54 (m, 3H). ¹³C NMR (DMSO-d6, 400 MHz): 170.6, 153.8, 148.1, 136.1, 134.9, 130.2, 129.9, 128.8, 128.7, 126.8, 123.9, 122.7.

HRMS (ES-MS) [(M+H)⁺]: for C₁₄H₁₀N₂O₁ 223.0861, found 223.0862.

### Example 19

### 4-(3-Nitrophenoxy)quinazoline

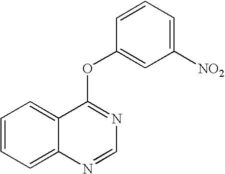

This compound was synthesized according to Example 18 from 4-(3H-[1,2,3]triazolo[4,5-b]pyridin-3-yloxy)quinazoline (73 mg, 0.28 mmol), 3-nitrophenylboronic acid (103 mg, 0.62 mmol), Cs₂CO₃ (367 mg, 1.12 mmol) and Pd(PPh₃)₄ (48 mg) in DME (3 mL). Purified by flash chromatography as a white solid (57 mg, 77%).

¹H NMR (CDCl₃, 400 MHz): δ (ppm) 8.77 (s, 1H), 8.39-8.37 (m, 1H), 8.22-8.20 (m, 2H), 8.05 (d, J=8.4 Hz, 1H), 7.98-7.95 (m, 1H), 7.74-7.66 (m, 2H). ¹³C NMR (CDCl₃, 100 MHz): δ (ppm) 166.2, 153.7, 152.6, 151.9, 149.0, 130.2, 134.5, 128.4, 128.1, 128.0, 123.2, 120.9, 117.8, 116.0. 289.

HRMS (ES-MS) [(M+H)⁺]: for C₁₄H₉N₃O₃ 268.0176, found 268.0174.

### Example 20

### 4-(4-Methoxyphenoxy)quinazoline

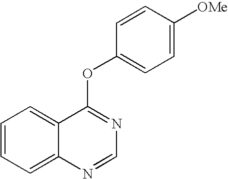

This compound was synthesized according to Example 18 from 4-(3H-[1,2,3]triazolo[4,5-b]pyridin-3-yloxy)quinazoline (73 mg, 0.28 mmol, 3-methoxyphenylboronic acid (95 mg, 0.62 mmol), Cs₂CO₃ (367 mg, 1.12 mmol) and Pd(PPh₃)₄ (48 mg) in DME (3 mL). Purified by flash chromatography as a white solid (55 mg, 78%).

¹H NMR (CDCl₃, 400 MHz): δ (ppm) 8.77 (s, 1H), 8.39-8.73 (m, 1H), 8.01 (d, J=2.5 Hz, 1H), 7.94-7.90 (m, 1H), 7.68-7.64 (m, 1H), 7.20-7.17 (m, 2H), 7.01-6.99 (m, 2H). ¹³C NMR (CDCl₃, 100 MHz): δ (ppm) 167.2, 157.3, 154.3, 151.5, 145.6, 134.0, 127.8, 127.5, 123.6, 122.6, 116.4, 114.8, 55.6.

HRMS (ES-MS) [(M+H)⁺]: for C₁₅H₁₂N₂O₂ 253.0971, found 253.0964.

### Example 21

### 4-(4-(Trifluoromethyl)phenoxy)quinazoline

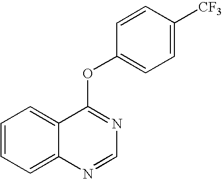

This compound was synthesized according to Example 18 from 4-(3H-[1,2,3]triazolo[4,5-b]pyridin-3-yloxy)quinazoline (100 mg, 0.38 mmol) 4-(trifluoromethyl)phenylboronic acid (159 mg, 0.83 mmol), Cs₂CO₃ (495 mg, 1.52 mmol) and Pd(PPh₃)₄ (43 mg) in DME (4 mL) and was purified by flash chromatography as a white solid (80 mg, 73%).

¹H NMR (DMSO-d6, 400 MHz): δ (ppm) 8.76 (s, 1H), 8.40 (dd, J=0.4, 8.4 Hz, 1H), 8.10-8.02 (m, 2H), 7.90 (d, J=8.8 Hz, 2H), 7.84-7.80 (m, 1H), 7.64 (d, J=8.8 Hz, 2H). ¹³C NMR (DMSO-d6, 400 MHz): 166.3, 155.2, 153.9, 151.5, 135.1, 128.5, 127.9, 127.5, 127.4, 123.7, 123.5, 115.8.

HRMS (ES-MS) [(M+H)⁺]: for C₁₅H₉N₂OF₃ 231.0739, found 291.0745.

### Example 22

### 1-(4-(Quinazolin-4-yloxy)phenyl)ethanone

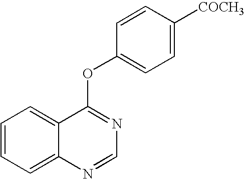

This compound was synthesized according to Example 18 from 4-(3H-[1,2,3]triazolo[4,5-b]pyridin-3-yloxy)quinazoline (350 mg, 0.38 mmol), 4-acetylphenylboronic acid (472 mg, 2.90 mmol), Cs₂CO₃ (1.79 g, 5.32 mmol) and Pd(PPh₃)₄ (231 mg) in DME (10 mL) and was purified by flash chromatography as a white solid (237 mg, 68%).

¹H NMR (CDCl₃, 400 MHz) δ (ppm) 8.78 (s, 1H), 8.38 (d, J=8.0 Hz, 1H), 8.11 (d, J=8.8 Hz, 2H), 8.50-8.31 (m, 1H), 7.95 (t, J=5.0 Hz, 1H), 7.70 (t, J=5.0 Hz, 1H), 7.38 (d, J=8.8 Hz, 2H), 2.64 (s, 3H). ¹³C NMR (CDCl₃, 100 MHz): δ (ppm) 197.1, 166.8, 156.5, 154.3, 152.1, 132.2, 134.7, 130.6, 128.3, 128.2, 123.8, 122.74, 116.6, 27.0.

HRMS (ES-MS) [(M+H)⁺]: for C₁₆H₂N₂O₂ 265.0971, found 265.0968.

### Example 23

### 4-(p-Tolyloxy)quinazoline

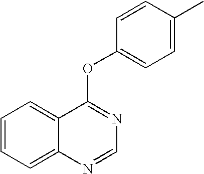

This compound was synthesized according to Example 18 from 4-(3H-[1,2,3]triazolo[4,5-b]pyridin-3-yloxy)quinazoline (100 mg, 0.38 mmol), p-tolylboronic acid (113 mg, 0.83 mmol), Cs₂CO₃ (495 mg, 1.52 mmol) and Pd(PPh₃)₄ (43 mg) in DME (4 mL) and was purified by flash chromatography as a white solid (61 mg, 69

¹H NMR (CDCl₃, 400 MHz) δ (ppm) 8.77 (s, 1H), 8.39-8.36 (m, 1H), 8.00 (d, J=8.4 Hz, 1H), 7.92-7.85 (m, 2H), 7.70-7.61 (m, 1H), 7.28 (d, J=8.0 Hz, 2H), 7.15-7.13 (m, 3H), 2.40 (s, 3H). ¹³C NMR (CDCl₃, 100 MHz): 167.5, 154.7, 151.9, 150.4, 136.1, 134.4, 130.7, 128.2, 127.8, 124.0, 121.9, 116.8, 21.3.

HRMS (ES-MS) [(M+H)⁺]: for C₁₅H₁₂N₂O 237.1022, found 237.1022.

### Example 24

### 4-(Quinazolin-4-yloxy)benzonitrile

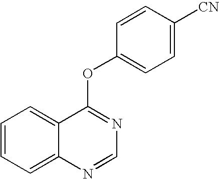

This compound was synthesized according to Example 18 from 4-(3H-[1,2,3]triazolo[4,5-b]pyridin-3-yloxy)quinazoline (73 mg, 0.28 mmol), 4-cyanophenylboronic acid (91 mg, 0.83 mmol), Cs₂CO₃ (364 mg, 1.12 mmol) and Pd(PPh₃)₄ (48 mg) in DME (3 mL) and was purified by flash chromatography as a white solid (53 mg, 77%).

¹H NMR (CDCl₃, 400 MHz): δ (ppm) 8.77 (s, 1H), 8.37-8.35 (m, 1H), 8.05 (d, J=8.4 Hz, 1H), 7.98-7.94 (m, 1H), 7.82-7.78 (m, 2H), 7.73-7.69 (m, 2H), 7.45-7.42 (m 2H). ¹³C NMR (CDCl₃, 100 MHz): 166.1, 155.7, 153.7, 151.9, 134.5, 133.9, 128.1, 128.0, 123.3, 118.2, 116.0, 109.9.

HRMS (ES-MS) [(M+H)⁺]: for C₁₅H₉N₃O 248.0818, found 248.0811.

### Example 25

### 4-(6-Chloropyridin-3-yloxy)quinazoline

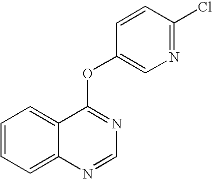

This compound was synthesized according to Example 18 from 4-(3H-[1,2,3]triazolo[4,5-b]pyridin-3-yloxy)quinazoline (73 mg, 0.28 mmol), 6-chloropyridin-3-ylboronic acid (97 mg, 0.62 mmol), Cs₂CO₃ (364 mg, 1.12 mmol) and Pd(PPh₃)₄ (48 mg) in DME (3 mL) and was purified by flash chromatography as a white solid (52 mg, 72%).

¹H NMR (CDCl₃, 400 MHz) δ (ppm) 8.76 (s, 1H), 8.42 (d, J=0.40 Hz, 1H), 8.37-8.35 (m, 1H), 8.05 (d, J=8.4 Hz, 1H), 7.98-7.94 (m, 1H), 7.73-7.66 (m, 2H), 7.46 (dd, J=0.3, 8.4 Hz, 1H). ¹³C NMR (CDCl₃, 100 MHz): 166.0, 153.6, 151.9, 148.1, 147.9, 143.5, 134.5, 132.7, 128.1, 128.0, 124.8, 123.2, 115.9.

HRMS (ES-MS) [(M+H)⁺]: for C₁₃H₈N₃OCl 258.0428, found 258.0422.

### Example 26

### 3,5-Dimethyl-4-(quinazolin-4-yloxy)isoxazole

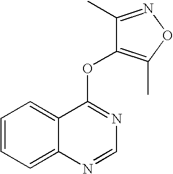

This compound was synthesized according to Example 18 from 4-(3H-[1,2,3]triazolo[4,5-b]pyridin-3-yloxy)quinazoline (50 mg, 0.19 mmol), 3,5-dimethylisoxazol-4-ylboronic acid (53 mg, 0.38 mmol), Cs₂CO₃ (247 mg, 0.76 mmol) and Pd(PPh₃)₄ (33 mg) in DME (3 mL) and was purified by flash chromatography as a white solid (30 mg, 60%).

¹H NMR (DMSO-d6, 400 MHz): δ (ppm) 8.81 (s, 1H), 8.41-8.38 (m, 1H), 8.01-8.03 (m, 2H), 7.85-7.81 (d, 2H), 2.34 (s, 3H), 2.12 (s, 3H). ¹³C NMR (DMSO-d6, 400 MHz): 164.7, 158.7, 154.9, 153.5, 151.1, 134.8, 129.3, 128.3, 127.4, 123.2, 114.8, 10.3, 9.0.

HRMS (ES-MS) [(M+H)⁺]: for C₁₃H₁₁N₃O₂ 242.0924, found 242.0928.

### Example 27

### 4-(1-Benzyl-1H-pyrazol-3-yloxy)quinazoline

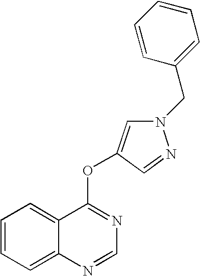

This compound was synthesized according to Example 18 from 4-(3H-[1,2,3]triazolo[4,5-b]pyridin-3-yloxy)quinazoline (50 mg, 0.19 mmol), 3,5-dimethylisoxazol-4-ylboronic acid (76 mg, 0.38 mmol), Cs₂CO₃ (247 mg, 0.76 mmol) and Pd(PPh₃)₄ (33 mg) in DME (3 mL) and was purified by flash chromatography as a yellow solid (35 mg, 62%).

HRMS (ES-MS) [(M+H)⁺]: for C₁₈H₁₄N₄O 303.1240, found 303.1245.

### Example 28

### 4-(2-Methoxypyridin-3-yloxy)quinazoline

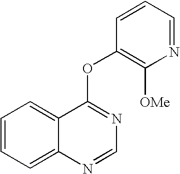

This compound was synthesized according to Example 18 from 4-(3H-[1,2,3]triazolo[4,5-b]pyridin-3-yloxy)quinazoline (50 mg, 0.19 mmol), 2-methoxypyridin-3-ylboronic acid (109 mg, 0.41 mmol), Cs₂CO₃ (247 mg, 0.76 mmol) and Pd(PPh₃)₄ (33 mg) in DME (3 mL) and was purified by flash chromatography as a white solid (40 mg, 83%).

¹H NMR (CDCl₃, 400 MHz) δ (ppm) 8.74 (s, 1H), 8.39 (dd, J=0.8, 4.8 Hz, 1H), 8.13 (dd, J=1.6, 4.8 Hz, 1H), 8.02 (d, J=8.4 Hz, 1H), 7.94-7.92 (m, 1H), 7.72-7.65 (m, 1H), 7.53 (dd, J=1.6, 7.6 Hz, 1H), 7.01 (dd, J=5.2, 7.6 Hz, 1H). ¹³C NMR (CDCl₃, 100 MHz): 166.2, 155.6, 154.0, 151.7, 143.9, 136.3, 1341, 130.7, 127.8, 127.6, 123.7, 117.0, 116.0, 53.6.

HRMS (ES-MS) [(M+H)⁺]: for C₁₄H₁₁N₃O₂ 254.0924, found 254.0927.

### Example 29

### 4-(1H-Indol-5-yloxy)quinazoline

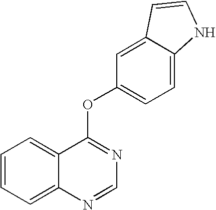

This compound was synthesized according to Example 18 from 4-(3H-[1,2,3]triazolo[4,5-b]pyridin-3-yloxy)quinazoline (73 mg, 0.28 mmol), 1H-indol-5-ylboronic acid (100 mg, 0.62 mmol), Cs₂CO₃ (364 mg, 1.12 mmol) and Pd(PPh₃)₄ (48 mg) in DME (3 mL) and was purified by flash chromatography as a white solid (28 mg, 39%).

¹H NMR (CDCl₃, 400 MHz) δ (ppm) 8.77 (s, 1H), 8.46-8.44 (m, 1H), 8.33 (s, 1H), 8.01 (d, J=8.0 Hz, 1H), 7.94-7.89 (m, 1H), 7.50 (d, J=2.4 Hz, 1H), 7.69-7.65 (m, 1H), 7.50 (d, J=2.4 Hz, 1H), 7.46 (d, J=8.4 Hz, 1H), 7.27 (t, J=3.2 Hz, 1H), 7.08 (dd, J=2.0, 8.4 Hz, 1H) 6.59-6.58 (m, 1H)). ¹³C NMR (CDCl₃, 100 MHz): 167.8, 154.5, 151.5, 146.0, 133.9, 133.8, 128.4, 127.8, 127.4, 125.6, 123.8, 116.6, 116.2, 112.8, 111.7, 103.2.

HRMS (ES-MS) [(M+H)⁺]: for C₁₆H₁₁N₃O 272.0974 found 272.0966.

### Example 30

### 4-(Thiophen-3-yloxy)quinazoline

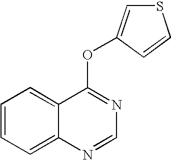

This compound was synthesized according to Example 18 from 4-(3H-[1,2,3]triazolo[4,5-b]pyridin-3-yloxy)quinazoline (73 mg, 0.28 mmol), thiophen-3-ylboronic acid (100 mg, 0.83 mmol), Cs₂CO₃ (495 mg, 1.52 mmol) and Pd(PPh₃)₄ (43 mg) in DME (3 mL) and was purified by flash chromatography as a brown solid (30 mg, 34%).

HRMS (ES-MS) [(M+H)⁺]: for C₁₂H₈N₂OS 229.0432 found 229.0430.

### Example 31

### 4-(Pyrimidin-5-yloxy)quinazoline

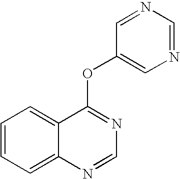

This compound was synthesized according to Example 18 from 4-(3H-[1,2,3]triazolo[4,5-b]pyridin-3-yloxy)quinazoline (50 mg, 0.19 mmol, pyrimidin-5-ylboronic acid (51 mg, 0.41 mmol), Cs₂CO₃ (247 mg, 0.76 mmol) and Pd(PPh₃)₄ (33 mg) in DME (3 mL) and was purified by flash chromatography as a white solid (32 mg, 76%).

¹H NMR (CDCl₃, 400 MHz): δ (ppm) 9.06 (s, 1H), 8.41 (s, 2H), 8.77 (s, 1H), 8.39-8.37 (m, 1H), 8.06-7.96 (m, 2H), 7.76-7.73 (m, 1H). ¹³C NMR (CDCl₃, 100 MHz): δ (ppm) 166.0, 156.0, 153.8, 152.4, 151.2, 147.9, 135.0, 128.6, 128.5, 123.5, 116.1

MS (ES-MS) [(M+H)⁺]: for C₁₂H₈N₄O₂ 225.2, found 225.2

### Example 32

### 3-(Thieno[2,3-d]pyrimidin-4-yloxy)benzonitrile

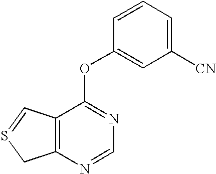

This compound was synthesized according to Example 18 from 4-(3H-[1,2,3]triazolo[4,5-b]pyridin-3-yloxy)thieno[2,3-d]pyrimidine (68 mg, 0.25 mmol), 3-cyanophenylboronic acid (80 mg, 0.55 mmol), Cs₂CO₃ (325 mg, 1.00 mmol) and Pd(PPh₃)₄ (43 mg) in DME (3 mL). It was purified by flash chromatography as a white solid (56 mg, 88%).

¹H NMR (CDCl₃, 400 MHz) δ (ppm) 8.73 (s, 1H), 8.01 (d, J=5.2 Hz, 1H), 7.63-7.69 (m, 4H). ¹³C NMR (CDCl₃, 100 MHz): 163.6, 163.2, 154.0, 152.0, 135.6, 130.6, 129.7, 126.8, 125.6, 124.8, 117.7, 113.7.

HRMS (ES-MS) [(M+H)⁺]: for C₁₃H₇N₃OS 254.0383, found 254.0382.

### Example 33

### 4-Phenoxythieno[2,3-d]pyrimidine

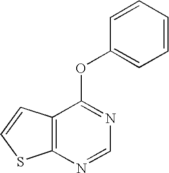

This compound was synthesized according to Example 18 from 4-(3H-[1,2,3]triazolo[4,5-b]pyridin-3-yloxy)thieno[2,3-d]pyrimidine (68 mg, 0.25 mmol), phenylboronic acid (66 mg, 0.55 mmol), Cs₂CO₃ (325 mg, 1.00 mmol) and Pd(PPh₃)₄ (43 mg) in DME (3 mL). It was purified by flash chromatography as a white solid (50 mg, 87%).

¹H NMR (CDCl₃, 400 MHz) δ (ppm) 8.73 (s, 1H), 7.96 (d, J=5.6 Hz, 1H), 7.56 (d, J=5.6 Hz, 1H), 7.50-7.45 (m, 2H) 7.34-7.26 (m, 3H). ¹³C NMR (CDCl₃, 100 MHz): 164.5, 163.5, 154.8, 152.3, 135.4, 130.1, 126.5, 125.0, 122.2, 118.2.

HRMS (ES-MS) [(M+H)⁺]: for C₁₃H₇N₃O₃S 274.0281, found 274.0281.

### Example 34

### 4-(3-Nitrophenoxy)thieno[2,3-d]pyrimidine

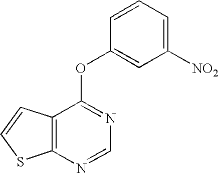

This compound was synthesized according to Example 18 from 4-(3H-[1,2,3]triazolo[4,5-b]pyridin-3-yloxy)thieno[2,3-d]pyrimidine (68 mg, 0.25 mmol), 3-nitrophenylboronic acid (91 mg, 0.55 mmol), Cs₂CO₃ (325 mg, 1.00 mmol) and Pd(PPh₃)₄ (43 mg) in DME (3 mL). It was purified by flash chromatography as a white solid (53 mg, 78%).

¹H NMR (CDCl₃, 400 MHz) δ (ppm) 8.73 (s, 1H), 8.21-8.20 (m, 1H), 8.03 (d, J=5.2 Hz, 1H), 7.67-7.65 (m, 2H), 7.61 (d, J=5.2 Hz, 1H). ¹³C NMR (CDCl₃, 100 MHz): 16.36, 163.3, 154.0, 152.2, 135.6, 130.2, 128.3, 124.8, 120.9, 117.6.

HRMS (ES-MS) [(M+H)⁺]: for C₁₂H₈N₂OS 229.0430, found 229.0430.

### Example 35

### 3,5-Dimethyl-4-(thieno[2,3-d]pyrimidin-4-yloxy)isoxazole

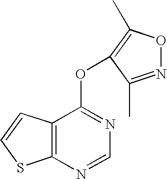

This compound was synthesized according to Example 18 from 4-(3H-[1,2,3]triazolo[4,5-b]pyridin-3-yloxy)thieno[2,3-d]pyrimidine (80 mg, 0.29 mmol), 3,5-dimethylisoxazol-4-ylboronic acid (91 mg, 0.65 mmol), Cs₂CO₃ (337 mg, 1.16 mmol) and Pd(PPh₃)₄ (50 mg) in DME (3 mL). It was purified by flash chromatography as a white solid (51 mg, 71%).

¹H NMR (DMSO-d6, 400 MHz): δ (ppm) 8.78 (s, 1H), 8.52 (d, J=5.2 Hz, 1H), 7.72 (d, J=5.2 Hz, 1H), 2.32 (s, 3H), 2.10 (s, 1H). ¹³C NMR (DMSO-d6, 400 MHz): 163.3, 162.1, 158.9, 154.9, 153.9, 137.8, 129.1, 124.2, 116.2, 9.9, 8.9.

MS (ES-MS) [(M+H)⁺]: for C₁₁H₉N₃O₂S 248.1, found 248.1.

### Example 36

### Methyl 5-methyl-4-phenoxythieno[2,3-d]pyrimidine-6-carboxylate

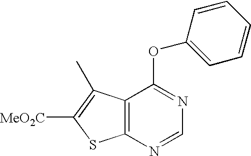

This compound was synthesized according to Example 18 methyl 4-(3H-[1,2,3]triazolo[4,5-b]pyridin-3-yloxy)-5-methylthieno[2,3-d]pyrimidine-6-carboxylate (85 mg, 0.25 mmol), phenylboronic acid (66 mg, 0.55 mmol), Cs₂CO₃ (325 mg, 1.00 mmol) and Pd(PPh₃)₄ (43 mg) in DME (3 mL). Purified by flash chromatography as a white solid (60 mg, 80%).

¹H NMR (CDCl₃, 400 MHz) δ (ppm) 8.62 (s, 1H), 7.51-7.47 (m, 2H), 7.35-7.31 (m, 1H), 7.26-7.22 (m, 2H), 3.96 (s, 3H), 3.05 (s, 3H). ¹³C NMR (CDCl₃, 100 MHz): 169.2, 165.6, 162.9, 155.2, 151.8, 140.0, 129.8, 126.1, 125.0, 121.8, 120.5, 52.4, 15.6.

HRMS (ES-MS) [(M+H)⁺]: for C₁₅H₁₂N₂O₃S 301.0641, found 301.0641.

### Example 37

### Methyl 5-methyl-4-(3-nitrophenoxy)thieno[2,3-d]pyrimidine-6-carboxylate

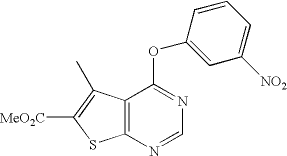

This compound was synthesized according to Example 18 from methyl 4-(3H-[1,2,3]triazolo[4,5-b]pyridin-3-yloxy)-5-methylthieno[2,3-d]pyrimidine-6-carboxylate (85 mg, 0.25 mmol), 3-nitrophenylboronic acid (91 mg, 0.55 mmol), Cs₂CO₃ (325 mg, 1.00 mmol) and Pd(PPh₃)₄ (43 mg) in DME (3 mL). It was purified by flash chromatography as a white solid (64 mg, 76%).

¹H NMR (CDCl₃, 400 MHz) δ (ppm) 8.62 (s, 1H) 8.20-8.16 (m, 2H), 7.69-7.62 (m, 2H), 3.97 (s, 3H), 3.05 (s, 3H). ¹³C NMR (CDCl₃, 100 MHz): 164.7, 162.8, 154.7, 152.1, 139.8, 130.3, 128.4, 125.9, 121.0, 119.8, 117.8, 52.5, 15.6.

HRMS (ES-MS) [(M+H)⁺]: for C₁₅H₁₁N₂O₅S 346.0492, found 346.0490.

### Example 38

### Methyl 4-(3-cyanophenoxy)-5-methylthieno[2,3-d]pyrimidine-6-carboxylate

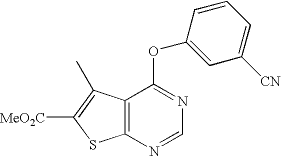

This compound was synthesized according to Example 18 from methyl 4-(3H-[1,2,3]triazolo[4,5-b]pyridin-3-yloxy)-5-methylthieno[2,3-d]pyrimidine-6-carboxylate (85 mg, 0.25 mmol), cyanophenylboronic acid (80 mg, 0.55 mmol), Cs₂CO₃ (325 mg, 1.00 mmol) and Pd(PPh₃)₄ (43 mg) in DME (3 mL). It was purified by flash chromatography as a white solid (59 mg, 73%).

¹H NMR (CDCl₃, 400 MHz) δ (ppm) 8.62 (s, 1H), 7.63-7.53 (m, 5H), 3.97 (s, 3H), 3.0 (s, 3H). ¹³C NMR (CDCl₃, 100 MHz): 169.6, 164.7, 162.8, 154.7, 151.9, 139.4, 130.7, 130.5, 129.8, 126.9, 125.8, 119.8, 117.7, 113.8, 52.5, 15.6.

MS (ES-MS) [(M+H)⁺]: for C₁₆H₁₁N₃O₃S 326.2, found 326.2.

### Example 39

### Methyl 4-(3,5-dimethyl isoxazol-4-yloxy)-5-methylthieno[2,3-d]pyrimidine-6-carboxylate

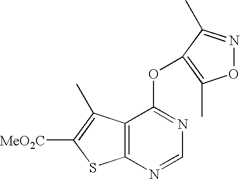

This compound was synthesized according to Example 18 from methyl 4-(3H-[1,2,3]triazolo[4,5-b]pyridin-3-yloxy)-5-methylthieno[2,3-d]pyrimidine-6-carboxylate (85 mg, 0.25 mmol), 3,5-dimethylisoxazol-4-ylboronic acid (77 mg, 0.55 mmol), Cs₂CO₃ (325 mg, 1.00 mmol) and Pd(PPh₃)₄ (43 mg) in DME (3 mL). Purified by flash chromatography as a white solid (62 mg, 78%).

¹H NMR (CDCl₃, 400 MHz) δ (ppm) 8.64 (s, 1H), 3.96 (s, 3H), 3.02 (s, 3H), 2.35 (s, 3H), 2.19 (s, 3H). ¹³C NMR (CDCl₃, 100 MHz): 169.4, 164.1, 158.7, 155.0, 154.9, 139.2, 118.4, 52.5, 29.7, 15.7, 10.7, 9.78.

HRMS (ES-MS) [(M+H)⁺]: for C₁₄H₁₃N₃O₄S 320.0700, found 320.0701.

### Example 40

### 5-Bromo-2-phenoxypyrimidine

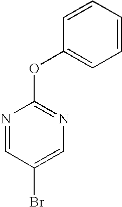

3-(5-Bromo-pyrimidin-2-yloxy)-3H-[1,2,3]triazolo[4,5-b]pyridine (100 mg, 0.34 mmole) was dissolved in DME (4 mL) at RT and phenyl boronic acid (125 mg, 1.02 mmole) was added to it. Cs₂CO₃ (443 mg, 1.36 mmole) and Pd(PPh₃)₄ (39 mg, 0.03 mmole) was added to the reaction mixture and purged with O₂. The reaction mixture was then stirred at RT for 10 h and was directly purified by flash chromatography to afford a white solid (60 mg, 70%).

¹H-NMR (CDCl₃, 300 MHz) δ (ppm) 8.57 (s, 2H), 7.44 (m, 2H), 7.26 (m, 1H), 7.17 (d, 2H, J=4.2 Hz). LCMS (ES-MS) [(M+H)+]: for C₁₀H₇BrN₂O 251.07, found 251.30.

### Example 41

### 5-Bromo-2-(4-iodo-phenoxy)-pyrimidine

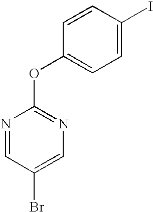

This compound was synthesized according to Example 40 from 3-(5-bromo-pyrimidin-2-yloxy)-3H-[1,2,3]triazolo[4,5-b]pyridine (100 mg, 0.34 mmole), 4-iodo phenyl boronic acid (254 mg, 1.02 mmole), Cs₂CO₃ (443 mg, 1.36 mmole), and Pd(PPh₃)₄ (39 mg, 0.03 mmole) in DME and was purified by flash chromatography as a white solid (41 mg, 32%).

¹H-NMR (CDCl₃, 300 MHz) δ (ppm) 8.57 (s, 2H), 7.75 (d, 2H, J=9 Hz), 6.97 (d, 2H, J=8.4 Hz).

LCMS (ES-MS) [(M+H)+]: for C₁₀H₆BrIN₂O 376.97, found 377.20.

### Example 42

### 1-[4-(5-Bromo-pyrimidin-2-yloxy)-phenyl]-ethanone

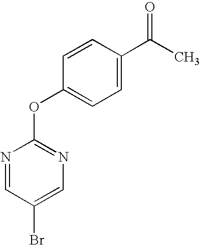

This compound was synthesized according to Example 40 from 3-(5-bromo-pyrimidin-2-yloxy)-3H-[1,2,3]triazolo[4,5-b]pyridine (20 mg, 0.07 mmole), 4-acetyl phenyl boronic acid (24 mg, 0.15 mmole), Cs₂CO₃ (88 mg, 0.27 mmole), and Pd(PPh₃)₄ (8 mg, 0.01 mmole) in DME and was purified by flash chromatography as a white solid (19 mg, 95%).

¹H-NMR (CDCl₃, 300 MHz) δ (ppm) 8.87 (s, 2H), 8.06 (d, 2H, J=8.7 Hz), 7.40 (d, 2H, J=8.7 Hz), 3.33 (s, 3H).

LCMS (ES-MS) [(M+H)+]: for C₁₂H₉BrN₂O₂ 293.11, found 293.30.

### Example 43

### 1-[3-(5-Bromo-pyrimidin-2-yloxy)-phenyl]-ethanone

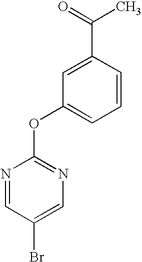

This compound was synthesized according to Example 40 from 3-(5-bromo-pyrimidin-2-yloxy)-3H-[1,2,3]triazolo[4,5-b]pyridine (50 mg, 0.17 mmole), 3-acetyl phenyl boronic acid (84 mg, 0.51 mmole), Cs₂CO₃ (222 mg, 0.68 mmole), and Pd(PPh₃)₄ (20 mg, 0.01 mmole) in DME (2.5 mL) and was purified by flash chromatography as a white solid (50 mg, 100%).

¹H-NMR (CDCl₃, 300 MHz) δ (ppm) 8.58 (s, 2H), 7.88 (m, 1H), 7.78 (t, 1H, J=2.1 Hz), 7.55 (t, 1H, J=7.8 Hz), 7.42 (m, 1H), 2.57 (s, 3H).

LCMS (ES-MS) [(M+H)+]: for C₁₂H₉BrN₂O₂ 293.11, found 293.30.

### Example 44

### 1-[2-(5-Bromo-pyrimidin-2-yloxy)-phenyl]-ethanone

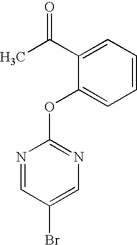

This compound was synthesized according to Example 40 from 3-(5-bromo-pyrimidin-2-yloxy)-3H-[1,2,3]triazolo[4,5-b]pyridine (20 mg, 0.07 mmole), 2-acetyl phenyl boronic acid (25 mg, 0.15 mmole), Cs₂CO₃ (88 mg, 0.27 mmole), and Pd(PPh₃)₄ (8 mg, 0.27 mmole) in DME (1 mL) and was purified by flash chromatography as a white solid (7 mg, 34%).

¹H-NMR (CDCl₃, 300 MHz) δ (ppm) 8.56 (s, 2H), 7.90 (dd, 1H, J=1.8 Hz, J=7.8 Hz), 7.60 (td, 1H, J=1.8 Hz, J=7.5 Hz), 7.38 (td, 1H, J=0.9 Hz, J=7.8 Hz), 7.23 (dd, 1H, J=0.9 Hz, J=7.5 Hz), 2.52 (s, 3H).

HRMS (ES-MS) [(M+H)+]: for C₁₂H₉BrN₂O₂ 292.9920, found 292.9919.

### Example 45

### 5-Bromo-2-(4-vinyl-phenoxy)-pyrimidine

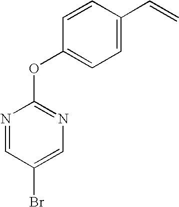

This compound was synthesized according to Example 40 from 3-(5-bromo-pyrimidin-2-yloxy)-3H-[1,2,3]triazolo[4,5-b]pyridine (100 mg, 0.34 mmole), 4-vinyl phenyl boronic acid (152 mg, 1.02 mmole), Cs₂CO₃ (443 mg, 1.36 mmole), and Pd(PPh₃)₄ (39 mg, 0.03 mmole) in DME (10 mL) and was purified by flash chromatography as a white solid (45 mg, 48%).

¹H-NMR (CDCl₃, 300 MHz) δ (ppm) 8.57 (s, 2H), 7.48 (d, 2H, J=6.3 Hz), 7.16 (d, 2H, J=6.9 Hz), 6.78 (dd, 1H, J=10.8 Hz, J=6.9 Hz), 5.75 (d, 1H, J=17.1 Hz), 5.28 (d, 1H, J=10.8 Hz).

LCMS (ES-MS) [(M)+]: for C₁₂H₉BrN₂O 277.11, found 277.30.

### Example 46

### 4-(5-Bromo-pyrimidin-2-yloxy)-benzoic Acid Methyl Ester

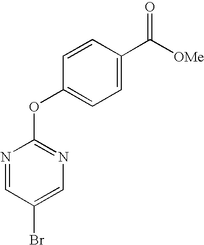

This compound was synthesized according to Example 40 from 3-(5-bromo-pyrimidin-2-yloxy)-3H-[1,2,3]triazolo[4,5-b]pyridine (20 mg, 0.07 mmole), 4-(carboxymethyl)-phenyl boronic acid (27 mg, 0.15 mmole), Cs₂CO₃ (88 mg, 0.27 mmole), and Pd(PPh₃)₄ (8 mg, 0.27 mmole) in DME (1 mL) and was purified by flash chromatography as a white solid (6 mg, 29%).

¹H-NMR (CDCl₃, 300 MHz) δ (ppm) 8.59 (s, 2H), 8.14 (d, 2H, J=6.6 Hz), 7.24 (d, 2H, J=6.6 Hz), 3.93 (s, 3H).

LCMS (ES-MS) [(M+H)+]: for C₁₂H₉BrN₂O₃ 309.11, found 309.30.

### Example 47

### 3-(5-Bromo-pyrimidin-2-yloxy)-benzoic Acid Methyl Ester

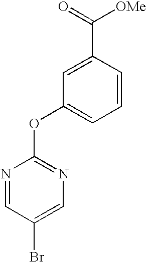

This compound was synthesized according to Example 40 from 3-(5-bromo-pyrimidin-2-yloxy)-3H-[1,2,3]triazolo[4,5-b]pyridine (50 mg, 0.17 mmole), 3-(carboxymethyl)-phenyl boronic acid (68 mg, 0.38 mmole), Cs₂CO₃ (222 mg, 0.68 mmole), and Pd(PPh₃)₄ (20 mg, 0.02 mmole) in DME (2.5 mL) and was purified by flash chromatography as a white solid (24 mg, 44%).

¹H-NMR (CDCl₃, 300 MHz) δ (ppm) 8.58 (s, 2H), 7.97 (m, 1H), 7.78 (t, 1H, J=2.4 Hz), 7.52 (t, 1H, J=7.8 Hz), 7.39 (m, 1H), 3.92 (s, 3H).

LCMS (ES-MS) [(M+Na)+]: for C₁₂H₉BrN₂O₃ 332.10, found 333.10.

### Example 48

### 2-(5-Bromo-pyrimidin-2-yloxy)-benzoic Acid Methyl Ester

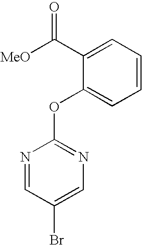

This compound was synthesized according to Example 40 from 3-(5-bromo-pyrimidin-2-yloxy)-3H-[1,2,3]triazolo[4,5-b]pyridine (50 mg, 0.17 mmole), 2-(carboxymethyl)-phenyl boronic acid (68 mg, 0.38 mmole), Cs₂CO₃ (222 mg, 0.68 mmole), and Pd(PPh₃)₄ (20 mg, 0.02 mmole) in DME (2.5 mL) and was purified by flash chromatography as a white solid (34 mg, 63%).

¹H-NMR (CDCl₃, 300 MHz) δ (ppm) 8.56 (s, 2H), 7.89 (dd, 1H, J=1.8 Hz, J=7.8 Hz), 7.60 (dd, 1H, J=1.5 Hz, J=7.5 Hz), 7.41 (dd, 1H, J=0.9 Hz, J=7.8 Hz), 7.23 (dd, 1H, J=0.9 Hz, J=8.1 Hz), 2.52 (s, 3H).

HRMS (ES-MS) [(M+H)+]: for C₁₂H₉BrN₂O₃ 308.9869, found 308.9871.

### Example 49

### 5-Bromo-2-(4-methoxy-phenoxy)-pyrimidine

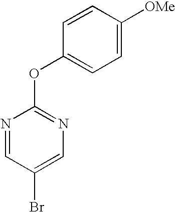

This compound was synthesized according to Example 40 from 3-(5-bromo-pyrimidin-2-yloxy)-3H-[1,2,3]triazolo[4,5-b]pyridine (20 mg, 0.07 mmole), 4-methoxy phenyl boronic acid (23 mg, 0.15 mmole), Cs₂CO₃ (88 mg, 0.27 mmole), and Pd(PPh₃)₄ (8 mg, 0.01 mmole) in DME (1 mL) and was purified by flash chromatography as a white solid (14 mg, 100%).

¹H-NMR (CDCl₃, 300 MHz) δ (ppm) 8.56 (s, 2H), 7.12 (d, 2H, J=9.3 Hz), 6.96 (d, 1H, J=9.0 Hz), 3.82 (s, 3H).

HRMS (ES-MS) [(M+H)+]: for C₁₁H₉BrN₂O₂ 280.9920, found 280.9919.

### Example 50

### 5-Bromo-2-(2-methoxy-phenoxy)-pyrimidine

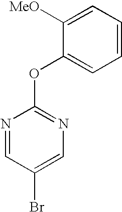

This compound was synthesized according to Example 40 from 3-(5-bromo-pyrimidin-2-yloxy)-3H-[1,2,3]triazolo[4,5-b]pyridine (20 mg, 0.07 mmole), 2-methoxy phenyl boronic acid (23 mg, 0.15 mmole), Cs₂CO₃ (88 mg, 0.27 mmole), and Pd(PPh₃)₄ (8 mg, 0.01 mmole) in DME (1 mL) and was purified by flash chromatography as a white solid (16 mg, 85%).

¹H-NMR (CDCl3, 400 MHz)

HRMS (ES-MS) [(M+H)+]: for C₁₁H₉BrN₂O₂ 280.9920, found 280.9918.

### Example 51

### 5-Bromo-2-(4-methylsulfanyl-phenoxy)-pyrimidine

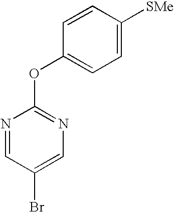

This compound was synthesized according to Example 40 from 3-(5-bromo-pyrimidin-2-yloxy)-3H-[1,2,3]triazolo[4,5-b]pyridine (20 mg, 0.07 mmole), 4-methylthio phenyl boronic acid (25 mg, 0.15 mmole), Cs₂CO₃ (88 mg, 0.27 mmole), and Pd(PPh₃)₄ (8 mg, 0.01 mmole) in DME (1 mL) and was purified by flash chromatography as a white solid (14 mg, 70%).

¹H-NMR (CDCl₃, 300 MHz) δ (ppm) 8.57 (s, 2H), 7.33 (d, 2H, J=6.6 Hz), 7.13 (d, 1H, J=6.9 Hz), 2.50 (s, 3H).

HRMS (ES-MS) [(M+H)+]: for C₁₁H₉BrN₂OS 296.9692, found 296.9688.

### Example 52

### 5-Bromo-2-(pyrimidin-5-yloxy)-pyrimidine

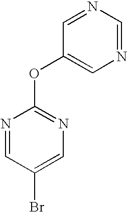

This compound was synthesized according to Example 40 from 3-(5-bromo-pyrimidin-2-yloxy)-3H-[1,2,3]triazolo[4,5-b]pyridine (20 mg, 0.07 mmole), pyrimidine-5-boronic acid (18 mg, 0.15 mmole), Cs₂CO₃ (88 mg, 0.27 mmole), and Pd(PPh₃)₄ (8 mg, 0.01 mmole) in DME (1 mL) and was purified by flash chromatography as a white solid (6 mg, 41%).

1H-NMR ((CD3)2CO, 300 MHz) δ (ppm) 8.94 (s, 1H), 8.69 (s, 2H), 8.64 (s, 2H).

LCMS (ES-MS) [(M+H)+]: for C₈H₅BrN₄O 253.05, found 253.30.

### Example 53

### 5-(5-Bromo-pyrimidin-2-yloxy)-1H-indole

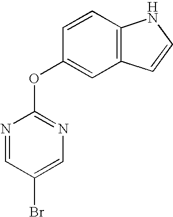

This compound was synthesized according to Example 40 from 3-(5-bromo-pyrimidin-2-yloxy)-3H-[1,2,3]triazolo[4,5-b]pyridine (20 mg, 0.07 mmole), indole-5 boronic acid (24 mg, 0.15 mmole), Cs₂CO₃ (88 mg, 0.27 mmole), and Pd(PPh₃)₄ (8 mg, 0.01 mmole) in DME (1 mL) and was purified by flash chromatography as a white solid (4 mg, 21%).

¹H-NMR (CDCl₃, 300 MHz) δ (ppm) 8.56 (s, 2H), 8.18 (s, 1H), 7.52 (m, 2H), 7.03 (m, 1H), 6.57 (m, 1H).

LCMS (ES-MS) [(M+H)+]: for C₁₂H₈BrN₃O 290.11, found 290.30.

### Example 54

### 5-Bromo-2-(furan-3-yloxy)-pyrimidine

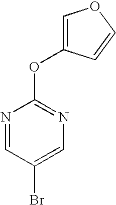

This compound was synthesized by microwave irradiation (15 mins, 90° C.) of 3-(5-bromo-pyrimidin-2-yloxy)-3H-[1,2,3]triazolo[4,5-b]pyridine (20 mg, 0.07 mmole), 3-furan boronic acid (17 mg, 0.15 mmole), Cs₂CO₃ (88 mg, 0.27 mmole), and Pd(PPh₃)₄ (8 mg, 0.01 mmole) in DME (1 mL). The crude reaction mixture was purified by flash chromatography to obtain an off-white solid (4 mg, 25%).

¹H-NMR (CDCl₃, 300 MHz) δ (ppm) 8.65 (s, 2H), 7.76 (t, 1H, J=1.2 Hz), 7.56 (t, 1H, J=2.1 Hz), 7.48 (dd, 1H, J=4.5 Hz, J=8.4 Hz).

LCMS (ES-MS) [(M+Na)+]: for C₈H₅BrN₂O₂ 264.03, found 265.20.

### Example 55

### 5-Bromo-2-(1H-pyrazol-4-yloxy)-pyrimidine

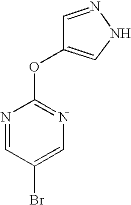

This compound was synthesized according to Example 40 from 3-(5-bromo-pyrimidin-2-yloxy)-3H-[1,2,3]triazolo[4,5-b]pyridine (20 mg, 0.07 mmole), 1H-pyrazole-5 boronic acid (17 mg, 0.15 mmole), Cs₂CO₃ (88 mg, 0.27 mmole), and Pd(PPh₃)₄ (8 mg, 0.01 mmole) in DME (1 mL) and heating to 45° C. for 10 h. The reaction mixture was purified by flash chromatography as a white solid (8 mg, 50%).

¹H-NMR (CDCl₃, 300 MHz) δ (ppm) 8.60 (s, 2H), 7.84 (d, 1H, J=1.2 Hz), 6.52 (dd, 1H, J=1.2 Hz, J=2.7 Hz).

### Example 56

### 5-Bromo-2-(pyridin-3-yl oxy)-pyrimidine

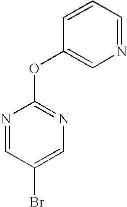

This compound was synthesized according to Example 40 from 3-(5-bromo-pyrimidin-2-yloxy)-3H-[1,2,3]triazolo[4,5-b]pyridine (40 mg, 0.14 mmole), 3-pyridine boronic acid (50 mg, 0.15 mmole), Cs₂CO₃ (177 mg, 0.54 mmole), and Pd(PPh₃)₄ (16 mg, 0.01 mmole) in DME (2 mL). The reaction mixture was purified by flash chromatography as a white solid (12 mg, 35%).

¹H-NMR (CDCl₃, 300 MHz) δ (ppm) 8.60 (s, 2H), 8.55 (m, 2H), 7.57 (qd, 1H, J=1.8 Hz, J=8.3 Hz), 7.40 (dd, 1H, J=4.8 Hz, J=8.4 Hz).

HRMS (ES-MS) [(M+H)+]: for C₉H₆BrN₃O 251.9767, found 251.9770.

### Example 57

### 5-Bromo-2-(2-methoxypyridin-3-yloxy)pyrimidine

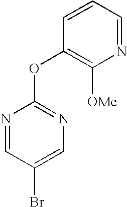

This compound was synthesized according to Example 18 from 3-(5-bromopyrimidin-2-yloxy)-3H-[1,2,3]triazolo[4,5-b]pyridine (59 mg, 0.20 mmol), methoxypyridin-3-ylboronic acid (68 mg, 0.45 mmol), Cs₂CO₃ (260 mg, 0.80 mmol) and Pd(PPh₃)₄ (34 mg) in DME (3 mL). Purified by flash chromatography as a white solid (40 mg, 71%).

¹H NMR (CDCl₃, 400 MHz) δ (ppm) 8.54 (s, 2H), 8.07 (dd, J=1.2, 4.8, 1H), 7.45 (dd, J=1.6, 7.6 Hz, 1H), 6.95 (dd, J=4.8, 4.0 Hz, 1H), 3.92 (s, 3H). ¹³C NMR (CDCl₃, 100 MHz): 163.6, 160.3, 156.9, 143.9, 137.1, 130.4, 117.3, 113.8, 54.0.

HRMS (ES-MS) [(M+H)⁺]: for C₁₀H₈BrN₃O₂ 281.9873, found 281.9872.

### Example 58

### 1-Methyl-4-phenoxypyrimidin-2(1H)-one

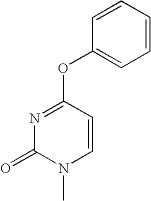

This compound was synthesized according to Example 18 from 3-(5-bromopyrimidin-2-yloxy)-3H-[1,2,3]triazolo[4,5-b]pyridine (50 mg, 0.20 mmol), phenylboronic acid (55 mg, 0.45 mmol), Cs₂CO₃ (260 mg, 0.80 mmol) and Pd(PPh₃)₄ (34 mg) in DME (3 mL). Purified by flash chromatography as a white solid (30 mg, 75%).

¹H NMR (CDCl₃, 400 MHz) δ (ppm) 7.55 (d, J=7.2 Hz, 1H), 7.39-7.35 (m, 2H), 7.23-7.20 (m, 1H), 7.15-7.12 (m, 2H), 6.05 (d, J=7.2 Hz, 1H), 3.49 (s, 3H). ¹³C NMR (CDCl₃, 100 MHz): 171.3, 156.4, 151.6, 148.6, 129.5, 125.8, 121.6, 95.0, 38.2.

HRMS (ES-MS) [(M+H)+]: for C₁₁H₁₀N₂O₃ 203.0815, found 203.0816.

### Example 59

### 4-(2-Methoxypyridin-3-yloxy)-1-methylpyrimidin-2(1H)-one

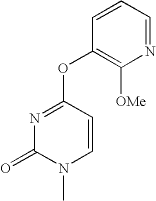

This compound was synthesized according to Example 18 from 3-(5-bromopyrimidin-2-yloxy)-3H-[1,2,3]triazolo[4,5-b]pyridine (50 mg, 0.20 mmol), methoxypyridin-3-ylboronic acid (68 mg, 0.45 mmol), Cs₂CO₃ (260 mg, 0.80 mmol) and Pd(PPh₃)₄ (34 mg) in DME (3 mL). Purified by flash chromatography as a white solid (40 mg, 86%).

¹H-NMR (CDCl₃, 400 MHz) δ (ppm) 8.04 (s, 2H), 7.60 (d, 2H, J=4.0 Hz), 7.39 (d, 2H, J=8.0 Hz), 6.91 (d, 2H, J=4.0 Hz), 6.14 (d, 2H, J=8.0 Hz), 3.93 (s, 3H), 3.49 (s, 3H).

HRMS (ES-MS) [(M+H)+]: for C₁₁H₁₁N₃O₃ 234.0873, found 234.0871.

### Example 60

### 4-(1-Methyl-2-oxo-1,2-dihydro-pyrimidin-4-yloxy)-benzoic Acid Methyl Ester

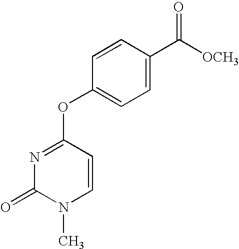

This compound was synthesized according to Example 40 from 1-methyl-4-([1,2,3]triazolo[4,5-b]pyridin-3-yloxy)-1H-pyrimidin-2-one (30 mg, 0.12 mmole), 4-(carboxymethyl)-phenyl boronic acid (66 mg, 0.37 mmole), Cs₂CO₃ (160 mg, 0.49 mmole), and Pd(PPh₃)₄ (21 mg, 0.02 mmole) in DME (2 mL) under oxygen atmosphere. The reaction mixture was purified by flash chromatography as a white solid (11 mg, 34%).

¹H-NMR (CDCl₃, 300 MHz) δ (ppm) 8.32 (d, 1H, J=7.8 Hz), 8.19 (d, 1H, J=8.7 Hz), 8.07 (d, 1H, J=8.1 Hz), 7.84 (d, 1H, J=8.1 Hz), 3.98 (s, 3H), 3.93 (s, 3H).

### Example 61

### 4-(4-Acetyl-phenoxy)-1-methyl-1H-pyrimidin-2-one

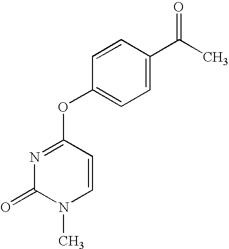

This compound was synthesized according to Example 40 from 1-methyl-4-([1,2,3]triazolo[4,5-b]pyridin-3-yloxy)-1H-pyrimidin-2-one (30 mg, 0.12 mmole), 4-acetyl-phenyl boronic acid (60 mg, 0.37 mmole), Cs₂CO₃ (160 mg, 0.49 mmole), and Pd(PPh₃)₄ (21 mg, 0.02 mmole) in DME (2 mL) under oxygen atmosphere. The reaction mixture was heated to 45° C. for 10 h and was purified by flash chromatography as a white solid (15 mg, 38%).

¹H-NMR (CDCl₃, 300 MHz) δ (ppm) 7.67 (m, 2H), 7.55 (m, 2H), 7.47 (m, 1H), 2.60 (s, 3H), 2.51 (s, 3H).

LCMS (ES-MS) [(M+Na)+]: for C₁₃H₁₂N₂O₃ 267.23, found 267.25.

### Example 62

### 4-(3-Acetyl-phenoxy)-1-methyl-1H-pyrimidin-2-one

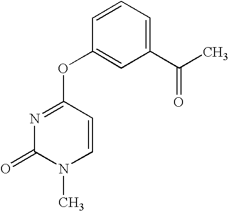

This compound was synthesized according to Example 40 from 1-methyl-4-([1,2,3]triazolo[4,5-b]pyridin-3-yloxy)-1H-pyrimidin-2-one (30 mg, 0.12 mmole), 3-acetyl-phenyl boronic acid (60 mg, 0.37 mmole), Cs₂CO₃ (160 mg, 0.49 mmole), and Pd(PPh₃)₄ (21 mg, 0.02 mmole) in DME (2 mL) under oxygen atmosphere. The reaction mixture was purified by flash chromatography as a white solid (10 mg, 33

¹H-NMR (CDCl₃, 300 MHz) δ (ppm) 7.52 (m, 2H), 7.34 (t, 1H, J=8.7 Hz), 7.10 (m, 1H), 6.54 (s, 1H), 2.60 (s, 3H), 2.06 (s, 3H).

### Example 63

### 4-(Pyrimidin-5-yloxy)quinazoline

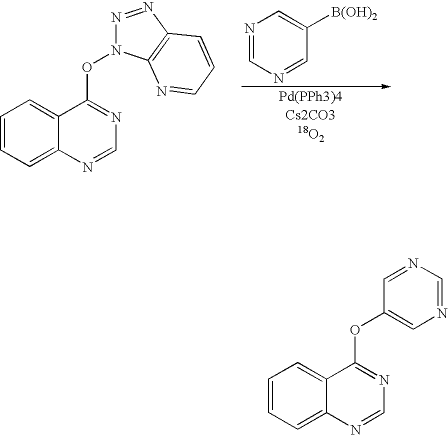

This compound was synthesized according to Example 31 from 4-(3H-[1,2,3]triazolo[4,5-b]pyridin-3-yloxy)quinazoline (50 mg, 0.19 mmol, pyrimidin-5-ylboronic acid (51 mg, 0.41 mmol), Cs₂CO₃ (247 mg, 0.76 mmol) and Pd(PPh₃)₄ (33 mg) in DME (3 mL) in the presence of ¹⁸O₂. The reaction was then monitored using ESI-LCMS. The disappearance of the starting material and the appearance of the product was followed with time. After 20 hours, the labeled oxygen was incorporated in the product in 42% relative yield with respect to 1602 incorporated product. Total conversion to 4-(pyrimidin-5-yloxy)quinazoline was 55%.

¹H NMR (CDCl₃, 400 MHz): δ (ppm) 9.06 (s, 1H), 8.41 (s, 2H), 8.77 (s, 1H), 8.39-8.37 (m, 1H), 8.06-7.96 (m, 2H), 7.76-7.73 (m, 1H). ¹³C NMR (CDCl₃, 100 MHz): δ (ppm) 166.0, 156.0, 153.8, 152.4, 151.2, 147.9, 135.0, 128.6, 128.5, 123.5, 116.1

MS (ESI-LCMS) [(M+H)⁺]: for C₁₂H₈N₄(18)O₂ 225.2, found 227.2.

Examples 64-77 (Table 1) and Examples 78-83 (Table 2) show preparation of representative compounds by a procedure similar to that of Scheme 4, but with the inclusion of water (0.8%) in the reaction mixture:

TABLE 1
Cross-Coupling Reaction of 3-(5-Bromo-pyrimidin-2-yloxy)-3H-
[1,2,3]triazolo[4,5-b]pyridine with Aryl Boronic Acid

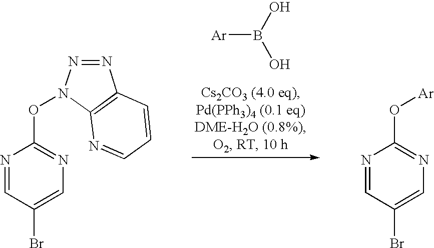

| | | Yield |
|---|---|---|
| Example | Boronic Acid | (%) |
| 64 | 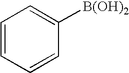 | 60 |
| 65 | 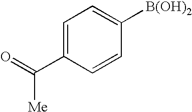 | 95 |
| 66 | 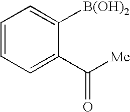 | 17 |
| 67 | 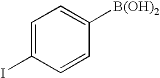 | 32 |
| 68 | 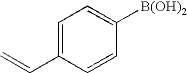 | 48 |
| 69 | 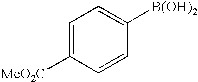 | 48 |
| 70 | 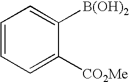 | 63 |
| 71 | 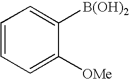 | 85 |
| 72 | 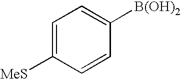 | 70 |
| 73 | 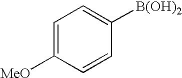 | 83 |
| 74 | 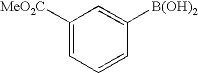 | 61 |
| 75 | 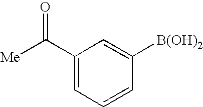 | 87 |
| 76 | 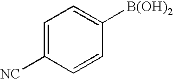 | 26 |
| 77 | 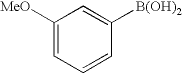 | 25 |

TABLE 2
Cross-Coupling Reaction of 3-(5-Bromo-pyrimidin-2-yloxy)-3H-
[1,2,3]triazolo[4,5-b]pyridine with Hetero-aryl Boronic Acid

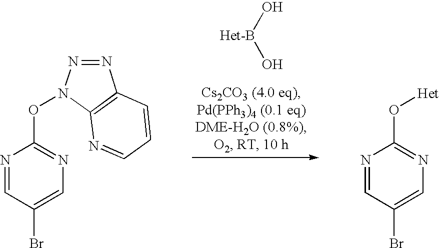

| | | Yield |
|---|---|---|
| Example | Boronic Acid | (%) |
| 78 | 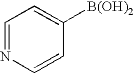 | 23 |
| 79 | 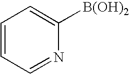 | 0 |
| 80 | 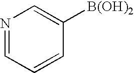 | 57 |
| 81 | 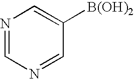 | 55 |
| 82 | 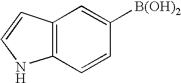 | 40 |
| 83 | 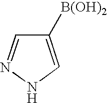 | 27 |

Examples 84-86 show the preparation of representative compounds by Suzuki coupling of 5-bromo-2-(4-methoxyphenoxy)pyrimidine with aryl boronic acid.

### Suzuki Coupling

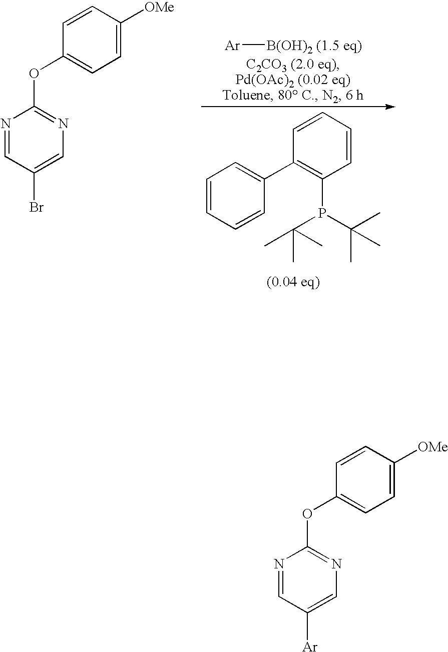

### Example 84

### 2-(4-methoxyphenoxy)-5-phenylpyrimidine

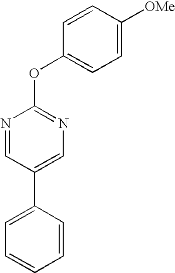

To a solution of 5-bromo-2-(4-methoxyphenoxy)pyrimidine (30 mg, 0.10 mmole) in toluene (2 mL) phenyl boronic acid (16 mg, 0.13 mmole) was added. Palladium catalyst Pd(OAc)₂ (0.5 mg, 0.002 mmole), phosphine ligand biphenyl-2-yldi-tert-butylphosphine (1.2 mg, 0.004 mmole) and Cs₂CO₃ (66 mg, 0.20 mmole) was added to the reaction mixture under nitrogen atmosphere. The reaction mixture was heated to 75° C. for 6 h under nitrogen atmosphere. The crude reaction mixture was purified by flash chromatography as a white solid (15 mg, 56%).

¹H NMR (CDCl₃, 400 MHz): δ (ppm) 8.75 (s, 2H), 7.82-7.44 (m, 5H), 7.18 (d, 2H, J=8.8 Hz), 6.89 (d, 2H, J=8.8 Hz), 3.84 (s, 3H).

LCMS (ES-MS) [(M+Na)+]: for C₁₇H₁₄N₂O₂ 279.31, found 279.50.

### Example 85

### 1-(4-(2-(4-methoxyphenoxy)pyrimidin-5-yl)phenyl)ethanone

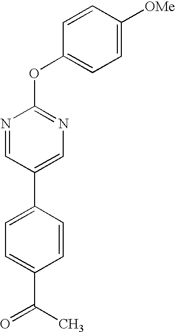

To a solution of 5-bromo-2-(4-methoxyphenoxy)pyrimidine (30 mg, 0.10 mmole) in toluene (1 mL) 4-acetyl phenyl boronic acid (26 mg, 0.16 mmole) was added. Catalyst Pd(OAc)₂ (0.5 mg, 0.002 mmole), biphenyl-2-yldi-tert-butylphosphine ligand (1.2 mg, 0.004 mmole) and Cs₂CO₃ (69 mg, 0.21 mmole) was added to the reaction mixture under nitrogen atmosphere. The reaction mixture was heated to 75° C. for 6 h under nitrogen atmosphere. The crude reaction mixture was then purified by flash chromatography as a white solid (14 mg, 41%).

LCMS (ES-MS) [(M+H)+]: for C₁₉H₁₆N₂O₃ 321.35, found 321.70.

### Example 86

### Methyl 4-(2-(4-methoxyphenoxy)pyrimidin-5-yl)benzoate

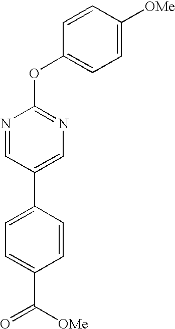

To a solution of 5-bromo-2-(4-methoxyphenoxy)pyrimidine (30 mg, 0.10 mmole) in toluene (1 mL) 4-carboxymethyl phenyl boronic acid (29 mg, 0.16 mmole) was added to it. Catalyst Pd(OAc)₂ (0.5 mg, 0.002 mmole), biphenyl-2-yldi-tert-butylphosphine ligand (1.2 mg, 0.004 mmole) and Cs₂CO₃ (69 mg, 0.21 mmole) was added to the reaction mixture under nitrogen atmosphere. The reaction mixture was heated to 75° C. for 6 h under nitrogen atmosphere. The reaction was purified by flash chromatography as a white solid (9 mg, 26%).

LCMS (ES-MS) [(M+H)+]: for C₁₉H₁₆N₂O₄ 337.35, found 337.50.

Examples 87-97 (Table 3) and Examples 98-101 (Table 4) show preparation of representative compounds according to Scheme 4, in which H₂O₂ is employed in place of oxygen (no Pd catalyst):

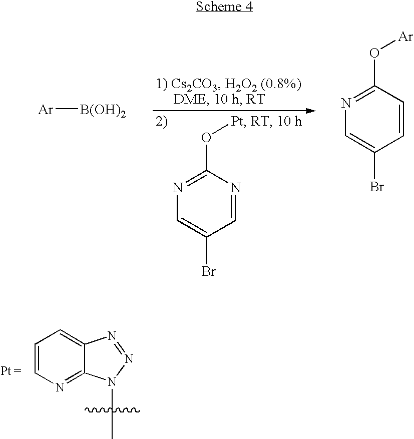

TABLE 3
Orthogonal Oxidative Coupling of Different
OPT Derived Derivatives (No Pd catalyst)

| | | Yield |
|---|---|---|
| Example | Boronic Acid | (%) |
| 87 | 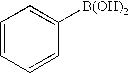 | 58 |
| 88 | 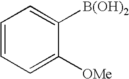 | 82 |
| 89 | 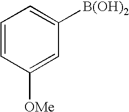 | 25 |
| 90 | 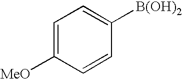 | 58 |
| 91 | 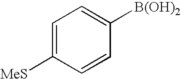 | 75 |
| 92 | 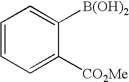 | 19 |
| 93 | 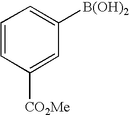 | 61 |
| 94 | 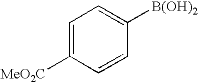 | 53 |
| 95 | 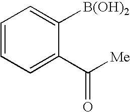 | 17 |
| 96 | 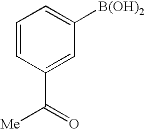 | 84 |
| 97 | 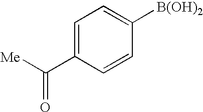 | 34 |

TABLE 4

| | | Yield |
|---|---|---|
| Example | Boronic Acid | (%) |
| 98 | 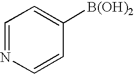 | 23 |
| 99 | 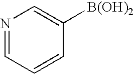 | 57 |
| 100 | 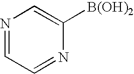 | 55 |
| 101 | 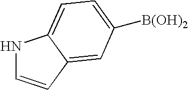 | 40 |

Examples 102-103 show preparation of a representative compound by the procedure of Scheme 5 below.

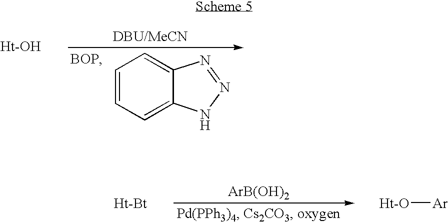

### Example 102

### 4-(1H-Benzo[d][1,2,3]triazol-1-yl)quinazoline

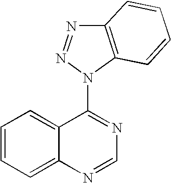

To a solution of a hydroxyquinazoline (292 mg, 0.5 mmol), and BOP (1060 mg, 2.4 mmol) was added DBU (0.45 mL, 3.0 mmol) at room temperature under nitrogen. The resultant mixture was stirred for 5-10 min at room temperature, after which benzotriazole (714 mg, 6.0 mmol) was added. The reaction mixture was monitored by LCMS till complete consumption of starting material (30 hrs). The solvent was removed under vacuum, the crude reaction mixture was purified by a flash chromatography on SiO₂ column eluted with hexanes/EtOAc to give the desired product (380 mg, 77%).

¹H NMR (DMSO-d6, 400 MHz): δ (ppm) 9.38 (s, 1H), 8.92 (d, J=8.4 Hz, 1H), 8.46 (d, J=8.0 Hz, 1H), 8.29 (d, J=8.0 Hz, 1H), 8.19-8.14 (m, 2H), 7.91 (t, J=6.8 Hz, 1H), 7.78 (t, J=7.6 Hz, 1H), 7.62 (t, J=7.6 Hz, 1H). ¹³C NMR (DMSO-d6,400 MHz): 155.0, 154.0, 153.0, 145.6, 135.4, 132.5, 130.2, 129.7, 128.8, 127.0, 126.3, 120.1, 117.2, 115.1.

HRMS (ES-MS) [(M+H)⁺]: for C₁₄H₉N₅ 248.0930, found 248.0930.

### Example 103

### 4-Phenoxyquinazoline

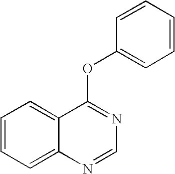

A suspension of 4-(1H-benzo[d][1,2,3]triazol-1-yl)quinazoline (58 mg, 0.23 mmol), phenyl boronic acid (78 mg, 0.5 mmol), Pd(PPh₃)₄ (40 mg) and Cs₂CO₃ (300 mg, 0.92 mmol) was heated at 45° C. for 24 hour under oxygen atmosphere. The solvent was removed under vacuum, the crude reaction mixture was purified by a flash chromatography on SiO₂ column eluted with hexanes/EtOAc to give the desired product (39 mg, 78%).

Examples 104-106 show preparation of representative compounds by the procedure of Scheme 6 below.

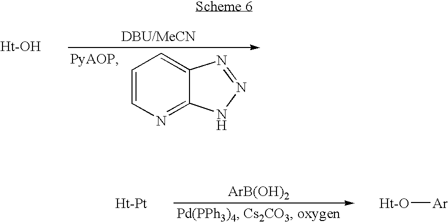

### Example 104

### 4-(3H-[1,2,3]Triazolo[4,5-b]pyridin-3-yl)quinazoline

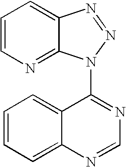

To a solution of a hydroxyquinazoline (400 mg, 2.73 mmol), and BOP (1450 mg, 3.2 mmol) was added DBU (0.61 mL, 4.09 mmol) at room temperature under nitrogen. The resultant mixture was stirred for 5-10 min at room temperature, after which 3H-[1,2,3 triazolo[4,5-b]pyridine (714 mg, 6.0 mmol) was added. The reaction mixture was monitored by LCMS till complete consumption of starting material (30 hrs). The solvent was removed under vacuum, the crude reaction mixture was purified by a flash chromatography on SiO₂ column eluted with hexanes/EtOAc to give the desired product (200 mg, 30%).

¹H-NMR (CDCl₃, 400 MHz) δ (ppm) 9.43 (s, 1H), 9.01 (d, 1H, J=8.4 Hz), 8.93 (m, 2H), 8.22-8.19 (m, 2H), 7.99-7.96 (m, 1H), 7.87 (dd, 1H, J=8.0 Hz, J=4.4 Hz).

HRMS (ES-MS) [(M+H)+]: for C₁₃H₈N₆ 248.2428, found 248.249.

### Example 105

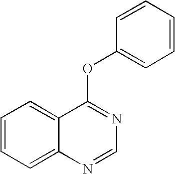

### 4-Phenoxyquinazoline

A suspension of 4-(1H-benzo[d][1,2,3]-triazol-1-yl)quinazoline (70 mg, 0.28 mmol), phenyl boronic acid (75 mg, 0.62 mmol), Pd(PPh₃)₄ (48 mg) and Cs₂CO₃ (400 mg, 1.23 mmol) was heated at 45° C. for 24 hour under oxygen atmosphere. The solvent was removed under vacuum, the crude reaction mixture was purified by a flash chromatography on SiO₂ column eluted with hexanes/EtOAc to give the desired product (35 mg, 56%).

### Example 106

### 4-(2-methylphenoxy)quinazoline

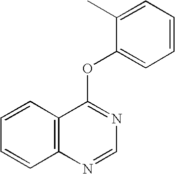

The subject compound can be prepared from 4-(1H-benzo[d][1,2,3]triazol-1-yloxy)quinazoline (Example 1) and 2-methylphenyl boronic acid using the procedure of Example 7; or from 4-(1H-benzo[d][1,2,3]triazol-1-yl)quinazoline and 2-methylphenyl boronic acid using the procedure of Example 101.

Examples 107-109 show preparation of representative compounds by the procedure of the second reaction of Scheme 7 below.

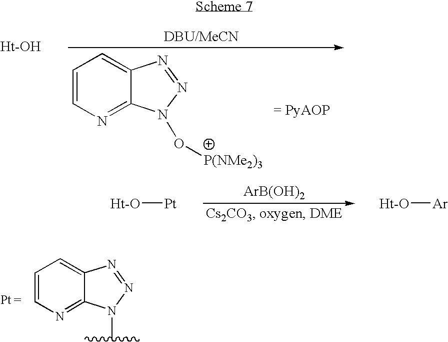

### Example 107

### 5-Bromo-2-phenoxy-pyrimidine

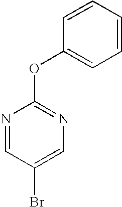

3-(5-Bromo-pyrimidin-2-yloxy)-3H-[1,2,3]triazolo[4,5-b]pyridine (20 mg, 0.07 mmole) was dissolved in DME (1 mL) at RT and Phenyl boronic acid (25 mg, 0.20 mmole) was added to it. Cs₂CO₃ (88 mg, 0.27 mmole) was added to the reaction mixture and purged with O₂. The reaction mixture was then stirred at RT for 10 h and was directly purified by flash chromatography to afford a white solid (4 mg, 24%).

¹H-NMR (CDCl₃, 300 MHz) δ (ppm) 8.57 (s, 2H), 7.44 (m, 2H), 7.26 (m, 1H), 7.17 (d, 2H, J=4.2 Hz).

LCMS (ES-MS) [(M+H)+]: for C₁₀H₇BrN₂O 251.07, found 251.30.

### Example 108

### 5-Bromo-2-(4-methoxy-phenoxy)-pyrimidine

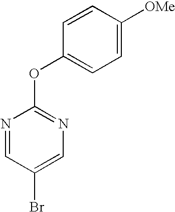

This compound was synthesized according to Example 103 from 3-(5-Bromo-pyrimidin-2-yloxy)-3H-[1,2,3]triazolo[4,5-b]pyridine (30 mg, 0.10 mmole), 4-methoxy phenyl boronic acid (47 mg, 0.31 mmole), Cs₂CO₃ (133 mg, 0.41 mmole) in DME (3 mL) and was purified by flash chromatography as a white solid (8 mg, 27%).

¹H-NMR (CDCl₃, 300 MHz) δ (ppm) 8.56 (s, 2H), 7.12 (d, 2H, J=9.3 Hz), 6.96 (d, 1H, J=9.0 Hz), 3.82 (s, 3H).

HRMS (ES-MS) [(M+H)+]: for C₁₁H₉BrN₂O₂ 280.9920, found 280.9919.

### Example 109

### 4-Phenoxy-quinazoline

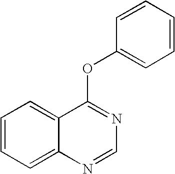

This compound was synthesized according to Example 103 from 4-([1,2,3]triazolo[4,5-b]pyridin-3-yloxy)-quinazoline (30 mg, 0.11 mmole), phenyl boronic acid (42 mg, 0.34 mmole), Cs₂CO₃ (148 mg, 0.46 mmole) in DME (3 mL) heated to 45° C. and was purified by flash chromatography as a white solid (12 mg, 47%).

¹H-NMR (CDCl₃, 300 MHz) δ (ppm) 8.78 (s, 1H), 8.39 (d, 2H, J=6.9 Hz), 8.03 (d, 2H, J=8.1 Hz), 7.93 (m, 1H), 7.68 (m, 1H), 7.53 (t, 2H, J=7.5 Hz), 7.34 (m, 2H),

LCMS (ES-MS) [(M+H)+]: for C₁₁H₉BrN₂O₂ 223.24, found 223.30.

Examples 110-111 show preparation of representative compounds by the procedure of the second reaction of Scheme 8 below.

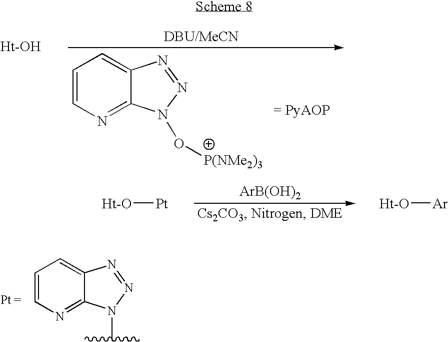

### Example 110

### 5-Bromo-2-phenoxy-pyrimidine

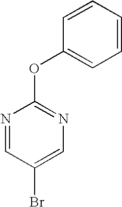

3-(5-Bromo-pyrimidin-2-yloxy)-3H-[1,2,3]triazolo[4,5-b]pyridine (10 mg, 0.03 mmole) was dissolved in DME (1 mL) at RT and phenyl boronic acid (12 mg, 0.10 mmole) was added to it. Cs₂CO₃ (44 mg, 0.14 mmole) was added to the reaction mixture and purged with N₂. The reaction mixture was then stirred at RT for 10 h and was directly purified by flash chromatography to afford a white solid (2 mg, 21%).

¹H-NMR (CDCl₃, 300 MHz) δ (ppm) 8.57 (s, 2H), 7.44 (m, 2H), 7.26 (m, 1H), 7.17 (d, 2H, J=4.2 Hz).

LCMS (ES-MS) [(M+H)+]: for C₁₀H₇BrN₂O 251.07, found 251.30.

### Example 111

### 5-Bromo-2-(4-methoxy-phenoxy)-pyrimidine

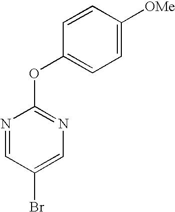

This compound was synthesized according to Example 106 from 3-(5-bromo-pyrimidin-2-yloxy)-3H-[1,2,3]triazolo[4,5-b]pyridine (30 mg, 0.10 mmole), 4-methoxy phenyl boronic acid (47 mg, 0.31 mmole), Cs₂CO₃ (133 mg, 0.41 mmole) in DME (3 mL) under a nitrogen atmosphere and was purified by flash chromatography as a white solid (15 mg, 51%).

¹H-NMR (CDCl₃, 300 MHz) δ (ppm) 8.56 (s, 2H), 7.12 (d, 2H, J=9.3 Hz), 6.96 (d, 1H, J=9.0 Hz), 3.82 (s, 3H).

HRMS (ES-MS) [(M+H)+]: for C₁₁H₉BrN₂O₂ 280.9920, found 280.9919.

Examples 112-113 show preparation of representative compounds by the procedure of Scheme 9 below.

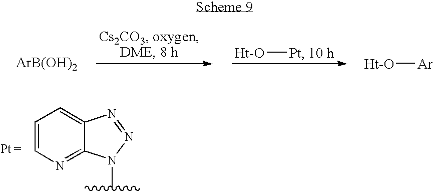

### Example 112

### 5-Bromo-2-phenoxy-pyrimidine

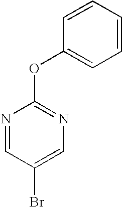

Phenyl boronic acid (100 mg, 0.82 mmole) was dissolved in DME (6 mL) and Cs₂CO₃ (346 mg, 1.06 mmole) was added to the reaction mixture and purged with O₂. The reaction mixture was stirred for 8 h at RT. 3-(5-bromo-pyrimidin-2-yloxy)-3H-[1,2,3]triazolo[4,5-b]pyridine (60 mg, 0.25 mmole) was added at RT. The reaction mixture was then stirred at RT for 10 h and was directly purified by flash chromatography to afford a white solid (25 mg, 60%, brsm).

¹H-NMR (CDCl₃, 300 MHz) δ (ppm) 8.57 (s, 2H), 7.44 (m, 2H), 7.26 (m, 1H), 7.17 (d, 2H, J=4.2 Hz).

LCMS (ES-MS) [(M+H)+]: for C₁₀H₇BrN₂O 251.07, found 251.30.

### Example 113

### 5-Bromo-2-(4-methoxy-phenoxy)-pyrimidine

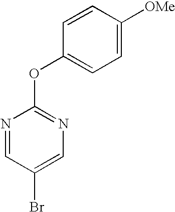

This compound was synthesized according to Example 108 from 4-methoxy phenyl boronic acid (150 mg, 0.98 mmole), Cs₂CO₃ (417 mg, 1.28 mmole) and 3-(5-bromo-pyrimidin-2-yloxy)-3H-[1,2,3]triazolo[4,5-b]pyridine (72 mg, 0.25 mmole), in DME (7 mL) under an oxygen atmosphere and was purified by flash chromatography as a white solid (58 mg, 83%).

¹H-NMR (CDCl₃, 300 MHz) δ (ppm) 8.56 (s, 2H), 7.12 (d, 2H, J=9.3 Hz), 6.96 (d, 1H, J=9.0 Hz), 3.82 (s, 3H).

HRMS (ES-MS) [(M+H)+]: for C₁₁H₉BrN₂O₂ 280.9920, found 280.9919.

Examples 114-125 (Table 5) show preparation of representative compounds according to Scheme 10, in which 2,4-di-OPT pyrimidine is selectively cross coupled (H₂O₂; no Pd catalyst):

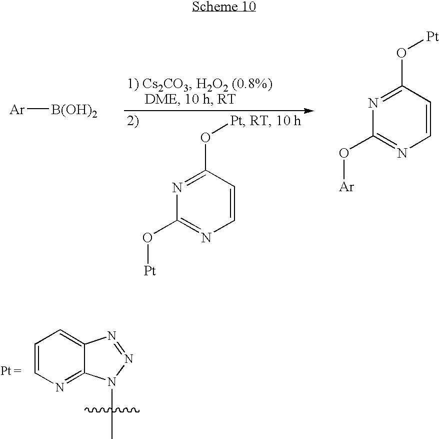

TABLE 5
Orthogonal Oxidative Coupling of Di-OPT Pyrimidine
(No Pd catalyst)

| | | Yield |
|---|---|---|
| Example | Boronic Acid | (%) |
| 114 | 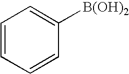 | 37 |
| 115 | 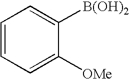 | 28 |
| 116 | 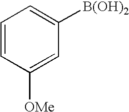 | 34 |
| 117 | 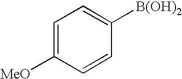 | 47 |
| 118 | 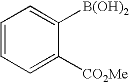 | 28 |
| 119 | 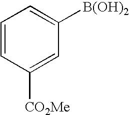 | 33 |
| 120 | 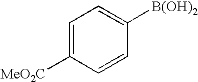 | 36 |
| 121 | 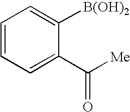 | 41 |
| 122 | 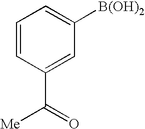 | 39 |
| 123 | 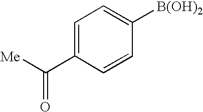 | 52 |
| 124 | 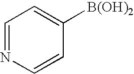 | 34 |
| 125 | 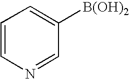 | 28 |

Examples 126-129 (Table 6) show preparation of representative compounds according to Scheme 11, in which 2,4-di-OPT pyrimidine is selectively cross coupled (O₂, Pd catalyst):

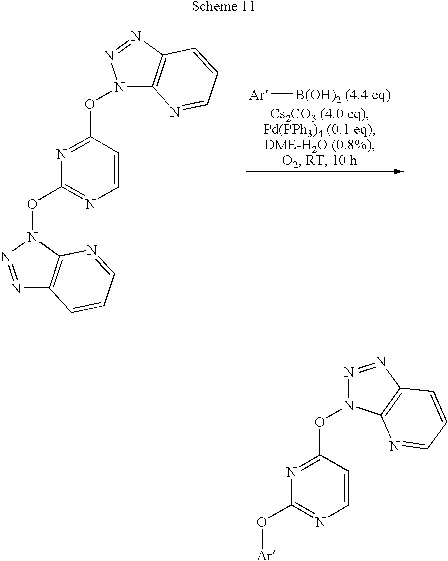

TABLE 6
Orthogonal Cross Coupling of Di-OPT Pyrimidine (Pd catalyst)

| Example | Ar′—B(OH)₂ | Yield |
|---|---|---|
| 126 | 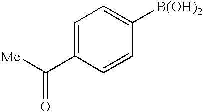 | 82 |
| 127 | 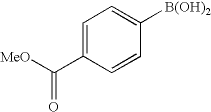 | 61 |
| 128 | 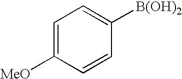 | 52 |
| 129 | 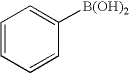 | 67 |

Examples 130-134 (Table 7) show preparation of representative compounds according to Scheme 12, in which 2,4-di-OPT pyrimidine is non-selectively cross coupled (no O₂, H₂O₂ or Pd catalyst):

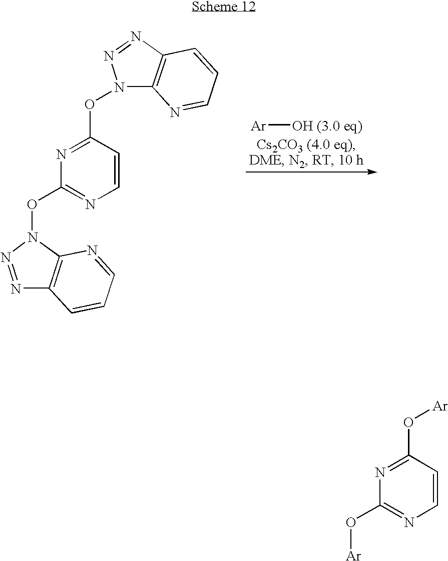

TABLE 7
Addition of Phenols to form 3-[2-(3H-[1,2,3]triazolo
[4,5-b]pyridine)-pyrimidin-4-
yloxy]-3H-[1,2,3]triazolo[4,5-b]pyridine (No Pd catalyst)

| Example | Ar—OH | Yield |
|---|---|---|
| 130 | 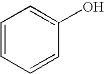 | 62% |
| 131 | 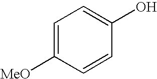 | 69% |
| 132 | 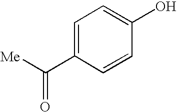 | 57% |
| 133 | 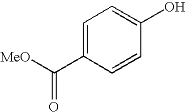 | 57% |
| 134 | 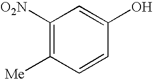 | 36% |

Examples 135-138 (Table 8) show preparation of representative compounds according to Scheme 13, in which phenols are added to 3-[2-(3H-[1,2,3]triazolo[4,5-b]pyridine)-pyrimidin-4-yloxy]-3H-[1,2,3]triazolo[4,5-b]pyridine (no O₂, H₂O₂ or Pd catalyst):

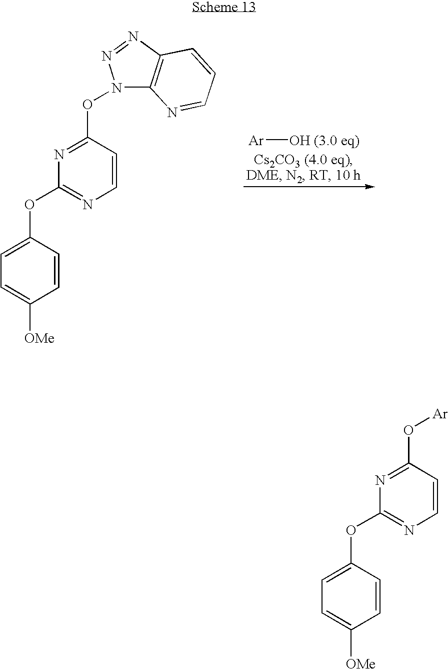

TABLE 8
Addition of Phenols to 3-[2-(3H-[1,2,3]triazolo
[4,5-b]pyridine)-pyrimidin-4-
yloxy]-3H-[1,2,3]triazolo[4,5-b]pyridine (No Pd catalyst)

| Example | Ar—OH | Yield |
|---|---|---|
| 135 | 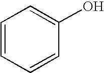 | 75% |
| 136 | 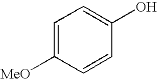 | 58% |
| 137 | 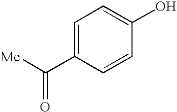 | 47% |
| 138 | 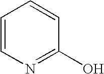 | No Product |

### Biological Test Procedures and Results

### PI3 Kinase Assay

PI3-Kinase reactions were performed in 5 μM HEPES, pH 7, 2.5 μM MgCl2, and 25 μM ATP, with diC8-PI(4,5)P2 (Echelon, Salt Lake City Utah) as substrate. Nunc 384 well black polypropylene fluorescent plates were used for PI3K assays. Reactions were quenched by the addition of EDTA to a final concentration of 10 μM. Final reaction volumes were 10 ml. For evaluation of PI 3-K inhibitors, 5 ng of enzyme and 2.5 μM of substrate was used per 10 ml reaction volume, and inhibitor concentrations ranged from 100 μM to 20 μM; the final level of DMSO in reactions never exceeded 2%. Reactions were allowed to proceed for one hour at 25° C. After 1 hour, GST-tagged GRP1 (general receptor for phosphoinositides) PH domain fusion protein was added to a final concentration of 100 nM, and BODIPY-TMRI(1,3,4,5)P4 (Echelon) was also added to a final concentration of 5 nM. Final sample volumes were 25 μl with a final DMSO concentration of 0.8%. Assay Plates were read on Perkin-Elmer Envision plate readers with appropriate filters for Tamra [BODIPY-TMRI(1,3,4,5)P4]. Data obtained were used to calculate enzymatic activity and enzyme inhibition by inhibitor compounds.

| | | PI3K alpha, % | PI3K gamma, |
|---|---|---|---|
| | | Inhibition at | % Inhibition at |
| Example # | Compound Name | 30uM | 30M |
| 44 | 4-(1H-Benzo[d][123]triazol-1-yl)quinazoline | 72% | 44% |
| 20 | 4-(4-Methoxyphenoxy)quinazoline | 65% | 4% |
| 21 | 4-(4-Trifluoromethyl)phenoxy)quinazoline | 36% | 6% |
| 19 | 4-(3-Nitrophenoxy)quinazoline | 24% | <10% |
| 22 | 1-(4-(Quinazolin-4-yloxy)phenyl)ethanone | 34% | 45% |
| 25 | 4-(6-Chloropyridin-3-yloxy)quinazoline | 42% | 11% |
| 24 | 4-(Quinazolin-4-yloxy)benzonitrile | 18% | <10% |
| 57 | 5-Bromo-2-(2-methoxypyridin-3-yloxy)pyrimidine | 44% | <10% |
| 59 | 4-(2-Methoxypyridin-3-yloxy-methylpyrimidin-2(1H)-one | 46% | <10% |
| 58 | 1-Methyl-4-phenoxypyrimidin-2(1H)-one | 32% | <10% |
| 38 | Methyl 4-(3-cyanophenoxy)-5-methylthieno[2,3- | 40% | 23% |
| | d]pyrimidine-6-carboxylate | | |
| 37 | Methyl 5-methyl-4-(3-itrophenoxy)thieno[2,3- | 24% | 16% |
| | d]pyrimidine-6-carboxylate | | |
| 36 | Methyl 5-methyl-4-phenoxythieno[2,3-d]pyrimidine-6- | 63% | 36% |
| | carboxylate | | |
| 39 | 4-(3,5-dimethylisoxazol-4-yloxy-5-methylthieno[2,3- | 24% | 15% |
| | d]pyrimidine-6-carboxylate | | |
| 32 | 3-(Thieno[2,3-d]pyrimidin-4-yloxy)benzonitrile | 32% | <10% |
| 34 | 4-(3-Nitrophenoxy)thieno[2,3-d]pyrimidine | 14% | <10% |
| 35 | 3,5-Dimethyl-4-(thieno[2,3-d]pyrimidin-4- | 26% | 11% |
| | yloxy)isoxazole | | |
| 28 | 4-(2-Methoxypyridin-3-yloxy)quinazoline | 22% | <10% |
| 29 | 4-(1H-Indol-5-yloxy)quinazoline | 68% | 21% |
| 26 | 3,5-Dimethyl-4-(quinazolin-4-yloxy)isoxazole | 56% | 12% |
| 27 | 4-(1-Benzyl-1H-pyrazol-3-yloxy)quinazoline | 43% | 28% |
| 30 | 4-(Thiophen-3-yloxy)quinazoline | 87% | 55% |
| 31 | 4-(Pyrimidin-5-yloxy)quinazoline | 33% | 53% |

mTOR Enzyme Assay

The routine human TOR assays with purified enzyme were performed in 96-well plates by DELFIA format as follows. Enzymes were first diluted in kinase assay buffer (10 mM Hepes (pH 7.4), 50 mM NaCl, 50 mM β-glycerophosphate, 10 mM MnCl₂, 0.5 mM DTT, 0.25 μM microcystin LR, and 100 μg/mL BSA). To each well, 12 μL of the diluted enzyme were mixed briefly with 0.5 μL test inhibitor or control vehicle dimethylsulfoxide (DMSO). The kinase reaction was initiated by adding 12.5 μL kinase assay buffer containing ATP and His6-S6K to give a final reaction volume of 25 μL containing 800 ng/mL FLAG-TOR, 100 μM ATP and 1.25 μM His6-S6K. The reaction plate was incubated for 2 hours (linear at 1-6 hours) at room temperature with gentle shaking and then terminated by adding 25 μL Stop buffer (20 mM Hepes (pH 7.4), 20 mM EDTA, 20 mM EGTA). The DELFIA detection of the phosphorylated (Thr-389) His6-S6K was performed at room temperature using a monoclonal anti-P(T389)-p70S6K antibody (1A5, Cell Signaling) labeled with Europium-N-1-ITC (Eu) (10.4 Eu per antibody, PerkinElmer). The DELFIA Assay buffer and Enhancement solution were purchased from PerkinElmer. 45 μL of the terminated kinase reaction mixture was transferred to a MaxiSorp plate (Nunc) containing 55 μL PBS. The His6-S6K was allowed to attach for 2 hours after which the wells were aspirated and washed once with PBS. 100 [symbol] L of DELFIA Assay buffer with 40 ng/mL Eu-P(T389)-S6K antibody was added. The antibody binding was continued for 1 hour with gentle agitation. The wells were then aspirated and washed 4 times with PBS containing 0.05% Tween-20 (PBST). 100 [symbol] L of DELFIA Enhancement solution was added to each well and the plates were read in a PerkinElmer Victor model plate reader. Data obtained were used to calculate enzymatic activity and enzyme inhibition by potential inhibitors. Example 31, 4-(Pyrimidin-5-yloxy)quinazoline, 12% inhibition at 10 uM.

IKK Beta

Human IKKβ cDNA is amplified by reverse transcriptase-polymerase chain reaction from human placental RNA (CLONTECH) using primers that incorporated the FLAG-epitope at the C terminus of IKKβ. FLAG-IKKβ is inserted into the baculovirus expression plasmid pFASTBAC (Life Technologies). Following the manufacturer's protocol for the BAC-TO-BAC (Life Technologies) Baculovirus Expression System, recombinant baculoviruses expressing the IKKβ enzyme are made. Briefly, 9×10⁵ SF9 cells per well of a 6-well plate are transfected with one μg of miniprep bacmid DNA using the CellFECTIN™ reagent. Virus is harvested 72 hours post transfection, and a viral titer is performed, after which a high titer viral stock (2×10⁸ pfu/ml) is amplified by three to four rounds of infection.

### Flag-IKKβ Protein Production and Purification

Using the high titer stock of baculovirus expressing the Flag-IKKβ, 200 mL of SF9 cells at a density of 1×10⁶ cells/mL are infected at a multiplicity of infection (MOI) of approximately 5 at 27° C. in SF-900 II SFM medium. Cells are harvested 48-54 hours later by centrifugation at 500×g in a Sorvall centrifuge. The resulting pellets are frozen at −20° C. until purification.

For protein purification, the pellets are thawed on ice and resuspended in cell lysis buffer (50 mM HEPES, pH 7.5, 100 mM NaCl, 1% NP-40, 10% glycerol, 1 mM Na₃VO₄, 1 mM EDTA, 1 mM DTT, and protease inhibitor cocktail from Pharmingen). After Dounce homogenization, the cells are put in the cold room on a rotator for 30 minutes. The NaCl concentration is adjusted to 250 mM and the cell debris is removed by centrifugation at 18000×g. The resulting supernatant is loaded onto an anti-FLAG M2 agarose affinity column (Sigma) at 4° C. and the column is washed with 60 mL of wash buffer (50 mM HEPES, pH 7.5, 300 mM NaCl, 10% glycerol, 1 mM Na₃VO₄, 1 mM EDTA, and 1 mM PMSF). The FLAG-IKKβ is eluted using 200 μg/mL Flag peptide (Sigma) in elution buffer (50 mM HEPES, pH 7.5, 100 mM NaCl, 10% glycerol, 1 mM Na₃VO₄, 1 mM EDTA, 1 mM DTT, and protease inhibitor cocktail from Pharmingen) in 500 μL aliquots, which are tested for protein levels using SDS-PAGE followed by Coomassie Blue staining (BioRad). After testing for activity as described below, fractions with high IKK enzyme activity are combined, aliquoted, and stored at −80° C.

### IKK Kinase Assay

LANCE reactions are carried out based upon the suggestions of Wallac/Perkin Elmer. Purified Flag-IKKβ enzyme (2 nM final concentration) is typically used in the kinase reaction buffer described above supplemented with 0.0025% Brij solution (Sigma) to help stabilize the enzyme. Biotinylated substrate IκBα (1-54) is synthesized and purified (>95% pure) and is used at 500 nM final concentration. ATP is used at a final concentration of 2 μM. The total reaction volumes are 25 μL and the inhibitor compounds are preincubated with enzyme before substrate and ATP are added. Reactions are conducted for 30 minutes at room temperature in black, low binding plates (Dynex). 25 μL of 20 mM EDTA is added to terminate the reactions and then 100 μL of detection mixture [0.25 nM Europium labeled anti-phospho-IκBα (prepared by Wallac) and 0.25 μg/mL final concentration streptavidin-APC, Wallac] is added 30 minutes before reading the plates in a Wallac VICTOR plate reader. The energy transfer signal data is used to calculate percent inhibition and IC₅₀ values.

The compound of Example 26 (3,5-Dimethyl-4-(quinazolin-4-yloxy)isoxazole) displayed 51% inhibition at 30 uM.

### Braf

Reagents: Flag/GST-tagged recombinant human B-Raf produced in Sf21 insect cells (purchased from Upstate), human Mek-1-GST, non-active recombinant protein produced in E. coli (from Upstate); and a phospho-MEK1 specific poly-clonal Ab from Cell Signaling Technology (cat. #9121).

### B-Raf1 Kinase Assay Procedure

B-Raf-1 is used to phosphorylate GST-MEK1. MEK1 phosphorylation is measured by a phospho-specific antibody (from Cell Signaling Technology, cat. #9121) that detects phosphorylation of two serine residues at positions 217 and 221 on MEK1.

Compounds of the invention were assayed according to the following protocol.

### Kinase Assay Protocol

### B-Raf Assay Stock Solutions:

1. Assay Dilution Buffer (ADB): 20 mM MOPS, pH 7.2, 25 mM β-glycerol phosphate, 5 mM EGTA, 1 mM sodium orthovanadate, 1 mM dithiothreitol, 0.01% Triton X-100.
2. Magnesium/ATP Cocktail: ADB solution (minus Triton X-100) plus 200 μM cold ATP and 40 mM magnesium chloride.
3. Active Kinase: Active B-Raf: used at 0.2 nM per assay point.
4. Non-active GST-MEK1: Use at 2.8 nM final concentration).
5. TBST-Tris (50 mM, pH 7.5), NaCl (150 mM), Tween-20 (0.05%)

### 6. Anti-GST Ab (GE)

7. Anti pMEK Ab (Upstate)
8. Anti-rabbit Ab/Europium conjugate (Wallac)

### Assay Procedure:

1. Add 25 μl of ADB containing B-Raf and Mek per assay (i.e. per well of a 96 well plate)
2. Add 25 μl of 0.2 mM ATP and 40 mM magnesium chloride in Magnesium/ATP Cocktail.
3. Incubate for 45 minutes at RT with occasional shaking.
4. Transfer this mixture to an anti-GST Ab coated 96 well plate (Nunc Immunosorb plates coated o/n with a-GST. Plate freshly washed 3× with TBS-T before use.
5. Incubate o/n at 30° C. in cold room.
6. Wash 3× with TBST, add Anti-Phospho MEK1 (1:1000, dilution depends upon lot)
7. Incubate for 60 minutes at RT in a shaking incubator
8. Wash 3× with TBST, add Anti-rabbit Ab/Europium conjugate (Wallac) (1:500, dilution depends upon lot)
9. Incubate for 60 minutes at RT on a platform shaker.
10. Wash plate 3× with TBS-T
11. Add 100 ul of Wallac Delfia Enhancement Solution and shake for 10 minutes.
12. Read plates in Wallac Victor model Plate Reader.
13. Collect data analyze in Excel for single point and IC50 determinations.

The compound of Example 29 (4-(1H-Indol-5-yloxy)quinazoline) displayed 21% inhibition at 10 μM.

The compound of Example 30 (4-(Thiophen-3-yloxy)quinazoline) displayed 14% inhibition at 10 μM.

The compound of Example 58 (1-Methyl-4-phenoxypyrimidin-2(1H)-one) displayed 17% inhibition at 10 μM

The compound of Example 25 (4-(6-Chloropyridin-3-yloxy)quinazoline) displayed 20% inhibition at 10 μM.

### MK2

MK2 kinase activity was assessed using human recombinant MK2 (25 nM) containing residues 41 through 353 in an ELISA based assay. The kinase reaction was performed on 96-well streptavidin coated plates using a biotinylated 16-mer peptide as a substrate derived from LSP1 in 20 mM Hepes pH7.4, 10 mM MgCl2, 3 mM DTT, 1 uM ATP, 0.01% Triton X100, 2% DMSO and various compound concentrations in a final volume of 100 ul. The reaction was stopped after 30 min incubation at room temperature with 50 ul of 0.5M EDTA and washed 6 times in PBS 0.05% Tween 20. Polyclonal anti phospho-LSP1 antibodies was then added to the plate along with a Goat anti-Rabbit antibody labeled with europium in 20 mM MOPS, 150 mM NaCl, 0.025% Tween 20, 0.02% gelatin, 1% BSA for 1 hour at room temperature. The plate was then washed 6 times in PBS 0.05% Tween 20 and enhancement solution from Perkin Elmer was added before counting on a Victor 2 reader from Perkin Elmer.

The compound of Example 25 (4-(6-Chloropyridin-3-yloxy)quinazoline) displayed 31% inhibition at 30 μM.

The compound of Example 39 (4-3,5-dimethylisoxazol-4-yloxy)-5-methylthieno[2,3-d]pyrimidine-6-carboxylate) displayed 32% inhibition at 30 μM.

The compound of Example 30 (4-(Thiophen-3-yloxy)quinazoline) displayed 34% inhibition at 30 μM.

### IRAK4

IRAK4 kinase activity was assessed using human recombinant IRAK4 (4 nM) containing residues 154 through 460 in an ELISA based assay. The kinase reaction was performed on 96-well streptavidin coated plates using a biotinylated 11-mer peptide derived from MK2 in 20 mM Hepes pH7.4, 10 mM MgCl2, 3 mM DTT, 600 uM ATP, 0.01% Triton X100, 5% DMSO and various compound concentrations in a final volume of 100 ul. The reaction was stopped after 60 min incubation at room temperature with 50 ul of 0.5M EDTA and washed 6 times in PBS 0.05% Tween 20. A Rabbit polyclonal anti-phospho-threonine antibody was then added to the plate along with a Goat anti-Rabbit antibody labeled with europium in 20 mM MOPS, 150 mM NaCl, 0.025% Tween 20, 0.02% gelatin, 1% BSA for 1 hour at room temperature. The plate was then washed 6 times in PBS 0.05% Tween 20 and enhancement solution from Perkin Elmer was added before counting on a Victor 2 reader from Perkin Elmer.

The compound of Example 25 (4-(6-Chloropyridin-3-yloxy)quinazoline) displayed 19% inhibition at 10 μM.

The compound of Example 39 (4-3,5-dimethylisoxazol-4-yloxy)-5-methylthieno[2,3-d]pyrimidine-6-carboxylate) displayed 21% inhibition at 10 μM.

The compound of Example 30 (4-(Thiophen-3-yloxy)quinazoline) displayed 39% inhibition at 10 μM.

### PKC Theta IMAP Assay

The materials used include the following: human PKCθ full length enzyme (Panvera Cat#P2996); substrate peptide: 5FAM-RFARKGSLRQKNV-OH (Molecular Devices, RP7032); ATP (Sigma Cat #A2383); DTT (Pierce, 20291); 5× kinase reaction buffer (Molecular Devices, R7209); 5× binding buffer A (Molecular Devices, R7282), 5× binding buffer B (Molecular Devices, R7209); IMAP Beads (Molecular Devices, R7284); and 384-well plates (Corning Costar, 3710).

The reaction buffer was prepared by diluting the 5× stock reaction buffer and adding DTT to obtain a concentration of 3.0 mM. The binding buffer was prepared by diluting the 5× binding buffer A. A master mix solution was prepared using a 90% dilution of the reaction buffer containing 2×ATP (12 uM) and 2× peptide (200 nm). Compounds were diluted in DMSO to 20× of the maximum concentration for the IC50 measurement. 27 μl of the master mix solution for each IC50 curve was added to the first column in a 384-well plate and 3 μl of 20× compound in DMSO was added to each well. The final concentration of compound was 2× and 10% DMSO. DMSO was added to the rest of the master mix to increase the concentration to 10%. 10 μl of the master mix containing 10% DMSO was added to the rest of the wells on the plate except the 2nd column. 20 μl was transferred from the first column to the 2nd column. The compounds were serially diluted in 2:1 ratio starting from the 2nd column. A 2× (2 nM) PKCθ solution was made in the reaction buffer. 10 μl of the PKCθ solution was added to every well to achieve these final concentrations: PKCθ —1 nM; ATP—6 μM; peptide—100 nM; DMSO—5%. Samples were incubated for 25 minutes at room temperature. The binding reagent was prepared by diluting the beads in 1× binding buffer to 800:1. 50 μl of the binding reagent was added to every well and incubated for 20 minutes. FP was measured using Envision2100 (PerkinElmer Life Sciences). Wells with no ATPs and wells with no enzymes were used as controls.

The compound of Example 26 (3,5-Dimethyl-4-(quinazolin-4-yloxy)isoxazole) displayed an IC₅₀=25.9 μM.

Those skilled in the art will recognize that various changes and/or modifications may be made to aspects or embodiments of this invention and that such changes and/or modifications may be made without departing from the spirit of this invention. Therefore, it is intended that the appended claims cover all such equivalent variations as will fall within the spirit and scope of this invention.

It is intended that each of the patents, applications, and printed publications, including books, mentioned in this patent document be hereby incorporated by reference in their entirety.

What is claimed is:

1. A synthetic process comprising:
reacting a compound of Formula II:
Ht-(O)ₖ-L II
or a salt thereof, wherein:
k is 0 or 1;
L is a group having the Formula: 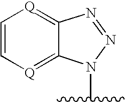
wherein one Q is CH, and one Q is CH or N;
Ht is a heterocycle of Formula a, b, c or d: 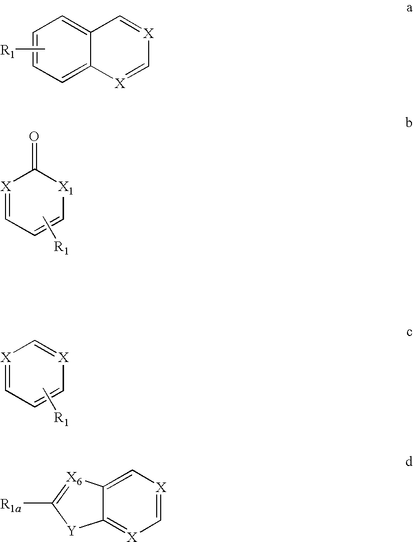
R₁, R₁ₐ and R_{1b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;
each X is independently N or CH;
X₆ is CR_{1b} or N;
X₁ is NH or CH₂; and
Y is S or O;
with a compound of Formula Ar—B(OH)₂; wherein Ar has one of the Formulas e-j: 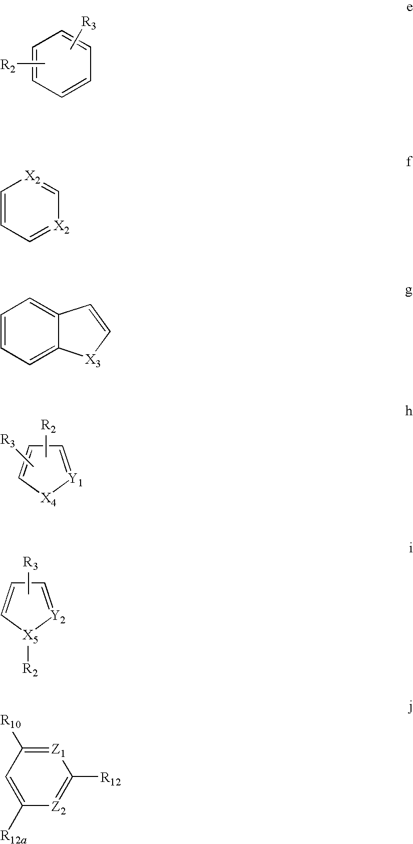
wherein:
each X₂ is independently N or CH;
X₃ is NH or CH₂;
X₄ is NH, S or O;
X₅ is N or CH;
Y₁ is N or CH;
Y₂ is N or CH;
R₂ and R₃ are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;
Z₁ is CR₁₁ₐ or N;
Z₂ is CR_{11b} or N; and
R₁₀, R₁₁ₐ, R_{11b}, R₁₂ and R₁₂ₐ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;
wherein the reaction is performed in the presence of a base;
and optionally in the presence of water;
and optionally in the presence of one or more of:
(i) oxygen;
(ii) a Pd(0) catalyst; or
(iii) H₂O₂;
for a time and under conditions effective to form a compound of Formula Ht-O—Ar.

2. The process of claim 1, wherein the reacting is performed in the presence of both oxygen and a palladium (0) catalyst.

3. The process of claim 1 or claim 2, wherein the palladium (0) catalyst comprises one or more of bis[1,2-bis(diphenylphosphino)ethane]palladium(0), bis(dibenzylideneacetone) palladium(0), 1,3-Bis(2,6-diisopropylphenyl)imidazol-2-ylidene(1,4-naphthoquinone)palladium(0) dimer, bis(3,5,3′,5′-dimethoxydibenzylideneacetone) palladium(0), bis(tri-tert-butylphosphine)palladium(0), 1,3-bis(2,4,6-trimethylphenyl)imidazol-2-ylidene (1,4-naphthoquinone)palladium(0) dimer, tetrakis(methyldiphenyl phosphine)palladium(0), tetrakis(triphenylphosphine)palladium(0), tris(dibenzylidene acetone)dipalladium(0), tris(dibenzylideneacetone)dipalladium(0)-chloroform adduct, and tris(3,3′,3″-phosphinidynetris(benzenesulfonato)palladium(0) nonasodium salt nonahydrate.

4. The process of claim 3, wherein the palladium (0) catalyst comprises tetrakis(triphenylphosphine)palladium(0); Pd(PPh₃)₄.

5. The process of claim 1, wherein the reacting is performed in the presence of H₂O₂.

6. The process of claim 1, wherein k is 0.

7. The process of claim 1, wherein k is 1.

8. The process of claim 1, wherein each Q is CH.

9. The process of claim 1, wherein one Q is N.

10. The process of claim 6, wherein the compound of Formula II is prepared by reacting a compound of Formula Ht-OH with a coupling reagent in the presence of a base, and under an inert atmosphere.

11. The process of claim 10, wherein the coupling reagent comprises a phosphonium salt having a cation of Formula: 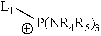
wherein:
L₁ is a moiety of Formula: 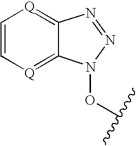
Q is one Q is CH, and one Q is CH or N;
each R₄ and each R₅ is independently C₁₋₆ alkyl;
and wherein any R₄ and R₅ attached to the same nitrogen atom can together form a moiety of formula —(CH₂)_{q}— where q is 2, 3, 4, 5 or 6.

12. The process of claim 11, wherein each Q is CH.

13. The process of claim 12, wherein said reacting of said compound of Formula Ht-OH with said coupling reagent is performed in the presence of benzotriazole.

14. The process of claim 11, wherein one Q is N.

15. The process of claim 14, wherein said reacting of said compound of Formula Ht-OH with said coupling reagent is performed in the presence of 3H-[1,2,3]triazolo[4,5-b]pyridine.

16. The process of claim 10, wherein said coupling reagent is BOP.

17. The process of claim 16, wherein said reacting of said compound of Formula Ht-OH with said coupling reagent is performed in the presence of benzotriazole.

18. The process of claim 10, wherein the coupling reagent is PyAOP.

19. The process of claim 18, wherein said reacting of said compound of Formula Ht-OH with said coupling reagent is performed in the presence of 3H-[1,2,3]triazolo[4,5-b]pyridine.

20. The process of claim 7, wherein the compound of Formula II is prepared by reacting a compound of Formula Ht-OH with coupling reagent in the presence of a base, and in the presence of oxygen.

21. The process of claim 20, wherein the compound of Formula II is prepared by reacting a compound of Formula Ht-OH with coupling reagent in the presence of a base, and in the presence of air.

22. The process of claim 20, wherein the coupling reagent comprises a phosphonium salt having a cation of Formula: 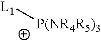
wherein:
L₁ is a moiety of Formula: 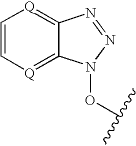
one Q is CH, and one Q is CH or N;
each R₄ and each R₅ is independently C₁₋₆ alkyl;
and wherein any R₄ and R₅ attached to the same nitrogen atom can together form a moiety of formula —(CH₂)_{q}— where q is 2, 3, 4, 5 or 6.

23. The process of claim 22, wherein each Q is CH.

24. The process of claim 22, wherein one Q is N.

25. The process of claim 20, wherein the coupling reagent is BOP.

26. The process of claim 20, wherein the coupling reagent is PyAOP.

27. A synthetic process comprising:
(a) reacting a compound of Formula Ht-OH with a coupling reagent of Formula: 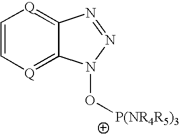
wherein:
one Q is CH, and one Q is CH or N;
each R₄ and R₅ is independently C₁₋₆ alkyl;
and wherein any R₄ and R₅ attached to the same nitrogen atom can together form a moiety of formula —(CH₂)_{q}— where q is 2, 3, 4, 5 or 6;
in the presence of a base, to form a compound of Formula II:
Ht-(O)ₖ-L II
or a salt thereof, wherein:
k is 0 or 1;
Ht is a heterocycle of Formula a, b, c or d: 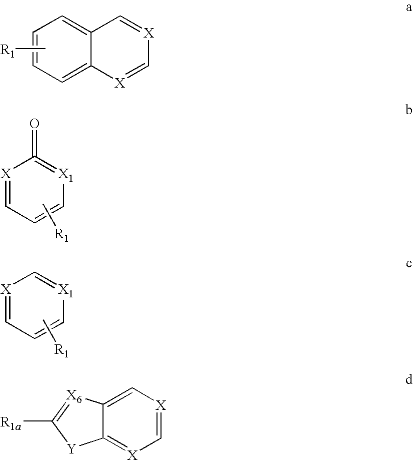
R₁, R₁ₐ and R_{1b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;
each X is independently N or CH;
X₆ is CR_{1b} or N;
X₁ is NH or CH₂;
Y is S or O;
L is a group having the Formula: 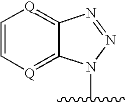
wherein one Q is CH, and one Q is CH or N; and
(b) reacting the compound of Formula II with a compound of Formula Ar—B(OH)₂;
wherein Ar has one of the Formulas e-j: 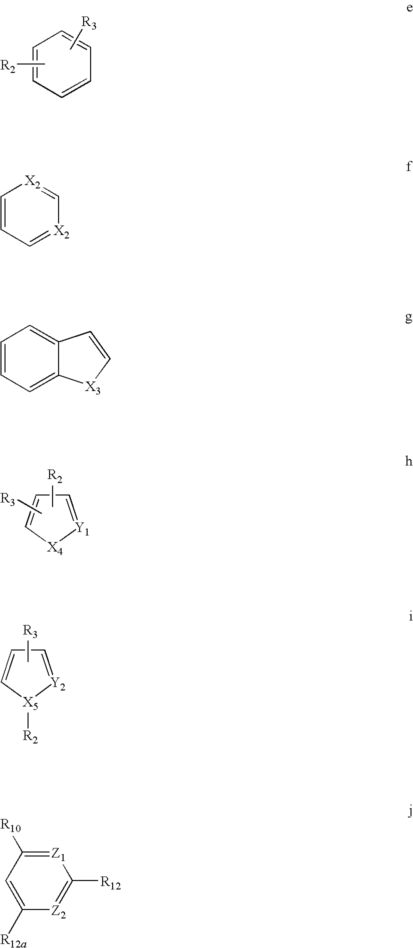
wherein:
each X₂ is independently N or CH;
X₃ is NH or CH₂;
X₄ is NH, S or O;
X₅ is N or CH;
Y₁ is N or CH;
Y₂ is N or CH;
R₂ and R₃ are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;
Z₁ is CR₁₁ₐ or N;
Z₂ is CR_{11b} or N; and
R₁₀, R₁₁ₐ, R_{11b}, R₁₂ and R₁₂ₐ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;
wherein the reaction is performed in the presence of a base;
and optionally in the presence of:
(i) oxygen; or
(ii) a Pd(0) catalyst; or
(iii) both oxygen and a Pd(0) catalyst;
for a time and under conditions effective to form a compound of Formula Ht-O—Ar.

28. The process of claim 27, wherein Ht has the Formula a, wherein each X is N.

29. The process of claim 27, wherein Ht has the Formula d, wherein each X is N and Y is S.

30. The process of claim 27, wherein Ht has the Formula c, wherein each X is N.

31. The process of claim 27, wherein Ht has the Formula b, wherein X is N, and X₁ is NH.

32. The process of claim 27, wherein Ar has the Formula e.

33. The process of claim 27, wherein Ar has the Formula f, wherein one X₂ is N and the other X₂ is CH.

34. The process of claim 27, wherein Ar has the Formula f, wherein each X₂ is N.

35. The process of claim 27, wherein Ar has the Formula g, wherein X₃ is NH.

36. The process of claim 27, wherein Ar has the Formula h, wherein X₄ is 0, and Y₁ is N.

37. The process of claim 27, wherein Ar has the Formula i.

38. The process of claim 27, wherein Ar has the Formula j.

39. The process of claim 27, wherein Ht has the Formula a wherein each X is N, and Ar has the Formula j.

40. A compound of Formula III: 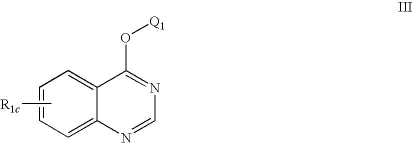
wherein:
R_{1c} is H, C₁₋₁₄ alkyl, C₁₋₁₄ alkoxy, halogen, C₇₋₂₀ arylalkyl, cyano, nitro, C₂₋₁₄ carboalkoxy, C₂₋₁₄ alkanoyl or trihaloalkyl;
Q₁ is selected from formulas j, k, m, n and o: 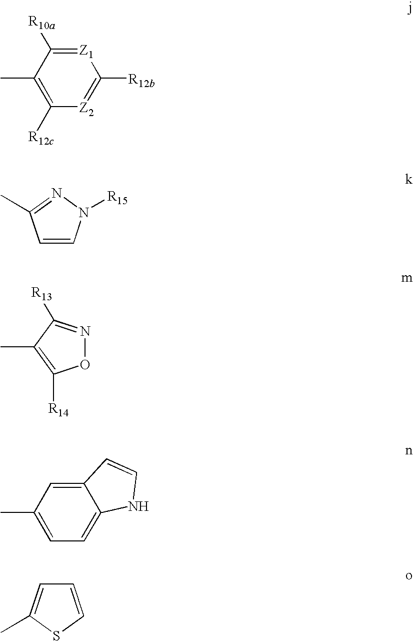
Z₁ is CR₁₁ₐ or N;
Z₂ is CR_{11b} or N;
R₁₀ₐ, R₁₁ₐ, R_{11b}, R_{12b} and R_{12c} are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;
R₁₃ and R₁₄ are each independently selected from the group consisting of H, C₁₋₁₄ alkyl and C₇₋₂₄ arylalkyl; and
R₁₅ is H, C₁₋₁₄ alkyl, C₇₋₂₄ arylalkyl, or C₁₋₆ trihaloalkyl;
provided that:
(i) when R₁₀ₐ and R_{12c} are each H, and Z₁ and Z₂ are each CH, then R_{12b} is not NO₂; and
(ii) when R_{12b} is H, and Z₁ and Z₂ are each CH, then neither R₁₀ₐ nor R_{12c} is NO₂.

41. The compound of claim 40, wherein Q₁ has the Formula j, wherein Z₁ is CR₁₁ₐ and Z₂ is CR_{11b}.

42. The compound of claim 41, wherein Z₁ and Z₂ are each CH; and R_{12b} is H.

43. The compound of claim 41, wherein Z₁ is CR₁₁ₐ, Z₂ is CR_{11b}, and R₁₀ₐ and R_{12b} are each H.

44. The compound of claim 41, wherein Z₁ and Z₂ are each CH; and R₁₀ₐ and R_{12c} are each H.

45. The compound of claim 41, wherein R_{12b} is selected from the group consisting of CF₃, —C(═O)CH₃, —C(═O)CH₂CH₃, —OCH₃, —OCH₂CH₃, —CH₃, —CH₂CH₃, CN, halogen and C₇₋₂₄ arylalkyl.

46. The compound of claim 44, wherein R_{12b} is selected from the group consisting of CF₃, —C(═O)CH₃, —C(═O)CH₂CH₃, —OCH₃, —OCH₂CH₃, —CH₃, —CH₂CH₃, CN, halogen and C₇₋₂₄ arylalkyl.

47. The compound of claim 45, wherein Z₁ and Z₂ are each CH.

48. The compound of claim 40, wherein Z₁ is CR₁₁ₐ, and Z₂ is N.

49. The compound of claim 48, wherein R_{12b} is halogen.

50. The compound of claim 48, wherein R_{12c} is alkoxy.

51. The compound of claim 40, wherein Q₁ has the Formula k.

52. The compound of claim 40, wherein Q₁ has the Formula m.

53. The compound of claim 40, wherein Q₁ has the Formula n.

54. The compound of claim 40, wherein Q₁ has the Formula o.

55. A compound of Formula IV: 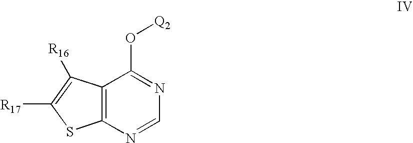
wherein:
R₁₆ and R₁₇ are each independently H, C₁₋₁₄ alkyl, carboxy, C₂₋₁₄ carboalkoxy, C(═O)CH₃, —C(═O)CH₂CH₃, C₂₋₁₄ alkanoyl or C₇₋₂₄ arylalkyl; and
Q₂ has the Formula: 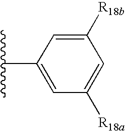
R₁₈ₐ and R_{18b} are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl, C₁₋₆ trihaloalkyl, N(R₅₀)(R₅₁), —NH₂, —NH(C₁-C₆ alkyl), —N(C₁-C₆ alkyl)(C₁-C₆ alkyl), —N(C₁-C₃ alkyl)C(O)(C₁-C₆ alkyl), —NHC(O)(C₁-C₆ alkyl), —NHC(O)H, —C(O)NH₂, —C(O)NH(C₁-C₆ alkyl), —C(O)N(C₁-C₆ alkyl)(C₁-C₆ alkyl), —CN, —OH, —C(O)OC₁-C₆ alkyl, —C(O)C₁-C₆ alkyl, C₆-C₁₄ aryl or C₄-C₁₀ heteroaryl;
R₅₀ and R₅₁ are each independently H, C₁₋₁₄ alkyl, C₂₋₁₄ alkenyl, C₂₋₁₄ alkynyl, C₇₋₂₄ arylalkyl, C₂₋₁₄ alkanoyl, C₁₋₆ trihaloalkyl, —S(O)ᵥ—C₁₋₁₄ alkyl; —S(O)ᵥ—C₆₋₁₄ aryl, —S(O)ᵥ—C₇₋₂₄ arylalkyl or —S(O)ᵥ—C₇₋₂₄ alkylaryl; where v is 0, 1 or 2;
or R₅₀ and R₅₁ together can form a moiety of formula —(CH₂)ᵣ— where r is 2, 3, 4, 5 or 6;
or Q₂ has the Formula m: 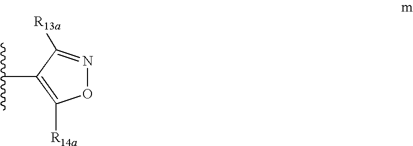
where R₁₃ₐ and R₁₄ₐ are each independently selected from the group consisting of H, C₁₋₁₄ alkyl and C₇₋₂₄ arylalkyl.

56. The compound of claim 55, wherein Q₂ has the Formula:

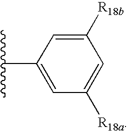

57. The compound of claim 56, wherein R₁₈ₐ and R_{18b} are each independently selected from H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl.

58. The compound of claim 55, wherein Q₂ has the Formula m:

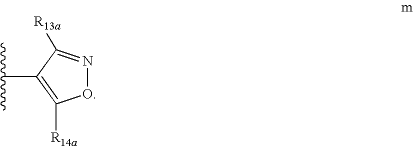

59. The compound of claim 58, wherein R₁₃ₐ and R₁₄ₐ are each C₁₋₁₄ alkyl.

60. A compound of Formula V: 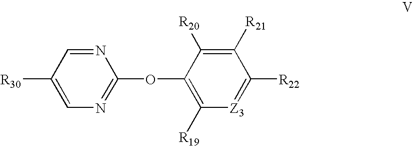
wherein:
Z₃ is N or CR_{11c};
R_{11c} is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₁₉ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₀ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₁ is H, F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl;
R₂₂ is H F, Cl, Br, I, C₁₋₁₄ alkyl, C₁₋₁₄ alkoxy, C₁₋₁₄ alkanoyl, C₂₋₁₄ alkenyl, C₂₋₁₄ carboalkoxy, or —S—C₁₋₁₄ alkyl; and
R₃₀ is halogen;
provided that:
(i) when Z₃ is CR_{11c}, then at least one of R₁₉, R₂₀, R₂₁ and R₂₂ is other than H; and
(ii) when Z₃ is N, the R_{11c}, R₁₉, R₂₀, R₂₁ and R₂₂ are each independently selected from H, C₂₋₁₄ alkenyl, —S—C₁₋₁₄ alkyl and C₁₋₁₄ alkoxy.

61. The compound of claim 60, wherein Z₃ is N.

62. The compound of claim 61, wherein R₃₀ is bromine.

63. The compound of claim 62, wherein R₁₉ is C₁₋₁₄ alkoxy.

64. The compound of claim 63, wherein R₂₀, R₂₁ and R₂₂ are each H.

65. The compound of claim 60, wherein Z₃ is CR_{11c}.

66. The compound of claim 65, wherein R_{11c}, R₁₉, R₂₀, R₂₁ and R₂₂ are each H.

67. A compound of Formula VI: 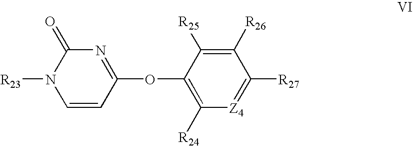
wherein:
(A) Z₄ is N;
R₂₃ is C₁₋₁₄ alkyl or C₇₋₂₄ arylalkyl; and
R₂₄, R₂₅, R₂₆ and R₂₇ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;
or
(B) Z₄ is CR_{11d};
R_{11d} is H or C₁₋₁₄ alkyl;
R₂₃ is C₁₋₁₄ alkyl; and
R₂₄, R₂₅, R₂₆ and R₂₇ are each independently selected from the group consisting of H, C₁₋₁₄ alkoxy, C₇₋₂₄ arylalkyl, cyano, C₂₋₁₄-carboalkoxy, C₂₋₁₄ alkanoyl and C₁₋₆ trihaloalkyl.

68. The compound of claim 67, wherein Z₄ is N.

69. The compound of claim 68, wherein R₂₄ is C₁₋₁₄ alkoxy.

70. The compound of claim 68, wherein R₂₄, R₂₅, R₂₆ and R₂₇ are each independently selected from H, C₁₋₁₄ alkoxy, cyano and C₁₋₆ trihaloalkyl.

71. The compound of claim 68, wherein R₂₄, R₂₅, R₂₆ and R₂₇ are each independently selected from H, OCH₃, cyano and CF₃.

72. The compound of claim 67, wherein Z₄ is CR_{11d}.

73. The compound of claim 72, wherein R₂₄ is C₁₋₁₄ alkoxy.

74. The compound of claim 73, wherein R₂₃ is methyl; and R₂₄ is methoxy.

75. A method for treating an oncological disease or disorder, comprising administering to a patient in need thereof a therapeutically effective amount of a compound of any of claims 40, 55, 60 or 67.

76. A method for treating inflammation, comprising administering to a patient in need thereof a therapeutically effective amount of a compound of any of claims 40, 55, 60 or 67.

77. The process of claim 27, wherein k is 1.

78. The process of claim 27, wherein k is 0.

79. The process of claim 27, wherein said base is a metal carbonate.

80. The process of claim 27, wherein said base is Cs₂CO₃.

81. The product of the process of claim 1.

82. The process of claim 27, wherein the reacting is performed in the presence of both oxygen and a palladium (0) catalyst.

83. The process of claim 1, wherein the base comprises a metal carbonate, metal bicarbonate, or a phosphate.

84. The process of claim 83, wherein the base comprises Cs₂CO₃.

85. The process of claim 1, wherein the reaction is performed in a solvent system comprising one or more organic solvents.

86. The process of claim 85, wherein the solvent system further comprises water in an amount of up to about 12% v/v.

87. A synthetic process comprising:
reacting a compound of Formula XX: 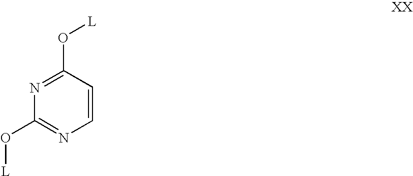
or a salt thereof, wherein:
L is a group having the Formula: 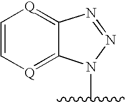
wherein one Q is CH, and one Q is CH or N;
with a compound of Formula Ar—B(OH)₂;
wherein Ar has one of the Formulas e-j: 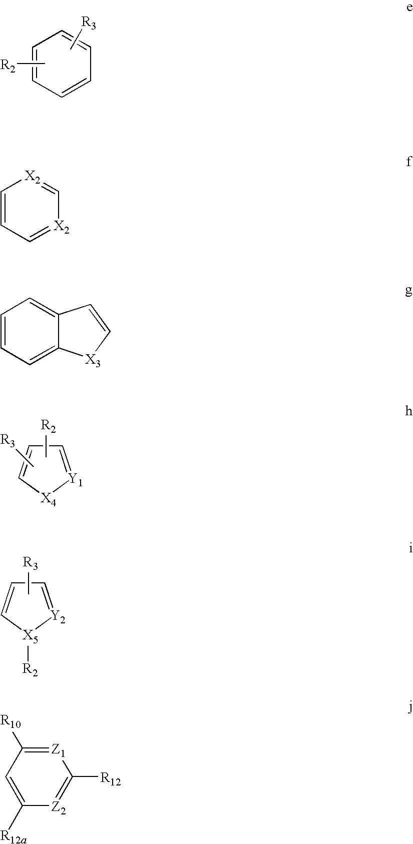
wherein:
each X₂ is independently N or CH;
X₃ is NH or CH₂;
X₄ is NH, S or O;
X₅ is N or CH;
Y₁ is N or CH;
Y₂ is N or CH;
R₂ and R₃ are each independently H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl or C₁₋₆ trihaloalkyl;
Z₁ is CR₁₁ₐ or N;
Z₂ is CR_{11b} or N; and
R₁₀, R₁₁ₐ, R_{11b}, R₁₂ and R₁₂ₐ are each independently selected from the group consisting of H, C₁₋₁₄ alkyl, C₁₋₁₄ alkoxy, halogen, C₇₋₂₄ arylalkyl, cyano, nitro, C₂₋₁₄ carboalkoxy, C₂₋₁₄ alkanoyl and C₁₋₆ trihaloalkyl;
wherein the reaction is performed in the presence of a base;
and optionally in the presence of water;
and optionally in the presence of one or more of:
(i) oxygen;
(ii) a Pd(0) catalyst; or
(iii) H₂O₂;
for a time and under conditions effective to form a compound of Formula XXI:

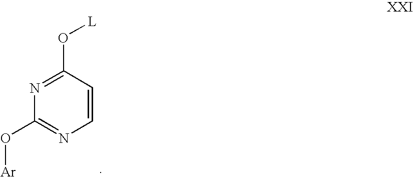

88. The process of claim 87, further comprising reacting the compound of Formula XXI with a compound of Formula Ar′—B(OH)₂ to provide a compound of Formula XXII: 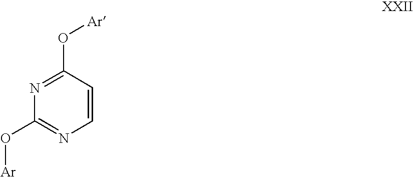
wherein Ar′ is an independently selected moiety of Formula Ar.

89. A compound of Formula XXX: 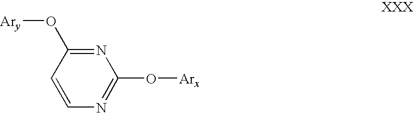
wherein:
Arₓ is phenyl or pyridyl, each of which is optionally substituted with up to 3 substituents selected from F, Cl, Br, I, C₁₋₁₄ alkyl, C₁₋₁₄ alkoxy, C₁₋₁₄ alkanoyl, NO₂, and C₂₋₁₄ carboalkoxy; and
Ar_{y} is phenyl or pyridyl, each of which is optionally substituted with up to 3 substituents selected from F, Cl, Br, I, C₁₋₁₄ alkyl, C₁₋₁₄ alkoxy, C₁₋₁₄ alkanoyl, NO₂, and C₂₋₁₄ carboalkoxy;
or Ar_{y} is: 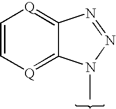
where one Q is CH, and one Q is CH or N.

90. A compound of formula XXXI: 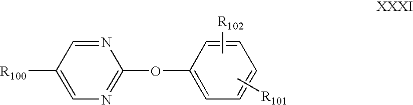
wherein:
R₁₀₀ is phenyl, optionally substituted with 1 or 2 substituents independently selected from F, Cl, Br, I, C₁₋₁₄ alkyl, C₁₋₁₄ alkoxy, C₁₋₁₄ alkanoyl, NO₂, and C₂₋₁₄ carboalkoxy; and
R₁₀₁ and R₁₀₂ are each independently selected from F, Cl, Br, I, C₁₋₁₄ alkyl, C₁₋₁₄ alkoxy, C₁₋₁₄ alkanoyl, NO₂, and C₂₋₁₄ carboalkoxy.
